(19) United States
(12) Patent Application Publication
(10) Pub. No.: US 2024/0299927 A1
(43) Pub. Date: Sep. 12, 2024

(54) FLUIDIC METHODS

(71) Applicant: Inflammatix, Inc., Sunnyvale, CA (US)

(72) Inventors: Edward K.Y. Jung, Bellevue, WA (US); Eric C. Leuthardt, St. Louis, MO (US); Royce A. Levien, Cambridge, MA (US); Robert W. Lord, Seattle, WA (US); Mark A. Malamud, Seattle, WA (US); John D. Rinaldo, JR., Bellevue, WA (US); Lowell L. Wood, JR., Bellevue, WA (US)

(73) Assignee: Inflammatix, Inc., Sunnyvale, CA (US)

(21) Appl. No.: 18/664,790

(22) Filed: May 15, 2024

Related U.S. Application Data
(63) Continuation of application No. 17/005,065, filed on Aug. 27, 2020, now abandoned.
Application No. 17/005,065 is a continuation of application No. 11/906,168, filed on Sep. 28, 2007, now abandoned.
Application No. 11/906,168 is a continuation-in-part of application No. 11/906,171, filed on Sep. 28, 2007, now abandoned.
Application No. 11/906,171 is a continuation-in-part of application No. 11/900,660, filed on Sep. 11, 2007, now Pat. No. 8,068,991.
Application No. 11/900,660 is a continuation-in-part of application No. 11/900,649, filed on Sep. 11, 2007, now abandoned.
Application No. 11/900,649 is a continuation-in-part of application No. 11/900,637, filed on Sep. 11, 2007, now abandoned.
Application No. 11/900,637 is a continuation-in-part of application No. 11/799,462, filed on Apr. 30, 2007, now abandoned.
Application No. 11/799,462 is a continuation-in-part of application No. 11/729,301, filed on Mar. 27, 2007, now abandoned.
Application No. 11/729,301 is a continuation-in-part of application No. 11/729,276, filed on Mar. 27, 2007, now abandoned.

(51) Int. Cl. B01L 3/02; A61B 5/145; G01N 1/40

(52) U.S. Cl. CPC B01L 3/0268; A61B 5/145; G01N 1/4077; B01L 2400/0439; B01L 2400/0487; B01L 2400/0605; G01N 2001/4072

(57) ABSTRACT

Techniques for treating a detrimental physical condition are disclosed. In one embodiment, a method includes processing one or more samples obtained from the individual through use of one or more microfluidic chips to provide data indicating one or more constituents present within the one or more samples, and processing the data to determine a quantity of at least one constituent that indicates the detrimental physical condition of the individual. Next, the method determines at least one dosage of at least one treatment agent for mitigation of the detrimental physical condition based on the quantity of the at least one constituent that indicates the detrimental physical condition, and facilitates an administration of the at least one dosage for mitigation of the detrimental physical condition.

### CROSS-REFERENCE TO RELATED APPLICATIONS

This application is a continuation of U.S. patent application Ser. No. 17/005,065, filed 27 Aug. 2020, which is a continuation of U.S. patent application Ser. No. 11/906,168, filed 28 Sep. 2007, which is a continuation-in-part of U.S. patent application Ser. No. 11/906,171, filed 28 Sep. 2007, which is a continuation-in-part of U.S. patent application Ser. No. 11/900,660, filed 11-Sep.-2007, now issued as U.S. Pat. No. 8,068,991, which is a continuation-in-part of Ser. No. 11/900,649, filed 11 Sep. 2007, which is a continuation-in-part of U.S. patent application Ser. No. 11/900,637, filed 11 Sep. 2007, which is a continuation-in-part of U.S. patent application Ser. No. 11/799,462, filed 30 Apr. 2007, which is a continuation-in-part of U.S. patent application Ser. No. 11/729,301, filed 27-Mar.-2007, which is a continuation-in-part of U.S. patent application Ser. No. 11/729,276, filed 27 Mar. 2007, each of which is incorporated in its entirety by this reference.

### TECHNICAL FIELD

The present disclosure relates to methods and devices that may be used to separate components from one or more samples.

### SUMMARY

In some embodiments one or more methods are provided that include placing one or more sample fluids into one or more separation channels so that the one or more sample fluids are in substantially laminar flow with one or more magnetically active fluids and translocating one or more magnetically active constituents from the one or more sample fluids into the one or more magnetically active fluids. The method may optionally include mixing one or more magnetically active agents with the one or more sample fluids to form the one or more magnetically active constituents. The method may optionally include detecting one or more constituents of the one or more sample fluids. In addition to the foregoing, other aspects are described in the claims, drawings, and text forming a part of the present disclosure.

In some embodiments one or more methods are provided that include placing one or more sample fluids into one or more separation channels so that the one or more sample fluids are in substantially laminar flow with one or more separation fluids and translocating one or more magnetically active constituents from the one or more sample fluids into the one or more separation fluids through use of one or more magnets. The method may optionally include mixing one or more magnetically active agents with the one or more sample fluids to form the one or more magnetically active constituents. The method may optionally include detecting one or more constituents of the one or more sample fluids. In addition to the foregoing, other aspects are described in the claims, drawings, and text forming a part of the present disclosure.

In some embodiments one or more methods are provided that include placing one or more sample fluids into one or more separation channels so that the one or more sample fluids are in substantially laminar flow with one or more first separation fluids and one or more second separation fluids, translocating one or more magnetically active constituents from the one or more sample fluids into the one or more first separation fluids, and translocating the one or more magnetically active constituents from the one or more sample fluids into the one or more second separation fluids. In addition to the foregoing, other aspects are described in the claims, drawings, and text forming a part of the present disclosure.

In some embodiments one or more devices are provided that include one or more first inlets, one or more second inlets, one or more outlets, one or more magnetically active fluids, and one or more separation channels that are configured to facilitate substantially laminar adjacent flow of one or more first fluids and the one or more magnetically active fluids within the one or more separation channels. The devices may optionally include one or more magnets. In addition to the foregoing, other aspects are described in the claims, drawings, and text forming a part of the present disclosure.

In some embodiments one or more devices are provided that include one or more inlets, one or more outlets, one or more substantially continuous fluid channels, one or more magnetically active fluids, and one or more separation channels that are configured to facilitate substantially laminar adjacent flow of one or more first fluids and the one or more magnetically active fluids within the one or more separation channels. In addition to the foregoing, other aspects are described in the claims, drawings, and text forming a part of the present disclosure.

In some embodiments one or more devices are provided that include one or more first inlets, one or more second inlets, one or more outlets, one or more magnets, and one or more separation channels that are configured to facilitate substantially laminar adjacent flow of one or more first fluids and one or more second fluids within the one or more separation channels. In addition to the foregoing, other aspects are described in the claims, drawings, and text forming a part of the present disclosure.

In some embodiments one or more devices are provided that include one or more inlets, one or more outlets, one or more substantially continuous fluid channels, one or more separation channels that are configured to facilitate substantially laminar adjacent flow of one or more first fluids and one or more second fluids within the one or more separation channels, and one or more magnets. In addition to the foregoing, other aspects are described in the claims, drawings, and text forming a part of the present disclosure.

In some embodiments, means include but are not limited to circuitry and/or programming for effecting the herein referenced functional aspects; the circuitry and/or programming can be virtually any combination of hardware, software, and/or firmware configured to effect the herein referenced functional aspects depending upon the design choices of the system designer. In addition to the foregoing, other system aspects means are described in the claims, drawings, and/or text forming a part of the present disclosure.

In some embodiments, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer. In addition to the foregoing, other system aspects are described in the claims, drawings, and/or text forming a part of the present application.

The foregoing summary is illustrative only and is not intended to be in any way limiting. In addition to the illustrative aspects, embodiments, and features described above, further aspects, embodiments, and features will become apparent by reference to the drawings, claims, and the following detailed description.

### BRIEF DESCRIPTION OF THE FIGURES

FIG. 1 illustrates an example system 100 in which embodiments may be implemented.

FIG. 2 illustrates an operational flow representing example operations related to methods for separating one or more constituents from one or more samples.

FIG. 3 illustrates alternate embodiments of the example operational flow of FIG. 2.

FIG. 4 illustrates alternate embodiments of the example operational flow of FIG. 2.

FIG. 5 illustrates alternate embodiments of the example operational flow of FIG. 2.

FIG. 6 illustrates alternate embodiments of the example operational flow of FIG. 2.

FIG. 7 illustrates alternate embodiments of the example operational flow of FIG. 2.

FIG. 8 illustrates alternate embodiments of the example operational flow of FIG. 2.

FIG. 9 illustrates an operational flow representing example operations related to methods for separating one or more constituents from one or more samples.

FIG. 10 illustrates alternate embodiments of the example operational flow of FIG. 9.

FIG. 11 illustrates alternate embodiments of the example operational flow of FIG. 9.

FIG. 12 illustrates alternate embodiments of the example operational flow of FIG. 9.

FIG. 13 illustrates alternate embodiments of the example operational flow of FIG. 9.

FIG. 14 illustrates alternate embodiments of the example operational flow of FIG. 9.

FIG. 15 illustrates alternate embodiments of the example operational flow of FIG. 9.

FIG. 16 illustrates an operational flow representing example operations related to methods for separating one or more constituents from one or more samples.

FIG. 17 illustrates alternate embodiments of the example operational flow of FIG. 16.

FIG. 18 illustrates alternate embodiments of the example operational flow of FIG. 16.

FIG. 19 illustrates alternate embodiments of the example operational flow of FIG. 16.

FIG. 20 illustrates alternate embodiments of the example operational flow of FIG. 16.

FIG. 21 illustrates an example system 2100 in which embodiments may be implemented.

FIG. 22 illustrates alternate embodiments of the system of FIG. 21.

FIG. 23 illustrates alternate embodiments of the system of FIG. 21.

FIG. 24 illustrates alternate embodiments of the system of FIG. 21.

FIG. 25 illustrates alternate embodiments of the system of FIG. 21.

FIG. 26 illustrates alternate embodiments of the system of FIG. 21.

FIG. 27 illustrates alternate embodiments of the system of FIG. 21.

FIG. 28 illustrates an example system 2800 in which embodiments may be implemented.

FIG. 29 illustrates alternate embodiments of the system of FIG. 28.

FIG. 30 illustrates alternate embodiments of the system of FIG. 28.

FIG. 31 illustrates alternate embodiments of the system of FIG. 28.

FIG. 32 illustrates alternate embodiments of the system of FIG. 28.

FIG. 33 illustrates alternate embodiments of the system of FIG. 28.

FIG. 34 illustrates an example system 3400 in which embodiments may be implemented.

FIG. 35 illustrates alternate embodiments of the system of FIG. 34.

FIG. 36 illustrates alternate embodiments of the system of FIG. 34.

FIG. 37 illustrates alternate embodiments of the system of FIG. 34.

FIG. 38 illustrates alternate embodiments of the system of FIG. 34.

FIG. 39 illustrates alternate embodiments of the system of FIG. 34.

FIG. 40 illustrates an example device 4000 in which embodiments may be implemented.

FIG. 41 illustrates alternate embodiments of the device of FIG. 40.

FIG. 42 illustrates alternate embodiments of the device of FIG. 40.

FIG. 43 illustrates alternate embodiments of the device of FIG. 40.

FIG. 44 illustrates alternate embodiments of the device of FIG. 40.

FIG. 45 illustrates alternate embodiments of the device of FIG. 40.

FIG. 46A illustrates an example separation channel 4600.

FIG. 46B illustrates an example separation channel 4650.

FIG. 47A illustrates an example separation channel 4700.

FIG. 47B illustrates an example separation channel 4750.

FIG. 48A illustrates an example separation channel 4800.

FIG. 48B illustrates an example separation channel 4850.

FIG. 49A illustrates an example separation channel 4900.

FIG. 49B illustrates an example separation channel 4950.

FIG. 50A illustrates an example separation channel 5000.

FIG. 50B illustrates an example separation channel 5050.

FIG. 51 illustrates example separation channels 5100.

FIG. 52 illustrates example separation channels 5200.

FIG. 53 illustrates example separation channels 5300.

FIG. 54 illustrates example separation channels 5400.

FIG. 55 illustrates an example system 5500.

FIG. 56 illustrates an example system 5600.

FIG. 57 illustrates an example system 5700.

FIG. 58 illustrates an example system 5800.

FIG. 59 illustrates an example system 5900.

FIG. 60 illustrates an example system 6000.

FIG. 61 illustrates an example system 6100.

FIG. 62 illustrates an example system 6200.

FIG. 63 illustrates an example system 6300.

FIG. 64 illustrates an example system 6400.

FIG. 65 illustrates an example system 6500.

FIG. 66 illustrates an example system 6600.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

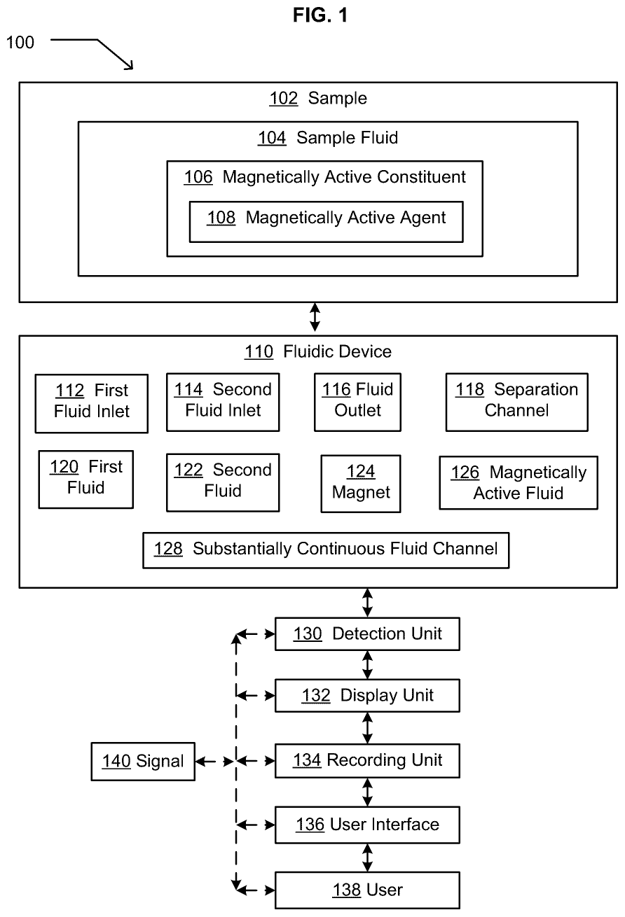

FIG. 1 illustrates an example system 100 in which embodiments may be implemented. In some embodiments, one or more constituents within one or more samples 102 may be separated. In some embodiments, one or more constituents within one sample 102 may be separated. In some embodiments, one or more fluidic devices 110 may be used to separate one or more constituents within one or more samples 102. In some embodiments, one or more fluidic devices 110 may be configured to operably associate with one or more detection units 130. In some embodiments, one or more fluidic devices 110 may be configured to operably associate with one or more display units 132. In some embodiments, one or more detection units 130 may be portable detection units 130. In some embodiments, one or more detection units 130 may be non-portable detection units 130. In some embodiments, one or more detection units 130 may be hand-held detection units 130. In some embodiments, one or more detection units 130 may include one or more user interfaces 136. In some embodiments, one or more detection units 130 may include one user interface 136. In some embodiments, one or more detection units 130 may include one or more user interfaces 136 that are directly coupled with the one or more detection units 130. In some embodiments, one or more detection units 130 may include one or more user interfaces 136 that are remotely coupled with one or more detection units 130. For example, in some embodiments, a user 138 may interact with the one or more detection units 130 through direct physical interaction with the one or more detection units 130. In other embodiments, a user 138 may interact with one or more detection units 130 through remote interaction. In some embodiments, one or more detection units 130 may include one or more display units 132. In some embodiments, one or more detection units 130 may be directly coupled to one or more display units 132. In some embodiments, one or more detection units 130 may be remotely coupled to one or more display units 132. In some embodiments, one or more display units 132 may include one or more user interfaces 136. In some embodiments, one or more display units 132 may include one user interface 136.

### Sample

System 100 may be used in association with numerous types of samples 102. In some embodiments, one or more samples 102 may include a liquid. In some embodiments, one or more samples 102 may include a solid. In some embodiments, one or more solids may be suspended in one or more fluids to form one or more sample 102 fluids. In some embodiments, one or more samples 102 may include a semi-solid. Examples of such samples 102 include, but are not limited to, water, food, food products, solids, biological samples 102, samples 102 obtained from humans, environmental samples 102, or substantially any combination thereof. In some embodiments, one or more samples 102 may be associated with an individual. For example, in some embodiments, system 100 may be used for diagnostic purposes. In some embodiments, one or more samples 102 may be mixed with one or more magnetically active agents 108 that associate (e.g., bind) with one or more constituents that may be present within one or more samples 102 to form one or more magnetically active constituents 106.

### Fluidic Device

Fluidic devices 110 may be configured in numerous ways. For example, in some embodiments, a fluidic device may be configured as a microfluidic device. Methods used to construct microfluidic chips may be adapted to construct fluidic devices 110. Such methods have been described (e.g., U.S. Statutory Invention Registration No. H201; U.S. Pat. Nos. 6,454,945; 6,818,435; 6,812,458; 6,794,196; 6,709,869; 6,582,987; 6,482,306; 5,726,404; 7,118,910; 7,081,192; herein incorporated by reference).

In some embodiments, a fluidic device may be configured to utilize microfluidic principles. Accordingly, in some embodiments, a fluidic device may be configured to include one or more channels with at least one dimension that is less than 1 millimeter. However, in some embodiments, fluidic devices 110 may be configured such that they do not utilize microfluidic principles. Accordingly, in some embodiments, fluidic devices 110 may be configured such that there are not any components that have a dimension that is less than 1 millimeter. Accordingly, in some embodiments, fluidic devices 110 may be configured that include components having a dimension that is less than 1 millimeter, while in other embodiments, fluidic devices 110 may be configured with components having dimensions that are greater than 1 millimeter. In some embodiments, a fluidic device may include at least one component that has at least one dimension that is less than 1 millimeter and at least one component having at least one dimension that is greater than 1 millimeter. In some embodiments, fluidic devices 110 may be used in association with one or more pumps. In some embodiments, fluidic devices 110 may utilize capillary action to facilitate movement of fluids.

Fluidic devices 110 may be used in association with numerous methods. For example, in some embodiments, one or more fluidic devices 110 may be used in association with: chemiluminescent methods (e.g., U.S. Pat. Nos. 6,090,545 and 5,093,268; herein incorporated by reference), plasmon resonance sensors (e.g., U.S. Pat. No. 7,030,989; herein incorporated by reference), nuclear magnetic resonance detectors (e.g., U.S. Pat. No. 6,194,900; herein incorporated by reference), gradient-based assays (e.g., U.S. Pat. No. 7,112,444; herein incorporated by reference), reporter beads (e.g., U.S. Pat. No. 5,747,349; herein incorporated by reference), transverse electrophoresis (e.g., Macounova et al., Analytical Chemistry, 73:1627-1633 (2001)); isoelectric focusing (e.g., Macounova et al., Analytical Chemistry, 72:3745-3751 (2000); Xu et al., Isoelectric focusing of green fluorescent proteins in plastic microfluidic channels. Abstracts of Papers of the American Chemical Society, 219:9-ANYL (2000); Macounova et al., Analytical Chemistry, 73:1627-1633 (2001)), diffusion based systems (e.g., Kamholz et al., Biophysical Journal, 80:1967-1972 (2001); Hatch et al., Nature Biotechnology, 19:461-465 (2001); U.S. Pat. Nos. 6,221,677; 5,972,710; herein incorporated by reference), high performance liquid chromatography (e.g., U.S. Pat. No. 6,923,907; herein incorporated by reference), polynucleotide analysis (e.g., Belgrader et al., Biosensors & Bioelectronics, 14:849-852 (2000); Buchholz et al., Analytical Chemistry, 73:157-164 (2001); Fan et al., Analytical Chemistry, 71:4851-4859 (1999); Koutny et al., Analytical Chemistry, 72:3388-3391 (2000); Lee et al., Microfabricated plastic chips by hot embossing methods and their applications for DNA separation and detection. Sensors and Actuators B-Chemical, 75:142-148 (2001); U.S. Pat. No. 6,958,216; herein incorporated by reference), capillary electrophoresis (e.g., Kameoka et al., Analytical Chemistry, 73:1935-1941 (2001)), immunoassays (e.g., Hatch et al., Nature Biotechnology, 19:461-465 (2001); Eteshola and Leckband, D. Development and characterization of an ELISA assay in PDMS microfluidic channels. Sensors and Actuators B-Chemical 72:129-133 (2001); Cheng et al., Analytical Chemistry, 73:1472-1479 (2001); Yang et al., Analytical Chemistry, 73:165-169 (2001)), flow cytometry (e.g., Sohn et al., Proc. Natl. Acad. Sci., 97:10687-10690 (2000)), PCR amplification (e.g., Belgrader et al., Biosensors & Bioelectronics, 14:849-852 (2000); Khandurina et al., Analytical Chemistry, 72:2995-3000 (2000); Lagally et al., Analytical Chemistry, 73:565-570 (2001)), cell manipulation (e.g., Glasgow et al., IEEE Transactions On Biomedical Engineering, 48:570-578 (2001)), cell separation (e.g., Yang et al., Analytical Chemistry, 71:911-918 (1999)), cell patterning (e.g., Chiu et al., Proc. Natl. Acad. Sci., 97:2408-2413 (2000); Folch et al., Journal of Biomedical Materials Research, 52:346-353 (2000)), chemical gradient formation (e.g., Dertinger et al., Analytical Chemistry, 73:1240-1246 (2001); Jeon et al., Langmuir, 16:8311-8316 (2000)), microcantilevers (e.g., U.S. Pat. Nos. 7,141,385; 6,935,165; 6,926,864; 6,763,705; 6,523,392; 6,325,904; herein incorporated by reference), or substantially any combination thereof.

In some embodiments, one or more fluidic devices 110 may be configured to utilize one or more magnets. For example, in some embodiments, ferrous particles may be associated with one or more constituents that are associated with one or more samples 102 (e.g., use of antibodies, aptamers, polypeptides, polynucleotides, and the like that bind to the one or more constituents and that are coupled to a ferrous metallic particle). The one or more constituents may be separated from the remainder of the one or more samples 102 through use of one or more magnets. In some embodiments, one or more magnets 124 may be used to create eddy currents that may be used to separate one or more constituents from one or more samples 102. For example, in some embodiments, non-ferrous metallic particles may be associated with one or more constituents that are associated with one or more samples 102 (e.g., use of antibodies, aptamers, peptides, polynucleotides, and the like that bind to the one or more constituents and that are coupled to a non-ferrous metallic particle). One or more fluidic devices 110 may be configured such that passage of a non-ferrous metallic particle through a magnetic field will cause an eddy current to impart kinetic energy to the non-ferrous metallic particle and provide for separation of the associated constituents from the remainder of the one or more samples 102. In some embodiments, such methods may be combined with additional methods to provide for separation of one or more constituents from one or more samples 102. For example, magnetic separation may be used in combination with one or more additional methods that may include, but are not limited to, diffusion, filtration, precipitation, immunoassay, immunodiffusion, and the like. Examples of magnets 124 that may be used include, but are not limited to, electromagnets, permanent magnets, and substantially any combination thereof. Magnets 124 may be used in conjunction with numerous materials. Examples of such materials include, but are not limited to, ferromagnetic materials, diamagnetic materials, paramagnetic materials, and substantially any combination thereof.

In some embodiments, one or more fluidic devices 110 may be configured to utilize ferrofluids to separate one or more constituents from one or more samples 102. For example, in some embodiments, a fluidic device may include a separation channel 118 where a sample fluid 104 and a ferrofluid flow substantially in parallel (e.g., the sample fluid 104 and the ferrofluid flow side-by-side through the separation channel 118 (horizontal) and/or above and below (vertical)). In some embodiments, one or more fluidic devices 110 may include a ferrofluid having magnetic particles such that ferrous materials contained within the sample fluid 104 are attracted to the ferrofluid and thereby separated from the sample fluid 104. Accordingly, such fluidic devices 110 may be configured to separate one or more constituents from one or more samples 102. In some embodiments, one or more fluidic devices 110 may include a ferrofluid having ferrous particles such that magnetic materials contained within the sample fluid 104 are attracted to the ferrofluid and thereby separated from the sample fluid 104. Accordingly, in such embodiments, one or more fluidic devices 110 may be configured to utilize ferrofluids to separate one or more constituents from one or more samples 102.

### Detection Unit

Numerous types of detection units 130 may be used within system 100. Accordingly, numerous types of detection methods may be used within system 100. Examples of such detection methods include, but are not limited to, colorimetric methods, spectroscopic methods, resonance based methods, electron transfer based methods (redox), conductivity based methods, gravimetric based assays, turbidity based methods, ion-specific based methods, refractive index based methods, radiological based methods, or substantially any combination thereof. In some embodiments, a detection unit 130 may be stationary. For example, in some embodiments, a detection unit 130 may be a laboratory instrument. In some embodiments, a detection unit 130 may be portable. For example, in some embodiments, a detection unit 130 may be hand-held device.

### Display Unit

The system 100 may include one or more display units 132. Numerous types of display units 132 may be used in association with system 100. Examples of such display units 132 include, but are not limited to, liquid crystal displays, printers, audible displays, cathode ray displays, plasma display panels, Braille displays, passive displays, chemical displays, active displays, and the like. In some embodiments, display units 132 may display information in numerous languages. Examples of such languages include, but are not limited to, English, Spanish, German, Japanese, Chinese, Italian, and the like. In some embodiments, display units 132 may display information pictographically, colorometrically, and/or physically, such as displaying information in Braille. In some embodiments, one or more display units 132 may be physically coupled to one or more detection units 130. In some embodiments, one or more display units 132 may be remotely coupled to one or more detection units 130.

### Recording Unit

The system 100 may include one or more recording units 134. In some embodiments, one or more recording units 134 can communicate with one or more detection units 130, one or more display units 132, one or more user interfaces 136, and/or substantially any combination thereof. Many types of recording units 134 may be used within system 100. Examples of such recording devices include those that utilize a recordable medium that includes, but is not limited to, many types of memory, optical disks, magnetic disks, magnetic tape, and the like.

In some embodiments, one or more recording units 134 may be physically coupled to one or more detection units 130. In some embodiments, one or more recording units 134 may be physically coupled to one or more display units 132. In some embodiments, one or more recording units 134 may be remotely coupled to one or more detection units 130 and/or one or more display units 132. For example, in some embodiments, one or more recording units 134 may receive one or more signals 140 from one or more detection units 130 and/or one or more display units 132 that are remotely positioned relative to the one or more recording units 134. Accordingly, one or more recording units 134 may be positioned in one or more locations that are remote from the position where one or more fluidic devices 110, detection units 130, display units 132, or substantially any combination thereof are located.

### Signal

The system 100 may include one or more signals 140. Numerous types of signals 140 may be transmitted. Examples of such signals 140 include, but are not limited to, hardwired signals 140, wireless signals 140, infrared signals 140, optical signals 140, radiofrequency (RF) signals 140, audible signals 140, digital signals 140, analog signals 140, or substantially any combination thereof.

### User Interface/User

Numerous types of users 138 may interact with system 100. In some embodiments, a user 138 may be human. In some embodiments, a user 138 may be non-human. In some embodiments, a user 138 may interact with one or more systems 100 that include one or more fluidic devices 110, one or more detection units 130, one or more display units 132, one or more user interfaces 136, or substantially any combination thereof. The user 138 can interact through use of numerous types of user interfaces 136. For example, one or more users 138 may interact through use of numerous user interfaces 136 that utilize hardwired methods, such as through use of a keyboard, use of wireless methods, use of the internet, and the like. In some embodiments, a user 138 may be a health-care worker. Examples of such health-care workers include, but are not limited to, physicians, nurses, pharmacists, and the like. In some embodiments, a user 138 may be a hiker, a farmer, a food inspector, a cook, a traveler, and the like.

I. Methods for Separating One or More Constituents from One or More Samples

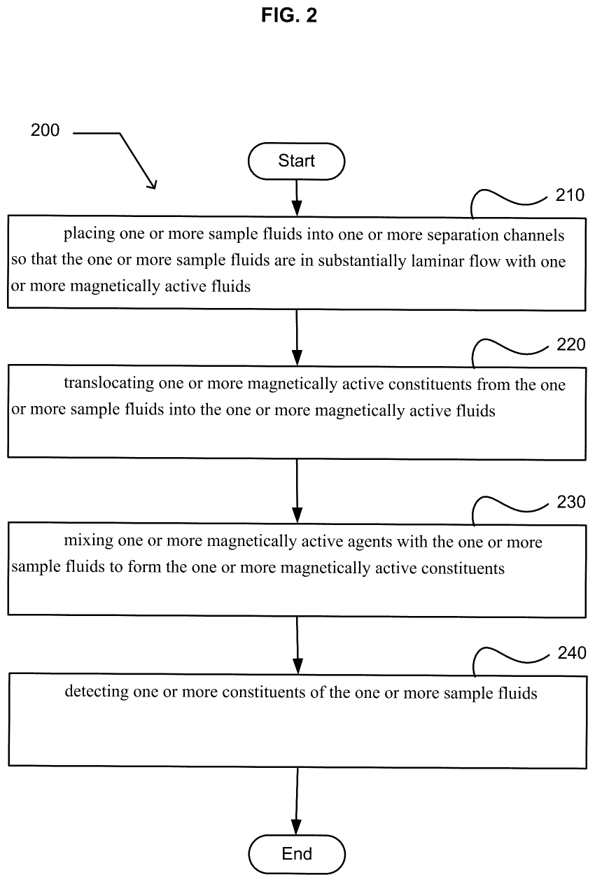

FIG. 2 illustrates an operational flow 200 representing examples of operations that are related to the performance of a method that may be used to separate one or more constituents from one or more samples 102. In FIG. 2 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 200 includes a placing operation 210 involving placing one or more sample fluids into one or more separation channels so that the one or more sample fluids are in substantially laminar flow with one or more magnetically active fluids. In some embodiments, placing operation 210 may include suspending the one or more samples in one or more fluids to form the one or more sample fluids. In some embodiments, placing operation 210 may include placing the one or more sample fluids that include one or more bodily samples into the one or more separation channels. In some embodiments, placing operation 210 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, placing operation 210 may include placing the one or more sample fluids that include blood into the one or more separation channels. In some embodiments, placing operation 210 may include placing the one or more sample fluids that include one or more environmental samples into the one or more separation channels. In some embodiments, placing operation 210 may include placing the one or more sample fluids that include one or more food samples into the one or more separation channels. In some embodiments, placing operation 210 may include placing the one or more sample fluids into the one or more separation channels so that the one or more sample fluids are in substantially parallel laminar flow with the one or more magnetically active fluids. In some embodiments, placing operation 210 may include placing the one or more sample fluids into the one or more separation channels so that the one or more sample fluids are in substantially anti-parallel laminar flow with the one or more magnetically active fluids.

After a start operation, the operational flow 200 includes a translocating operation 220 involving translocating one or more magnetically active constituents from the one or more sample fluids into the one or more magnetically active fluids. In some embodiments, translocating operation 220 may include translocating the one or more magnetically active constituents that include one or more non-ferrous tags. In some embodiments, translocating operation 220 may include translocating the one or more magnetically active constituents that include one or more ferrous tags. In some embodiments, translocating operation 220 may include translocating the one or more magnetically active constituents that include one or more magnetic tags. In some embodiments, translocating operation 220 may include translocating the one or more magnetically active constituents that include one or more paramagnetic tags. In some embodiments, translocating operation 220 may include translocating the one or more magnetically active constituents through use of one or more ferrofluids. In some embodiments, translocating operation 220 may include translocating the one or more magnetically active constituents through use of the one or more magnetically active fluids that include magnetic particles.

After a start operation, the operational flow 200 may optionally include a mixing operation 230 involving mixing one or more magnetically active agents with the one or more sample fluids to form the one or more magnetically active constituents. In some embodiments, mixing operation 230 may include mixing one or more magnetically active antibodies, aptamers, nucleic acids, ligands, or polypeptides, with the one or more sample fluids.

After a start operation, the operational flow 200 may optionally include a detecting operation 240 involving detecting one or more constituents of the one or more sample fluids. In some embodiments, detecting operation 240 may include detecting the one or more constituents with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, or immunoassay.

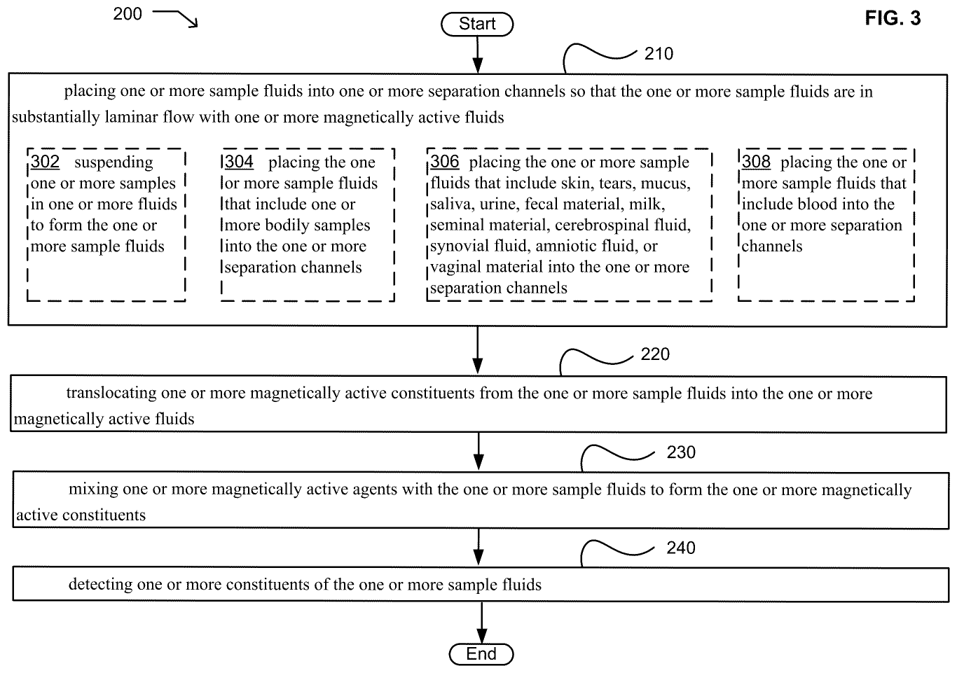

FIG. 3 illustrates alternative embodiments of the example operational flow 200 of FIG. 2. FIG. 3 illustrates example embodiments where the placing operation 210 may include at least one additional operation. Additional operations may include an operation 302, an operation 304, an operation 306, and/or an operation 308.

At operation 302, the placing operation 210 may include suspending the one or more samples in one or more fluids to form the one or more sample fluids. In some embodiments, one or more samples 102 may be suspended in one or more fluids to form one or more sample fluids 104. In some embodiments, one or more samples 102 may be dissolved in one or more solvents. Numerous types of samples 102 may be suspended in one or more fluids to form one or more sample fluids 104. Examples of samples 102 include, but are not limited to, solid samples 102, liquid samples 102, semi-solid samples 102, gels, and the like. Such samples 102 may include, but are not limited to, food samples 102, biological samples 102, fuel samples 102, environmental samples 102, crop samples 102, water samples 102, diagnostic samples 102 (e.g., tissue, blood, saliva, mucus, cerebrospinal fluid, amniotic fluid, and the like). In some embodiments, blood samples 102 may be combined with one or more fluids to form one or more sample fluids 104. In some embodiments, one or more fluids may include desired functions. For example, in some embodiments, one or more fluids that include nuclease inhibitors may be mixed with one or more samples 102. In some embodiments, one or more fluids that include one or more RNase inhibitors may be mixed with one or more blood samples 102 to preserve polyribonucleic acids present within the one or more blood samples 102. In some embodiments, precipitates may be suspended in one or more fluids. For example, in some embodiments, precipitated polynucleotides may be suspended in one or more fluids to form one or more sample fluids 104. In some embodiments, one or more fluids may be used to extract one or more components from a first sample. In some embodiments, one or more fluids may be selected to exhibit desired fluid characteristics. Examples of such characteristics include, but are not limited to, viscosity, density, miscibility, solubility, polarity, vapor pressure, flammability, and the like. Accordingly, numerous types of samples 102 and numerous types of fluids may be used to prepare a sample fluid 104.

At operation 304, the placing operation 210 may include placing the one or more sample fluids that include one or more bodily samples into the one or more separation channels. In some embodiments, one or more sample fluids 104 that include one or more bodily samples 102 may be placed into one or more separation channels 118. Examples of such bodily samples 102 include, but are not limited to, tissue, tears, saliva, mucus, wax, blood, synovial fluid, cerebrospinal fluid, seminal fluid, vaginal fluid, amniotic fluid, urine, fecal material, and the like. In some embodiments, such samples 102 may be used within diagnostic methods. For example, in some embodiments, amniotic fluid may be used to determine if a fetus exhibits a genotype associated with a disease. In other examples, parasites within fecal material may be detected to determine if an individual is infected with a parasite. Numerous pathogens and parasites have been described (e.g., U.S. patent application Ser. Nos. 11/729,301, 11/729,274, 11/729,276, herein incorporated by reference). Accordingly, numerous types of bodily samples 102 may be selected.

At operation 306, the placing operation 210 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, one or more sample fluids 104 that include skin, tears, mucus, saliva, urine, fecal material, milk, seminal material, cerebrospinal fluid, synovial fluid, amniotic fluid, vaginal material, or the like may be placed into one or more separation channels 118 in substantially any combination.

At operation 308, the placing operation 210 may include placing the one or more sample fluids that include blood into the one or more separation channels. In some embodiments, one or more sample fluids 104 that include blood may be placed into one or more separation channels 118. In some embodiments, one or more blood samples 102 may be collected from one or more blood banks. Accordingly, in some embodiments, blood samples 102 may be screened through use of one or more separation channels 118. In some embodiments, one or more blood samples 102 may be collected from a patient. Accordingly, in some embodiments, blood samples 102 may be used for diagnostic purposes. In some examples, blood samples 102 may be used to detect drug use by an individual.

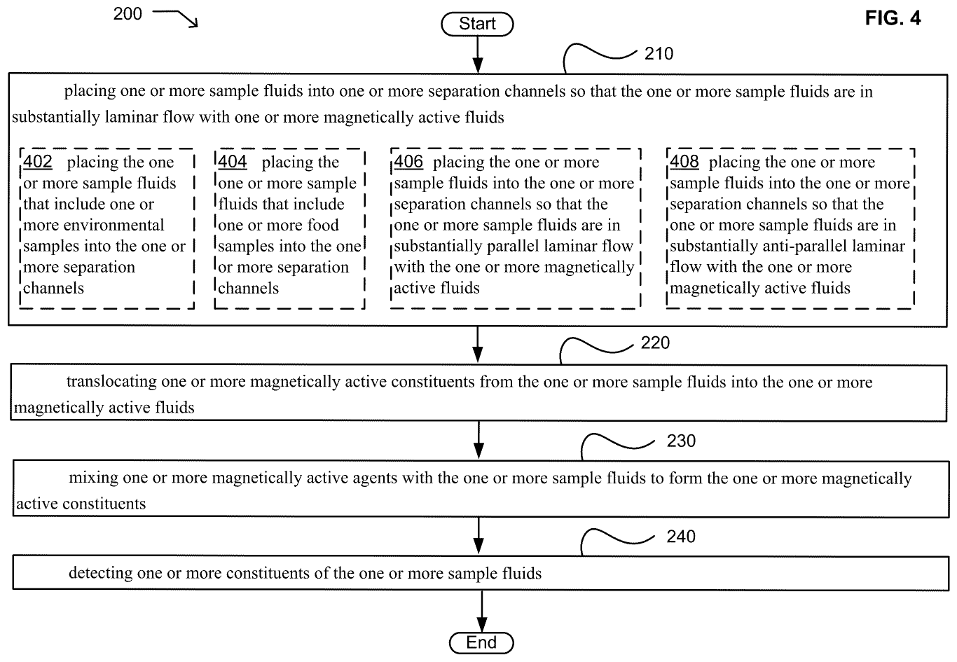

FIG. 4 illustrates alternative embodiments of the example operational flow 200 of FIG. 2. FIG. 4 illustrates example embodiments where the placing operation 210 may include at least one additional operation. Additional operations may include an operation 402, an operation 404, an operation 406, and/or an operation 408.

At operation 402, the placing operation 210 may include placing the one or more sample fluids that include one or more environmental samples into the one or more separation channels. In some embodiments, one or more sample fluids 104 that include one or more environmental samples 102 may be placed into one or more separation channels 118. Numerous types of environmental samples 102 may be placed into one or more separation channels 118. Examples of such environmental samples 102 include, but are not limited to, soil samples 102, water samples 102, plant samples 102, farm related samples 102, industrial samples 102, fishery related samples 102, and the like. In some embodiments, environmental samples 102 may include gas (e.g., air) samples 102 that have been filtered through a fluid.

At operation 404, the placing operation 210 may include placing the one or more sample fluids that include one or more food samples into the one or more separation channels. In some embodiments, one or more sample fluids 104 that include one or more food samples 102 may be placed into one or more separation channels 118. Numerous types of food samples 102 may be placed into one or more separation channels 118. Examples of such food samples 102 include, but are not limited to, vegetables, meats, cheeses, juices, milk, fruits, nuts, prepared foods, raw foods, and the like. Accordingly, components of one or more food samples 102 may be separated through use of one or more separation channels 118. In some embodiments, allergens may be separated from one or more food samples 102 (e.g., U.S. patent application Ser. Nos. 11/699,770, 11/699,920, 11/699,747, 11/699,774, herein incorporated by reference). In some embodiments, pathogens may be separated from one or more food samples 102 (e.g., U.S. patent application Ser. Nos. 11/729,301, 11/729,274, 11/729,276, herein incorporated by reference).

At operation 406, the placing operation 210 may include placing the one or more sample fluids into the one or more separation channels so that the one or more sample fluids are in substantially parallel laminar flow with the one or more magnetically active fluids. In some embodiments, one or more sample fluids 104 may be placed into one or more separation channels 118 so that the one or more sample fluids 104 are in substantially parallel laminar flow with one or more magnetically active fluids 126. In such embodiments, the one or more sample fluids 104 flow in the same direction as the one or more magnetically active fluids 126. In some embodiments, sample fluids 104 and magnetically active fluids 126 may be matched to each other. For example, in some embodiments, sample fluids 104 and magnetically active fluids 126 may be selected that are immiscible with each other. Accordingly, numerous characteristics may be considered when selecting sample fluids 104 and magnetically active fluids. Examples of such characteristics include, but are not limited to, viscosity, density, miscibility, solubility, vapor pressure, and the like. In some embodiments, the one or more magnetically active fluids 126 may include a ferrofluid. In some embodiments, the one or more magnetically active fluids 126 may include paramagnetic particles. In some embodiments, the one or more magnetically active fluids 126 may include diamagnetic particles. In some embodiments, the one or more magnetically active fluids 126 may include magnetic particles.

At operation 408, the placing operation 210 may include placing the one or more sample fluids into the one or more separation channels so that the one or more sample fluids are in substantially anti-parallel laminar flow with the one or more magnetically active fluids. In some embodiments, one or more sample fluids 104 may be placed into one or more separation channels 118 so that the one or more sample fluids 104 are in substantially anti-parallel laminar flow with one or more magnetically active fluids. In such embodiments, the one or more sample fluids 104 flow in the opposite direction as the one or more magnetically active fluids. In some embodiments, sample fluids 104 and magnetically active fluids 126 may be matched to each other. For example, in some embodiments, sample fluids 104 and magnetically active fluids 126 may be selected that are immiscible with each other. Accordingly, numerous characteristics may be considered when selecting sample fluids 104 and magnetically active fluids 126. Examples of such characteristics include, but are not limited to, viscosity, density, miscibility, solubility, vapor pressure, and the like. In some embodiments, the one or more magnetically active fluids 126 may include a ferrofluid. In some embodiments, the one or more magnetically active fluids 126 may include paramagnetic particles. In some embodiments, the one or more magnetically active fluids 126 may include diamagnetic particles. In some embodiments, the one or more magnetically active fluids 126 may include magnetic particles.

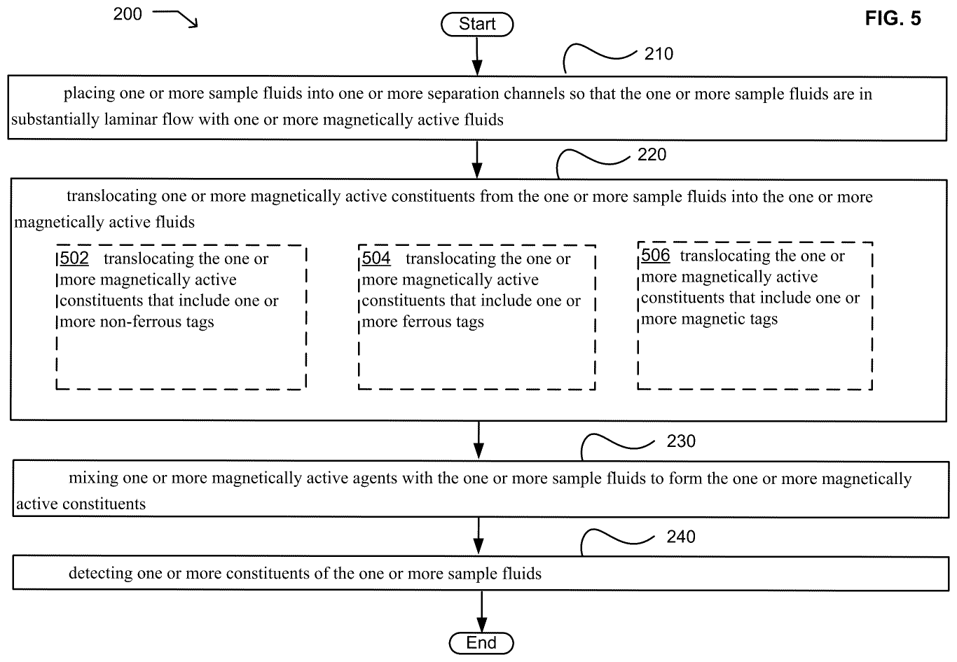

FIG. 5 illustrates alternative embodiments of the example operational flow 200 of FIG. 2. FIG. 5 illustrates example embodiments where the translocating operation 220 may include at least one additional operation. Additional operations may include an operation 502, an operation 504, and/or an operation 506.

At operation 502, the translocating operation 220 may include translocating the one or more magnetically active constituents that include one or more non-ferrous tags. In some embodiments, one or more magnetically active constituents 106 that include one or more non-ferrous tags may be translocated. Numerous types of non-ferrous tags may be transported. In some embodiments, non-ferrous tags may be magnetic. Examples of non-ferrous permanent magnets 124 include, but are not limited to, alnico magnets 124, samarium-cobalt magnets 124, plastic magnets 124, and the like. In some embodiments, non-ferrous tags may be paramagnetic. In some embodiments, non-ferrous tags may be diamagnetic. In some embodiments, non-ferrous tags and magnetically active fluids 126 may be matched to each other. For example, in some embodiments, a non-ferrous tag that is a permanent magnet 124 may be matched with a magnetically active fluid 126 to which the tag is attracted to facilitate translocation of the tag. In some embodiments, a non-ferrous tag that is paramagnetic may be matched with a magnetically active fluid 126 to which the tag is attracted to facilitate translocation of the tag. In some embodiments, a non-ferrous tag may be selected that is repelled by a permanent magnet 124.

At operation 504, the translocating operation 220 may include translocating the one or more magnetically active constituents that include one or more ferrous tags.

In some embodiments, one or more magnetically active constituents 106 that include one or more ferrous tags may be translocated. Numerous types of ferrous tags may be transported. In some embodiments, ferrous tags may be magnetic. Examples of ferrous permanent magnets 124 include, but are not limited to, neodymium magnets 124, ceramic magnets 124, ferromagnets 124, and the like. In some embodiments, ferrous tags and magnetically active fluids 126 may be matched to each other. For example, in some embodiments, a ferrous tag that is a permanent magnet 124 may be matched with a magnetically active fluid 126 to which the tag is attracted to facilitate translocation of the tag. In some embodiments, a ferrous tag that is paramagnetic may be matched with a magnetically active fluid 126 to which the tag is attracted to facilitate translocation of the tag.

At operation 506, the translocating operation 220 may include translocating the one or more magnetically active constituents that include one or more magnetic tags. In some embodiments, one or more magnetically active constituents 106 that include one or more magnetic tags may be translocated. Numerous types of magnetic tags may be transported. Examples of magnetic tags include, but are not limited to, neodymium magnets, ceramic magnets, ferromagnets, alnico magnets, samarium-cobalt magnets, plastic magnets, and the like. In some embodiments, magnetic tags and magnetically active fluids 126 may be matched to each other. For example, in some embodiments, a magnetic tag that is a permanent magnet 124 may be matched with a magnetically active fluid 126 to which the tag is attracted to facilitate translocation of the tag.

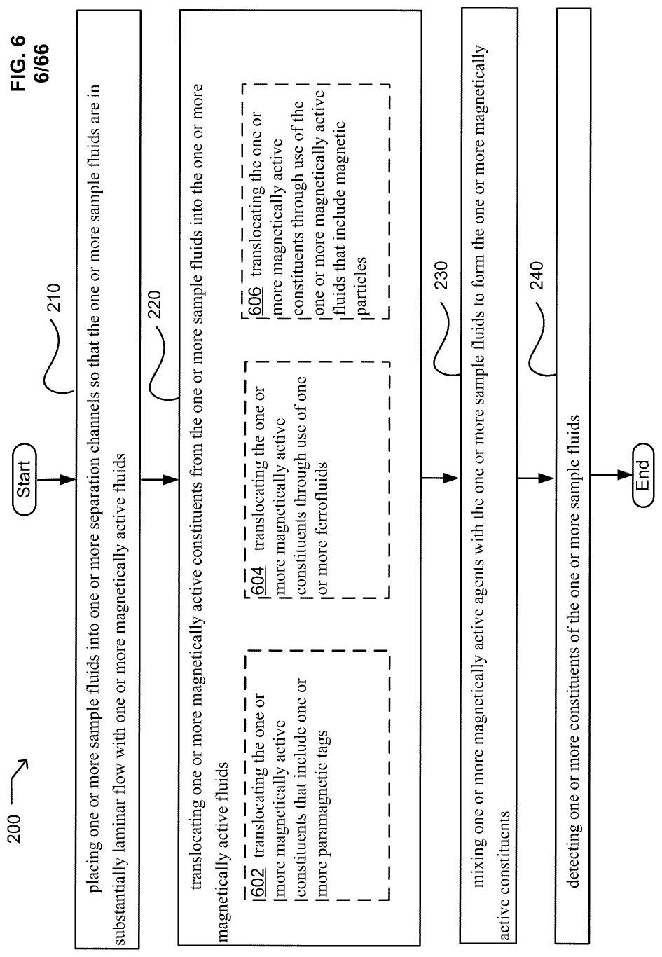

FIG. 6 illustrates alternative embodiments of the example operational flow 200 of FIG. 2. FIG. 6 illustrates example embodiments where the translocating operation 220 may include at least one additional operation. Additional operations may include an operation 602, an operation 604, and/or an operation 606.

At operation 602, the translocating operation 220 may include translocating the one or more magnetically active constituents that include one or more paramagnetic tags.

In some embodiments, one or more magnetically active constituents 106 that include one or more paramagnetic tags may be translocated. Numerous types of paramagnetic tags may be transported. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like. In some embodiments, paramagnetic tags and magnetically active fluids 126 may be matched to each other. For example, in some embodiments, a paramagnetic tag may be matched with a magnetically active fluid 126 to which the tag is attracted to facilitate translocation of the tag.

At operation 604, the translocating operation 220 may include translocating the one or more magnetically active constituents through use of one or more ferrofluids. In some embodiments, one or more magnetically active constituents 106 may be translocated through use of one or more ferrofluids. In some embodiments, a magnetically active constituent 106 may include a magnet 124 that is attracted to one or more ferrofluids and thereby facilitates translocation of the magnetically active constitutent 106 into the ferrofluid.

At operation 606, the translocating operation 220 may include translocating the one or more magnetically active constituents through use of the one or more magnetically active fluids that include magnetic particles. In some embodiments, one or more magnetically active constituents 106 may be translocated through use of one or more magnetically active fluids 126 that include magnetic particles. In some embodiments, the magnetic particles may be coated with a surfactant. Examples of such surfactants include, but are not limited to, oleic acid, tetramethylammonium hydroxide, citric acid, soy lecithin, and the like.

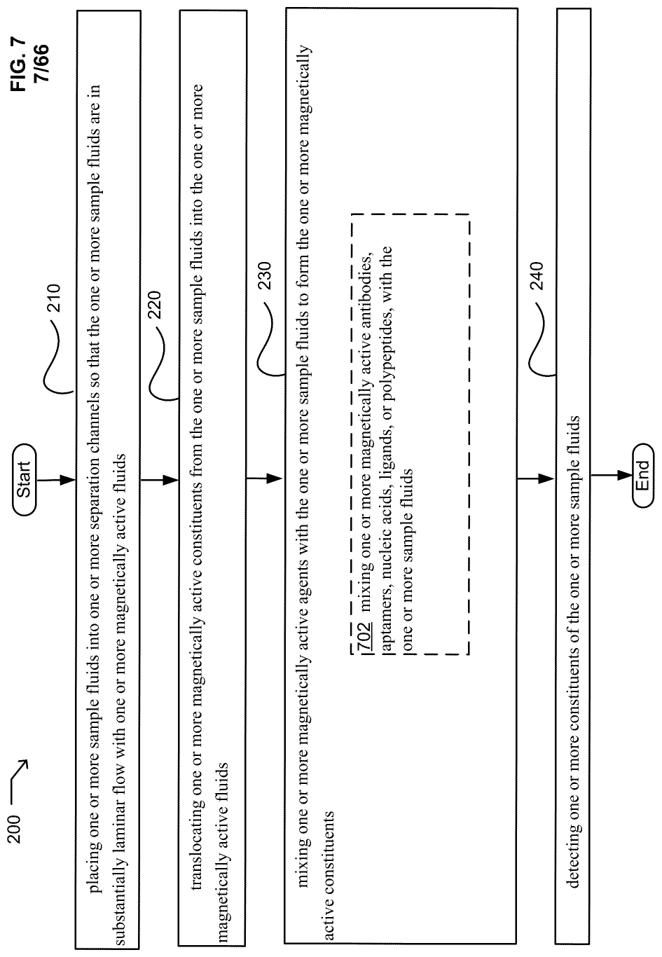

FIG. 7 illustrates alternative embodiments of the example operational flow 200 of FIG. 2. FIG. 7 illustrates example embodiments where the mixing operation 230 may include at least one additional operation. Additional operations may include an operation 702.

At operation 702, the mixing operation 230 may include mixing one or more magnetically active antibodies, aptamers, nucleic acids, ligands, or polypeptides, with the one or more sample fluids. In some embodiments, one or more magnetically active antibodies, aptamers, nucleic acids, ligands, polypeptides, or substantially any combination thereof, may be mixed with one or more sample fluids 104. In some embodiments, one or more magnetically active antibodies, aptamers, nucleic acids, ligands, or polypeptides, or substantially any combination thereof, may be mixed with one or more sample fluids 104 to form one or more magnetically active constituents 106. In some embodiments, the one or more magnetically active antibodies, aptamers, nucleic acids, ligands, or polypeptides may include a magnetically active tag. In some embodiments, the magnetically active tag may be a permanent magnet 124. In some embodiments, the magnetically active tag may be paramagnetic. In some embodiments, the magnetically active tag may be diamagnetic.

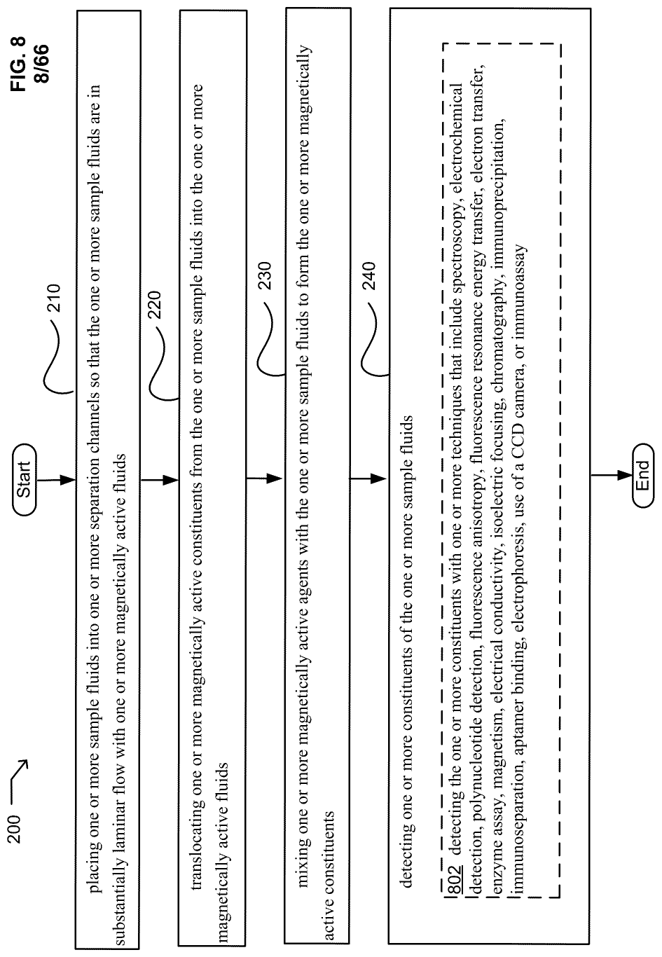

FIG. 8 illustrates alternative embodiments of the example operational flow 200 of FIG. 2. FIG. 8 illustrates example embodiments where the detecting operation 240 may include at least one additional operation. Additional operations may include an operation 802.

At operation 802, the detecting operation 240 may include detecting the one or more constituents with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, or immunoassay. In some embodiments, one or more constituents of one or more samples 102 may be detected with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, immunoassay, or substantially any combination thereof. Such methods have been described (e.g., U.S. patent application Ser. Nos. 11/699,770, 11/699,920, 11/699,747, 11/699,774, 11/729,301, 11/729,274, and 11/729,276; herein incorporated by reference). In some embodiments, one or more separation channels 118 may be operably coupled to one or more detection chambers. In some embodiments, one or more detection units 130 may facilitate detection of one or more constituents.

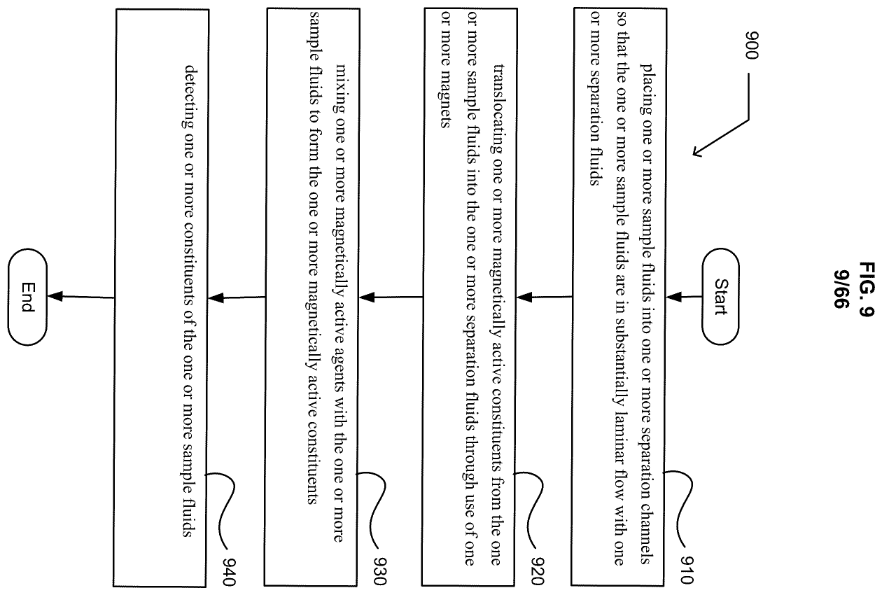

FIG. 9 illustrates an operational flow 900 representing examples of operations that are related to the performance of a method that may be used to separate one or more constituents from one or more samples 102. In FIG. 9 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 900 includes a placing operation 910 involving placing one or more sample fluids into one or more separation channels so that the one or more sample fluids are in substantially laminar flow with one or more separation fluids. In some embodiments, placing operation 910 may include suspending one or more samples in one or more fluids to form the one or more sample fluids. In some embodiments, placing operation 910 may include placing the one or more sample fluids that include one or more bodily samples into the one or more separation channels. In some embodiments, placing operation 910 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, placing operation 910 may include placing the one or more sample fluids that include blood into the one or more separation channels. In some embodiments, placing operation 910 may include placing the one or more sample fluids that include one or more environmental samples into the one or more separation channels. In some embodiments, placing operation 910 may include placing the one or more sample fluids that include one or more food samples into the one or more separation channels. In some embodiments, placing operation 910 may include placing the one or more sample fluids into the one or more separation channels so that the one or more sample fluids are in substantially parallel laminar flow with the one or more separation fluids. In some embodiments, placing operation 910 may include placing the one or more sample fluids into the one or more separation channels so that the one or more sample fluids are in substantially anti-parallel laminar flow with the one or more separation fluids.

After a start operation, the operational flow 900 includes a translocating operation 920 involving translocating one or more magnetically active constituents from the one or more sample fluids into the one or more separation fluids through use of one or more magnets. In some embodiments, translocating operation 920 may include translocating the one or more magnetically active constituents that include one or more non-ferrous tags. In some embodiments, translocating operation 920 may include translocating the one or more magnetically active constituents that include one or more ferrous tags. In some embodiments, translocating operation 920 may include translocating the one or more magnetically active constituents that include one or more magnetic tags. In some embodiments, translocating operation 920 may include translocating the one or more magnetically active constituents that include one or more paramagnetic tags. In some embodiments, translocating operation 920 may include translocating the one or more magnetically active constituents through use of magnetic attraction. In some embodiments, translocating operation 920 may include translocating the one or more magnetically active constituents through use of magnetic repulsion. In some embodiments, translocating operation 920 may include translocating the one or more magnetically active constituents through use of one or more eddy currents.

After a start operation, the operational flow 900 may optionally include a mixing operation 930 involving mixing one or more magnetically active agents with the one or more sample fluids to form the one or more magnetically active constituents. In some embodiments, mixing operation 930 may include mixing one or more magnetically active antibodies, aptamers, nucleic acids, ligands, or polypeptides, with the one or more sample fluids.

After a start operation, the operational flow 900 may optionally include a detecting operation 940 involving detecting one or more constituents of the one or more sample fluids. In some embodiments, detecting operation 940 may include detecting the one or more constituents with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, or immunoassay.

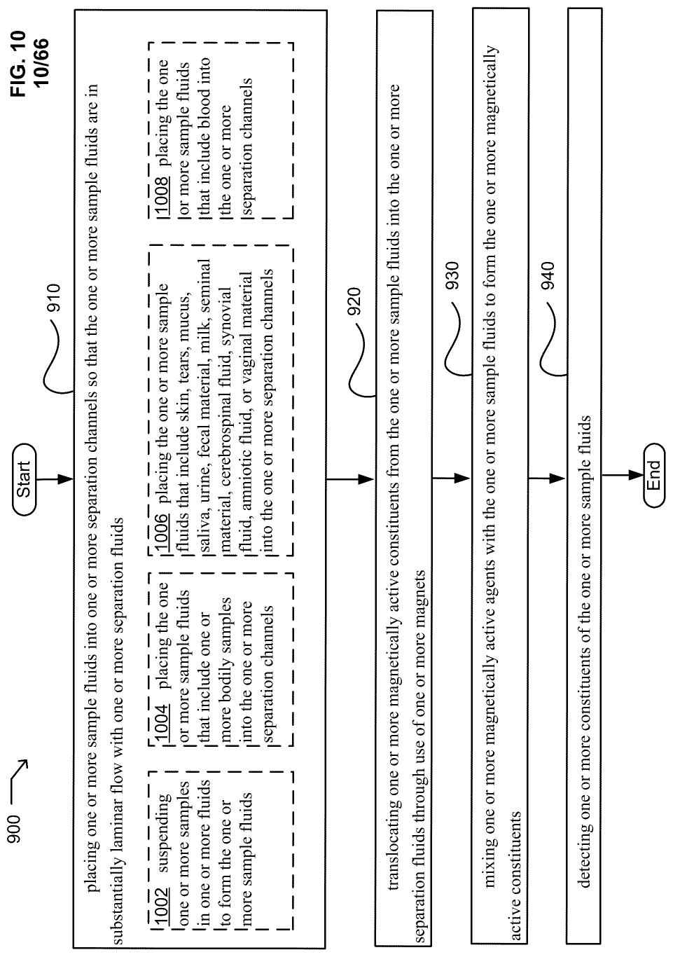

FIG. 10 illustrates alternative embodiments of the example operational flow 900 of FIG. 9. FIG. 10 illustrates example embodiments where the placing operation 910 may include at least one additional operation. Additional operations may include an operation 1002, an operation 1004, an operation 1006, and/or an operation 1008.

At operation 1002, the placing operation 910 may include suspending one or more samples in one or more fluids to form the one or more sample fluids. In some embodiments, one or more samples 102 may be suspended in one or more fluids to form one or more sample fluids 104. In some embodiments, one or more samples 102 may be dissolved in one or more solvents. Numerous types of samples 102 may be suspended in one or more fluids to form one or more sample fluids 104. Examples of samples 102 include, but are not limited to, solid samples 102, liquid samples 102, semi-solid samples 102, gels, and the like. Such samples 102 may include, but are not limited to, food samples 102, biological samples 102, fuel samples 102, environmental samples 102, crop samples 102, water samples 102, diagnostic samples 102 (e.g., tissue, blood, saliva, mucus, cerebrospinal fluid, amniotic fluid, and the like). In some embodiments, blood samples 102 may be combined with one or more fluids to form one or more sample fluids 104. In some embodiments, one or more fluids may include desired functions. For example, in some embodiments, one or more fluids that include nuclease inhibitors may be mixed with one or more samples 102. In some embodiments, one or more fluids that include one or more RNase inhibitors may be mixed with one or more blood samples 102 to preserve polyribonucleic acids present within the one or more blood samples 102. In some embodiments, precipitates may be suspended in one or more fluids. For example, in some embodiments, precipitated polynucleotides may be suspended in one or more fluids to form one or more sample fluids 104. In some embodiments, one or more fluids may be used to extract one or more components from a first sample 102. In some embodiments, one or more fluids may be selected to exhibit desired fluid characteristics. Examples of such characteristics include, but are not limited to, viscosity, density, miscibility, solubility, polarity, vapor pressure, flammability, and the like. Accordingly, numerous types of samples 102 and numerous types of fluids may be used to prepare a sample fluid 104.

At operation 1004, the placing operation 910 may include placing the one or more sample fluids that include one or more bodily samples into the one or more separation channels. In some embodiments, one or more sample fluids 104 that include one or more bodily samples 102 may be placed into one or more separation channels 118. Examples of such bodily samples 102 include, but are not limited to, tissue, tears, saliva, mucus, wax, blood, synovial fluid, cerebrospinal fluid, seminal fluid, vaginal fluid, amniotic fluid, urine, fecal material, and the like. In some embodiments, such samples 102 may be used within diagnostic methods. For example, in some embodiments, amniotic fluid may be used to determine if a fetus exhibits a genotype associated with a disease. In other examples, parasites within fecal material may be detected to determine if an individual is infected with a parasite. Numerous pathogens and parasites have been described (e.g., U.S. patent application Ser. Nos. 11/729,301, 11/729,274, and 11/729,276, herein incorporated by reference). Accordingly, numerous types of bodily samples 102 may be selected.

At operation 1006, the placing operation 910 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, one or more sample fluids 104 that include skin, tears, mucus, saliva, urine, fecal material, milk, seminal material, cerebrospinal fluid, synovial fluid, amniotic fluid, vaginal material, or the like may be placed into one or more separation channels 118 in substantially any combination.

At operation 1008, the placing operation 910 may include placing the one or more sample fluids that include blood into the one or more separation channels. In some embodiments, one or more sample fluids 104 that include blood may be placed into one or more separation channels 118. In some embodiments, one or more blood samples 102 may be collected from one or more blood banks. Accordingly, in some embodiments, blood samples 102 may be screened through use of one or more separation channels 118. In some embodiments, one or more blood samples 102 may be collected from a patient. Accordingly, in some embodiments, blood samples 102 may be used for diagnostic purposes. In some examples, blood samples 102 may be used to detect drug use by an individual.

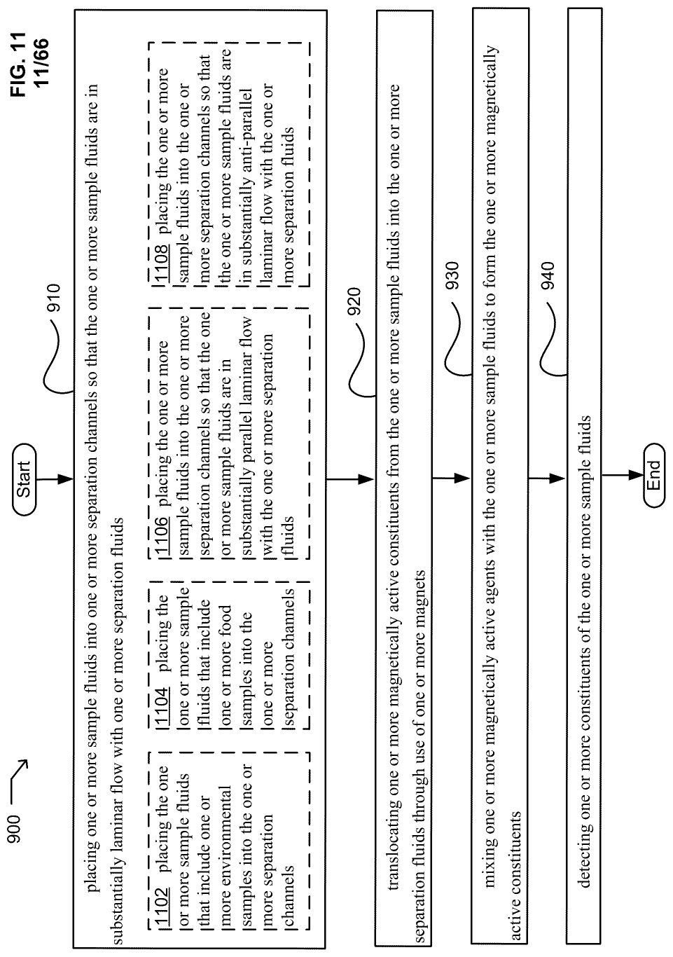

FIG. 11 illustrates alternative embodiments of the example operational flow 900 of FIG. 9. FIG. 11 illustrates example embodiments where the placing operation 910 may include at least one additional operation. Additional operations may include an operation 1102, an operation 1104, an operation 1106, and/or an operation 1108.

At operation 1102, the placing operation 910 may include placing the one or more sample fluids that include one or more environmental samples into the one or more separation channels. In some embodiments, one or more sample fluids 104 that include one or more environmental samples 102 may be placed into one or more separation channels 118. Numerous types of environmental samples 102 may be placed into one or more separation channels 118. Examples of such environmental samples 102 include, but are not limited to, soil samples 102, water samples 102, plant samples 102, farm related samples 102, industrial samples 102, fishery related samples 102, and the like. In some embodiments, environmental samples 102 may include gas (e.g., air) samples 102 that have been filtered through a fluid.

At operation 1104, the placing operation 910 may include placing the one or more sample fluids that include one or more food samples into the one or more separation channels. In some embodiments, one or more sample fluids 104 that include one or more food samples 102 may be placed into one or more separation channels 118. Numerous types of food samples 102 may be placed into one or more separation channels 118. Examples of such food samples 102 include, but are not limited to, vegetables, meats, cheeses, juices, milk, fruits, nuts, prepared foods, raw foods, and the like. Accordingly, components of one or more food samples 102 may be separated through use of one or more separation channels 118. In some embodiments, allergens may be separated from one or more food samples 102 (e.g., U.S. patent application Ser. Nos. 11/699,770, 11/699,920, 11/699,747, 11/699,774, herein incorporated by reference). In some embodiments, pathogens may be separated from one or more food samples 102 (e.g., U.S. patent application Ser. Nos. 11/729,301, 11/729,274, 11/729,276, herein incorporated by reference).

At operation 1106, the placing operation 910 may include placing the one or more sample fluids into the one or more separation channels so that the one or more sample fluids are in substantially parallel laminar flow with the one or more separation fluids. In some embodiments, one or more sample fluids 104 may be placed into one or more separation channels 118 so that the one or more sample fluids 104 are in substantially parallel laminar flow with one or more magnetically active fluids 126. In such embodiments, the one or more sample fluids 104 flow in the same direction as the one or more magnetically active fluids 126. In some embodiments, sample fluids 104 and magnetically active fluids 126 may be matched to each other. For example, in some embodiments, sample fluids 104 and magnetically active fluids 126 may be selected that are immiscible with each other. Accordingly, numerous characteristics may be considered when selecting sample fluids 104 and magnetically active fluids 126. Examples of such characteristics include, but are not limited to, viscosity, density, miscibility, solubility, vapor pressure, and the like. In some embodiments, the one or more magnetically active fluids 126 may include a ferrofluid. In some embodiments, the one or more magnetically active fluids 126 may include paramagnetic particles. In some embodiments, the one or more magnetically active fluids 126 may include diamagnetic particles. In some embodiments, the one or more magnetically active fluids 126 may include magnetic particles.

At operation 1108, the placing operation 910 may include placing the one or more sample fluids into the one or more separation channels so that the one or more sample fluids are in substantially anti-parallel laminar flow with the one or more separation fluids. In some embodiments, one or more sample fluids 104 may be placed into one or more separation channels 118 so that the one or more sample fluids 104 are in substantially anti-parallel laminar flow with one or more magnetically active fluids 126. In such embodiments, the one or more sample fluids 104 flow in the opposite direction as the one or more magnetically active fluids 126. In some embodiments, sample fluids 104 and magnetically active fluids 126 may be matched to each other. For example, in some embodiments, sample fluids 104 and magnetically active fluids 126 may be selected that are immiscible with each other. Accordingly, numerous characteristics may be considered when selecting sample fluids 104 and magnetically active fluids 126. Examples of such characteristics include, but are not limited to, viscosity, density, miscibility, solubility, vapor pressure, and the like. In some embodiments, the one or more magnetically active fluids 126 may include a ferrofluid. In some embodiments, the one or more magnetically active fluids 126 may include paramagnetic particles. In some embodiments, the one or more magnetically active fluids 126 may include diamagnetic particles. In some embodiments, the one or more magnetically active fluids 126 may include magnetic particles.

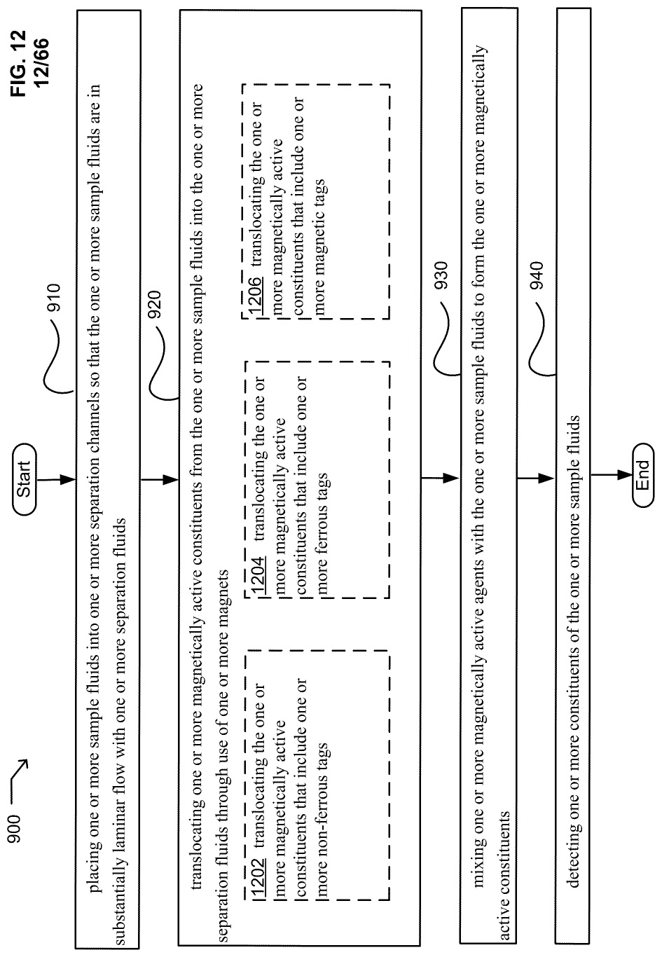

FIG. 12 illustrates alternative embodiments of the example operational flow 900 of FIG. 9. FIG. 12 illustrates example embodiments where the translocating operation 920 may include at least one additional operation. Additional operations may include an operation 1202, an operation 1204, and/or an operation 1206.

At operation 1202, the translocating operation 920 may include translocating the one or more magnetically active constituents that include one or more non-ferrous tags. In some embodiments, one or more magnetically active constituents 106 that include one or more non-ferrous tags may be translocated. Numerous types of non-ferrous tags may be transported. In some embodiments, non-ferrous tags may be magnetic. Examples of non-ferrous permanent magnets 124 include, but are not limited to, alnico magnets 124, samarium-cobalt magnets 124, plastic magnets 124, and the like. In some embodiments, non-ferrous tags may be paramagnetic. In some embodiments, non-ferrous tags may be diamagnetic. In some embodiments, non-ferrous tags and magnetically active fluids 126 may be matched to each other. For example, in some embodiments, a non-ferrous tag that is a permanent magnet 124 may be matched with a magnetically active fluid 126 to which the tag is attracted to facilitate translocation of the tag. In some embodiments, a non-ferrous tag that is paramagnetic may be matched with a magnetically active fluid 126 to which the tag is attracted to facilitate translocation of the tag. In some embodiments, a non-ferrous tag may be selected that is repelled by a permanent magnet 124.

At operation 1204, the translocating operation 920 may include translocating the one or more magnetically active constituents that include one or more ferrous tags. In some embodiments, one or more magnetically active constituents 106 that include one or more ferrous tags may be translocated. Numerous types of ferrous tags may be transported. In some embodiments, ferrous tags may be magnetic. Examples of ferrous permanent magnets 124 include, but are not limited to, neodymium magnets 124, ceramic magnets 124, ferromagnets 124, and the like. In some embodiments, ferrous tags and magnetically active fluids 126 may be matched to each other. For example, in some embodiments, a ferrous tag that is a permanent magnet 124 may be matched with a magnetically active fluid 126 to which the tag is attracted to facilitate translocation of the tag. In some embodiments, a ferrous tag that is paramagnetic may be matched with a magnetically active fluid 126 to which the tag is attracted to facilitate translocation of the tag.

At operation 1206, the translocating operation 920 may include translocating the one or more magnetically active constituents that include one or more magnetic tags. In some embodiments, one or more magnetically active constituents 106 that include one or more magnetic tags may be translocated. Numerous types of magnetic tags may be transported. Examples of magnetic tags include, but are not limited to, neodymium magnets, ceramic magnets, ferromagnets, alnico magnets, samarium-cobalt magnets, plastic magnets, and the like. In some embodiments, magnetic tags and magnetically active fluids 126 may be matched to each other. For example, in some embodiments, a magnetic tag that is a permanent magnet 124 may be matched with a magnetically active fluid 126 to which the tag is attracted to facilitate translocation of the tag.

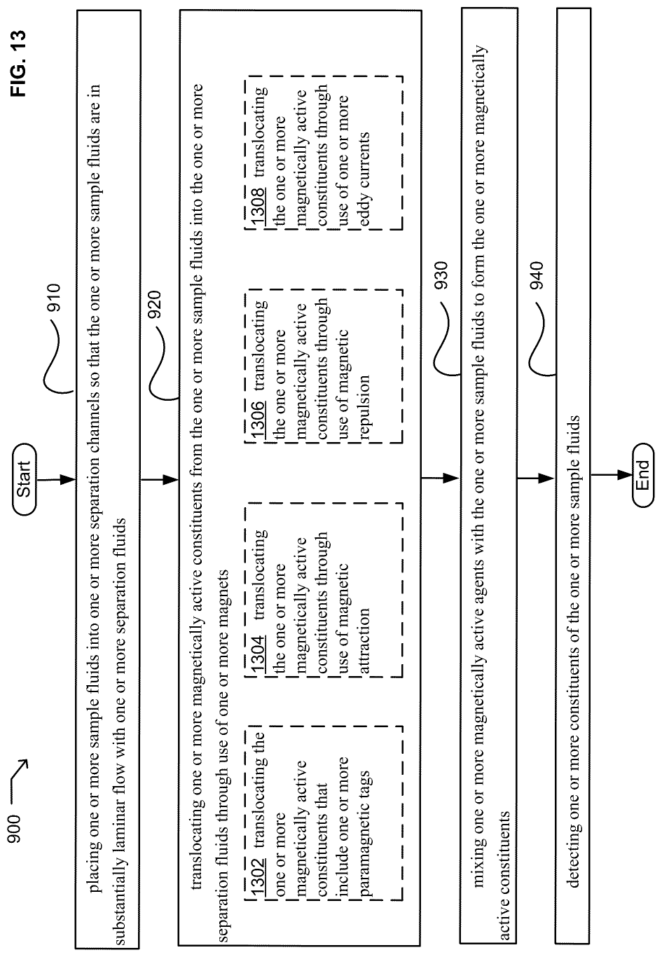

FIG. 13 illustrates alternative embodiments of the example operational flow 900 of FIG. 9. FIG. 13 illustrates example embodiments where the translocating operation 920 may include at least one additional operation. Additional operations may include an operation 1302, an operation 1304, an operation 1306, and/or an operation 1308.

At operation 1302, the translocating operation 920 may include translocating the one or more magnetically active constituents that include one or more paramagnetic tags. In some embodiments, one or more magnetically active constituents 106 that include one or more paramagnetic tags may be translocated. Numerous types of paramagnetic tags may be transported. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like. In some embodiments, paramagnetic tags and magnetically active fluids 126 may be matched to each other. For example, in some embodiments, a paramagnetic tag may be matched with a magnetically active fluid 126 to which the tag is attracted to facilitate translocation of the tag. In some embodiments, one or more magnets 124 may be used to facilitate translocation of one or more magnetically active constituents 106.

At operation 1304, the translocating operation 920 may include translocating the one or more magnetically active constituents through use of magnetic attraction. In some embodiments, one or more magnetically active constituents 106 may be translocated through use of magnetic attraction. For example, in some embodiments, one or more magnetically active constituents 106 may be translocated from one or more sample fluids 104 to one or more separation fluids through use of one or more magnets 124. In some embodiments, the one or more magnets 124 may include one or more permanent magnets 124. In some embodiments, the one or more magnets 124 may include one or more electromagnets 124.

At operation 1306, the translocating operation 920 may include translocating the one or more magnetically active constituents through use of magnetic repulsion. In some embodiments, one or more magnetically active constituents 106 may be translocated through use of magnetic repulsion. For example, in some embodiments, one or more constituents that are inherently diamagnetic may be translocated from one or more sample fluids 104 to one or more separation fluids through use of one or more magnets. Examples of constituents that are inherently diamagnetic include, but are not limited to, polynucleic acids (e.g., deoxyribonucleic acid), organic compounds (e.g., oil, plastic, pyrolytic carbon), metals (e.g., mercury, gold, bismuth), and the like. Accordingly, in some embodiments, organic compounds, metals, biological materials, may be separated from one or more samples 102 through use of magnetic repulsion. In some embodiments, one or more magnetically active constituents 106 that include one or more diamagnetic tags may be translocated from one or more sample fluids 104 to one or more separation fluids through use of magnetic repulsion. For example, in some embodiments, one or more constituents within one or more samples 102 may be mixed with one or more antibodies that are coupled to a diamagnetic tag to form a magnetically active constituent 106 that may be separated through magnetic repulsion.

At operation 1308, the translocating operation 920 may include translocating the one or more magnetically active constituents through use of one or more eddy currents. In some embodiments, one or more magnetically active constituents 106 may be translocated through use of one or more eddy currents. For example, in some embodiments, one or more magnetically active constituents 106 may be translocated from one or more sample fluids 104 to one or more separation fluids through use of one or more eddy currents. In some embodiments, one or more eddy currents may be created through use of one or more permanent magnets. In some embodiments, one or more eddy currents may be created through use of one or more electromagnets. In some embodiments, non-ferrous metals may be separated from one or more sample fluids 104 through use of eddy currents.

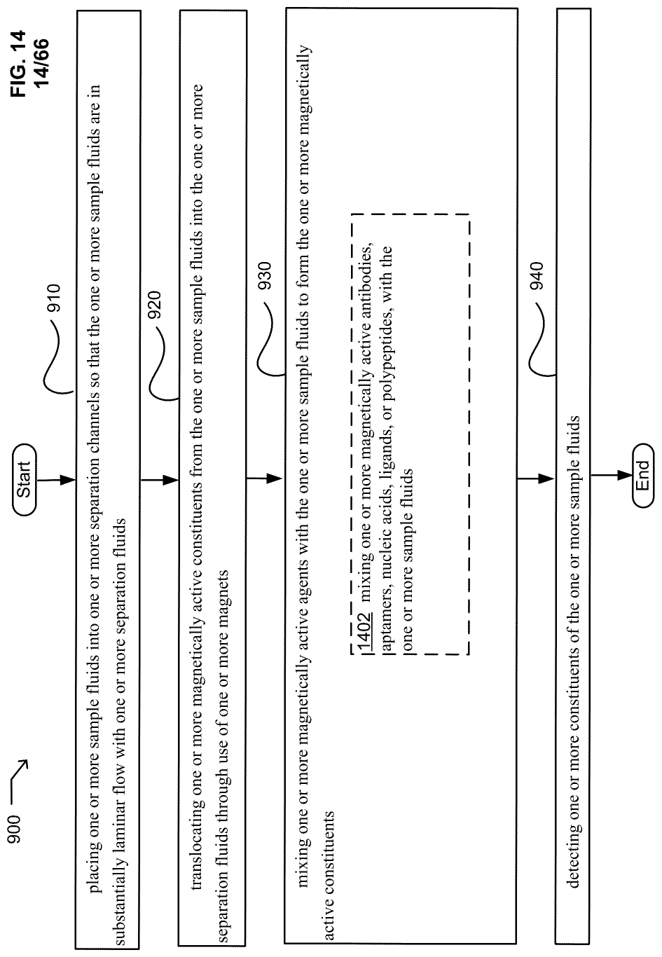

FIG. 14 illustrates alternative embodiments of the example operational flow 900 of FIG. 9. FIG. 14 illustrates example embodiments where the mixing operation 930 may include at least one additional operation. Additional operations may include an operation 1402.

At operation 1402, the mixing operation 930 may include mixing one or more magnetically active antibodies, aptamers, nucleic acids, ligands, or polypeptides, with the one or more sample fluids. In some embodiments, one or more magnetically active antibodies, aptamers, nucleic acids, ligands, polypeptides, or substantially any combination thereof, may be mixed with one or more sample fluids 104. In some embodiments, one or more magnetically active antibodies, aptamers, nucleic acids, ligands, or polypeptides, or substantially any combination thereof, may be mixed with one or more sample fluids 104 to form one or more magnetically active constituents 106. In some embodiments, the one or more magnetically active antibodies, aptamers, nucleic acids, ligands, or polypeptides may include a magnetically active tag. In some embodiments, the magnetically active tag may be a permanent magnet 124. In some embodiments, the magnetically active tag may be paramagnetic. In some embodiments, the magnetically active tag may be diamagnetic.

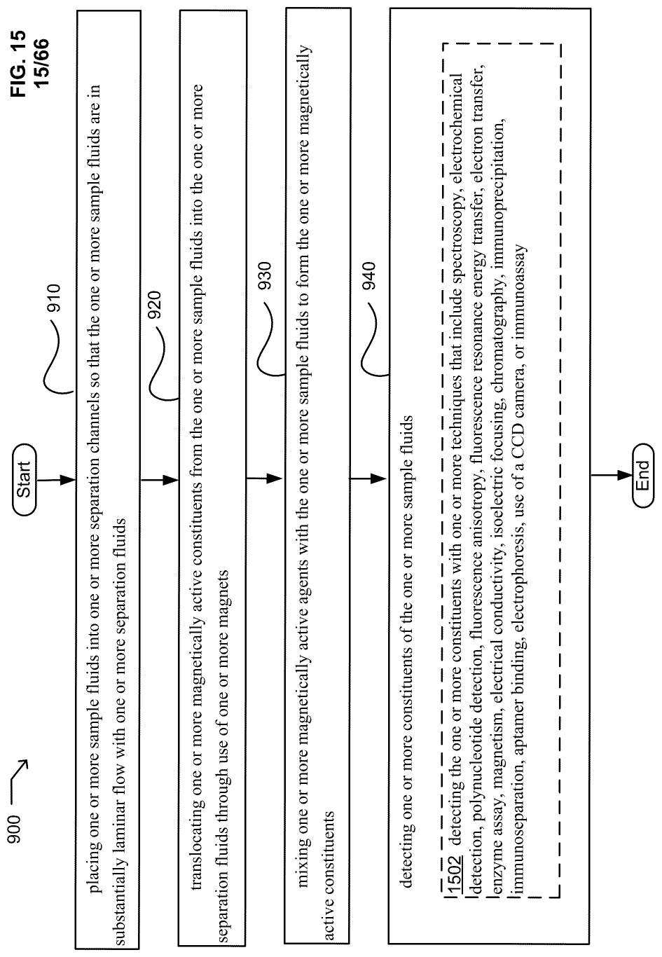

FIG. 15 illustrates alternative embodiments of the example operational flow 900 of FIG. 9. FIG. 15 illustrates example embodiments where the detecting operation 940 may include at least one additional operation. Additional operations may include an operation 1502.

At operation 1502, the detecting operation 940 may include detecting the one or more constituents with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, or immunoassay. In some embodiments, one or more constituents of one or more samples 102 may be detected with one or more techniques that include spectroscopy, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, electrophoresis, use of a CCD camera, immunoassay, or substantially any combination thereof. Such methods have been described (e.g., U.S. patent application Ser. Nos. 11/729,301, 11/729,274, 11/729,276, 11/699,770, 11/699,920, 11/699,747, and 11/699,774; herein incorporated by reference). In some embodiments, one or more separation channels 118 may be operably coupled to one or more detection chambers. In some embodiments, one or more detection units 130 may facilitate detection of one or more constituents.

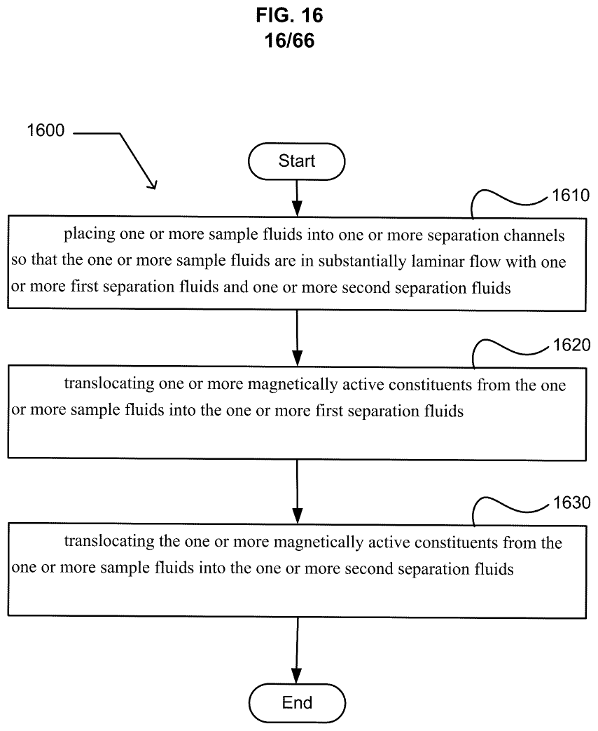

FIG. 16 illustrates an operational flow 1600 representing examples of operations that are related to the performance of a method that may be used to separate one or more constituents from one or more samples 102. In FIG. 16 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 1600 includes a placing operation 1610 involving placing one or more sample fluids into one or more separation channels so that the one or more sample fluids are in substantially laminar flow with one or more first separation fluids and one or more second separation fluids. In some embodiments, placing operation 1610 may include suspending one or more samples in one or more fluids to form the one or more sample fluids. In some embodiments, placing operation 1610 may include placing the one or more sample fluids that include one or more bodily samples into the one or more separation channels. In some embodiments, placing operation 1610 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, placing operation 1610 may include placing the one or more sample fluids that include blood into the one or more separation channels. In some embodiments, placing operation 1610 may include placing the one or more sample fluids that include one or more environmental samples into the one or more separation channels. In some embodiments, placing operation 1610 may include placing the one or more sample fluids that include one or more food samples into the one or more separation channels. In some embodiments, placing operation 1610 may include placing the one or more sample fluids into the one or more separation channels so that the one or more sample fluids are in substantially parallel laminar flow with the one or more first separation fluids. In some embodiments, placing operation 1610 may include placing the one or more sample fluids into the one or more separation channels so that the one or more sample fluids are in substantially anti-parallel laminar flow with the one or more first separation fluids. In some embodiments, placing operation 1610 may include placing the one or more sample fluids into the one or more separation channels so that the one or more sample fluids are in substantially parallel laminar flow with the one or more second separation fluids. In some embodiments, placing operation 1610 may include placing the one or more sample fluids into the one or more separation channels so that the one or more sample fluids are in substantially anti-parallel laminar flow with the one or more second separation fluids.

After a start operation, the operational flow 1600 includes a translocating operation 1620 involving translocating one or more magnetically active constituents from the one or more sample fluids into the one or more first separation fluids. In some embodiments, translocating operation 1620 may include translocating the one or more magnetically active constituents that include one or more non-ferrous tags. In some embodiments, translocating operation 1620 may include translocating the one or more magnetically active constituents that include one or more ferrous tags. In some embodiments, translocating operation 1620 may include translocating the one or more magnetically active constituents that include one or more magnetic tags. In some embodiments, translocating operation 1620 may include translocating the one or more magnetically active constituents that include one or more paramagnetic tags. In some embodiments, translocating operation 1620 may include translocating the one or more magnetically active constituents through use of one or more ferrofluids. In some embodiments, translocating operation 1620 may include translocating the one or more magnetically active constituents through use of one or more magnetically active fluids that include magnetic particles.

After a start operation, the operational flow 1600 includes a translocating operation 1630 involving translocating the one or more magnetically active constituents from the one or more sample fluids into the one or more second separation fluids. In some embodiments, translocating operation 1630 may include translocating the one or more magnetically active constituents that include one or more non-ferrous tags. In some embodiments, translocating operation 1630 may include translocating the one or more magnetically active constituents that include one or more ferrous tags. In some embodiments, translocating operation 1630 may include translocating the one or more magnetically active constituents that include one or more magnetic tags. In some embodiments, translocating operation 1630 may include translocating the one or more magnetically active constituents that include one or more paramagnetic tags. In some embodiments, translocating operation 1630 may include translocating the one or more magnetically active constituents through use of one or more ferrofluids. In some embodiments, translocating operation 1630 may include translocating the one or more magnetically active constituents through use of one or more magnetically active fluids that include magnetic particles.

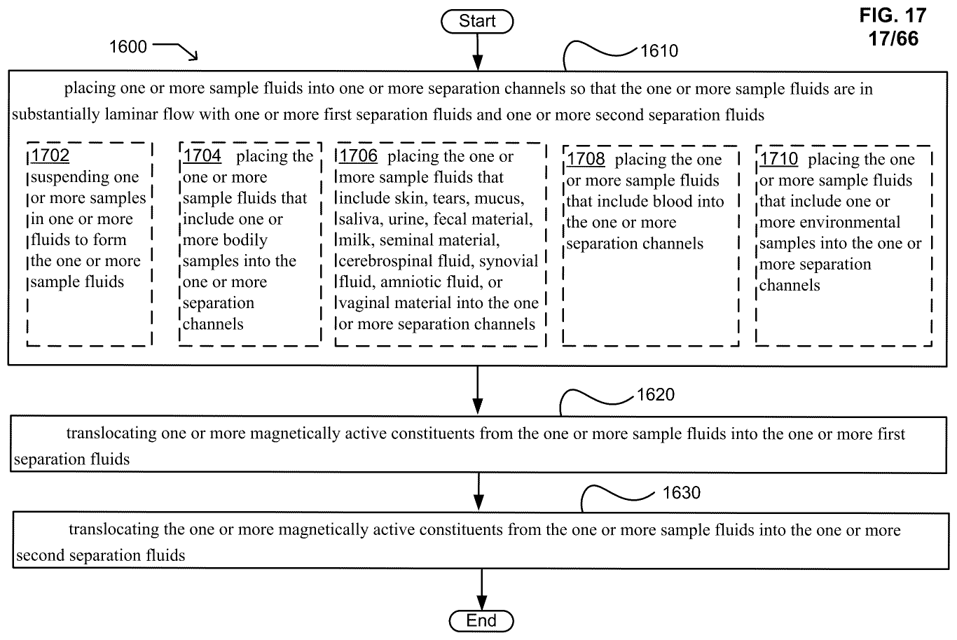

FIG. 17 illustrates alternative embodiments of the example operational flow 1600 of FIG. 16. FIG. 17 illustrates example embodiments where the placing operation 1610 may include at least one additional operation. Additional operations may include an operation 1702, an operation 1704, an operation 1706, an operation 1708, and/or an operation 1710.

At operation 1702, the placing operation 1610 may include suspending one or more samples in one or more fluids to form the one or more sample fluids. In some embodiments, one or more samples 102 may be suspended in one or more fluids to form one or more sample fluids 104. In some embodiments, one or more samples 102 may be dissolved in one or more solvents. Numerous types of samples 102 may be suspended in one or more fluids to form one or more sample fluids 104. Examples of samples 102 include, but are not limited to, solid samples 102, liquid samples 102, semi-solid samples 102, gels, and the like. Such samples 102 may include, but are not limited to, food samples 102, biological samples 102, fuel samples 102, environmental samples 102, crop samples 102, water samples 102, diagnostic samples 102 (e.g., tissue, blood, saliva, mucus, cerebrospinal fluid, amniotic fluid, and the like). In some embodiments, blood samples 102 may be combined with one or more fluids to form one or more sample fluids 104. In some embodiments, one or more fluids may include desired functions. For example, in some embodiments, one or more fluids that include nuclease inhibitors may be mixed with one or more samples 102. In some embodiments, one or more fluids that include one or more RNase inhibitors may be mixed with one or more blood samples 102 to preserve polyribonucleic acids present within the one or more blood samples 102. In some embodiments, precipitates may be suspended in one or more fluids. For example, in some embodiments, precipitated polynucleotides may be suspended in one or more fluids to form one or more sample fluids 104. In some embodiments, one or more fluids may be used to extract one or more components from a first sample. In some embodiments, one or more fluids may be selected to exhibit desired fluid characteristics. Examples of such characteristics include, but are not limited to, viscosity, density, miscibility, solubility, polarity, vapor pressure, flammability, and the like. Accordingly, numerous types of samples 102 and numerous types of fluids may be used to prepare a sample fluid 104.

At operation 1704, the placing operation 1610 may include placing the one or more sample fluids that include one or more bodily samples into the one or more separation channels. In some embodiments, one or more sample fluids 104 that include one or more bodily samples 102 may be placed into one or more separation channels 118. Examples of such bodily samples 102 include, but are not limited to, tissue, tears, saliva, mucus, wax, blood, synovial fluid, cerebrospinal fluid, seminal fluid, vaginal fluid, amniotic fluid, urine, fecal material, and the like. In some embodiments, such samples 102 may be used within diagnostic methods. For example, in some embodiments, amniotic fluid may be used to determine if a fetus exhibits a genotype associated with a disease. In other examples, parasites within fecal material may be detected to determine if an individual is infected with a parasite. Numerous pathogens and parasites have been described (e.g., U.S. patent application Ser. Nos. 11/729,301, 11/729,274, 11/729,276, herein incorporated by reference). Accordingly, numerous types of bodily samples 102 may be selected.

At operation 1706, the placing operation 1610 may include placing the one or more sample fluids that include skin, tears, mucus, saliva, urine, fecal material, milk, seminal material, cerebrospinal fluid, synovial fluid, amniotic fluid, or vaginal material into the one or more separation channels. In some embodiments, one or more sample fluids 104 that include skin, tears, mucus, saliva, urine, fecal material, milk, seminal material, cerebrospinal fluid, synovial fluid, amniotic fluid, vaginal material, or the like may be placed into one or more separation channels 118 in substantially any combination.

At operation 1708, the placing operation 1610 may include placing the one or more sample fluids that include blood into the one or more separation channels. In some embodiments, one or more sample fluids 104 that include blood may be placed into one or more separation channels 118. In some embodiments, one or more blood samples 102 may be collected from one or more blood banks. Accordingly, in some embodiments, blood samples 102 may be screened through use of one or more separation channels 118. In some embodiments, one or more blood samples 102 may be collected from a patient. Accordingly, in some embodiments, blood samples 102 may be used for diagnostic purposes. In some examples, blood samples 102 may be used to detect drug use by an individual.

At operation 1710, the placing operation 1610 may include placing the one or more sample fluids that include one or more environmental samples into the one or more separation channels. In some embodiments, one or more sample fluids 104 that include one or more environmental samples 102 may be placed into one or more separation channels 118. Numerous types of environmental samples 102 may be placed into one or more separation channels 118. Examples of such environmental samples 102 include, but are not limited to, soil samples 102, water samples 102, plant samples 102, farm related samples 102, industrial samples 102, fishery related samples 102, and the like. In some embodiments, environmental samples 102 may include gas (e.g., air) samples 102 that have been filtered through a fluid.

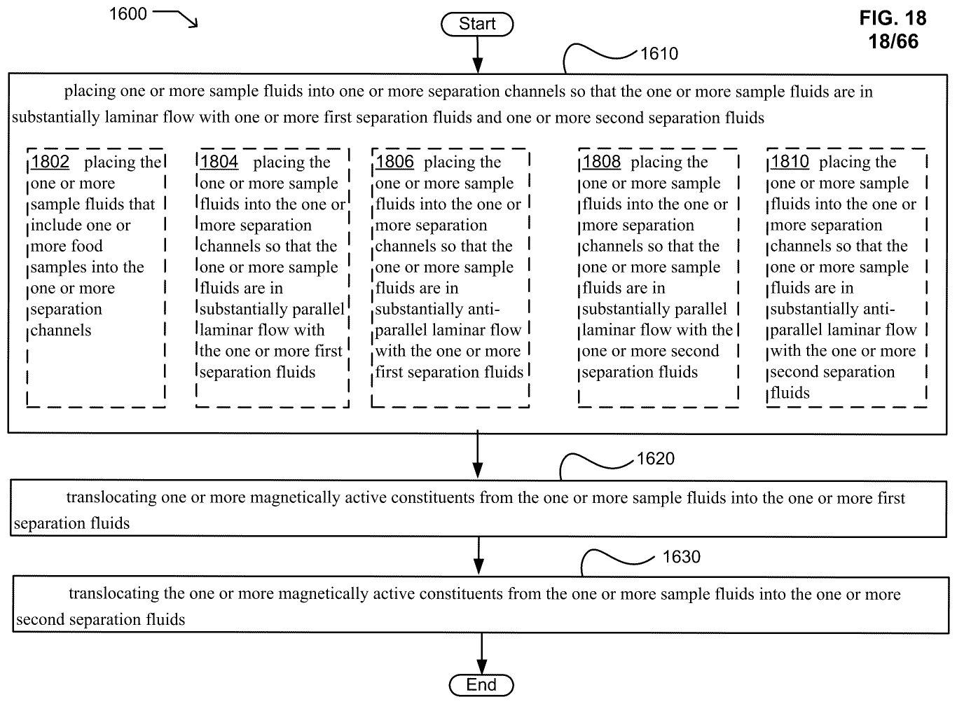

FIG. 18 illustrates alternative embodiments of the example operational flow 1600 of FIG. 16. FIG. 18 illustrates example embodiments where the placing operation 1610 may include at least one additional operation. Additional operations may include an operation 1802, an operation 1804, an operation 1806, an operation 1808, and/or an operation 1810.

At operation 1802, the placing operation 1610 may include placing the one or more sample fluids that include one or more food samples into the one or more separation channels. In some embodiments, one or more sample fluids 104 that include one or more food samples 102 may be placed into one or more separation channels 118. Numerous types of food samples 102 may be placed into one or more separation channels 118. Examples of such food samples 102 include, but are not limited to, vegetables, meats, cheeses, juices, milk, fruits, nuts, prepared foods, raw foods, and the like. Accordingly, components of one or more food samples 102 may be separated through use of one or more separation channels 118. In some embodiments, allergens may be separated from one or more food samples 102 (e.g., U.S. patent application Ser. Nos. 11/699,770, 11/699,920, 11/699,747, 11/699,774, herein incorporated by reference). In some embodiments, pathogens may be separated from one or more food samples 102 (e.g., U.S. patent application Ser. Nos. 11/729,301, 11/729,274, 11/729,276, herein incorporated by reference).

At operation 1804, the placing operation 1610 may include placing the one or more sample fluids into the one or more separation channels so that the one or more sample fluids are in substantially parallel laminar flow with the one or more first separation fluids. In some embodiments, one or more sample fluids 104 may be placed into one or more separation channels 118 so that the one or more sample fluids 104 are in substantially parallel laminar flow with one or more first separation fluids. In such embodiments, the one or more sample fluids 104 flow in the same direction as the one or more first separation fluids. In some embodiments, sample fluids 104 and first separation fluids may be matched to each other. For example, in some embodiments, sample fluids 104 and first separation fluids may be selected that are immiscible with each other. Accordingly, numerous characteristics may be considered when selecting sample fluids 104 and first separation fluids. Examples of such characteristics include, but are not limited to, viscosity, density, miscibility, solubility, vapor pressure, and the like. In some embodiments, the one or more first separation fluids may include a ferrofluid. In some embodiments, the one or more first separation fluids may include paramagnetic particles. In some embodiments, the one or more first separation fluids may include diamagnetic particles. In some embodiments, the one or more first separation fluids may include magnetic particles.

At operation 1806, the placing operation 1610 may include placing the one or more sample fluids into the one or more separation channels so that the one or more sample fluids are in substantially anti-parallel laminar flow with the one or more first separation fluids. In some embodiments, one or more sample fluids 104 may be placed into one or more separation channels 118 so that the one or more sample fluids 104 are in substantially anti-parallel laminar flow with one or more first separation fluids. In such embodiments, the one or more sample fluids 104 flow in the opposite direction as the one or more first separation fluids. In some embodiments, sample fluids 104 and first separation fluids may be matched to each other. For example, in some embodiments, sample fluids 104 and first separation fluids may be selected that are immiscible with each other. Accordingly, numerous characteristics may be considered when selecting sample fluids 104 and first separation fluids. Examples of such characteristics include, but are not limited to, viscosity, density, miscibility, solubility, vapor pressure, and the like. In some embodiments, the one or more first separation fluids may include a ferrofluid. In some embodiments, the one or more first separation fluids may include paramagnetic particles. In some embodiments, the one or more first separation fluids may include diamagnetic particles. In some embodiments, the one or more first separation fluids may include magnetic particles.

At operation 1808, the placing operation 1610 may include placing the one or more sample fluids into the one or more separation channels so that the one or more sample fluids are in substantially parallel laminar flow with the one or more second separation fluids. In some embodiments, one or more sample fluids 104 may be placed into one or more separation channels 118 so that the one or more sample fluids 104 are in substantially parallel laminar flow with one or more second separation fluids. In such embodiments, the one or more sample fluids 104 flow in the same direction as the one or more second separation fluids. In some embodiments, sample fluids 104 and second separation fluids may be matched to each other. For example, in some embodiments, sample fluids 104 and second separation fluids may be selected that are immiscible with each other. Accordingly, numerous characteristics may be considered when selecting sample fluids 104 and second separation fluids. Examples of such characteristics include, but are not limited to, viscosity, density, miscibility, solubility, vapor pressure, and the like. In some embodiments, the one or more second separation fluids may include a ferrofluid. In some embodiments, the one or more second separation fluids may include paramagnetic particles. In some embodiments, the one or more second separation fluids may include diamagnetic particles. In some embodiments, the one or more second separation fluids may include magnetic particles.

At operation 1810, the placing operation 1610 may include placing the one or more sample fluids into the one or more separation channels so that the one or more sample fluids are in substantially anti-parallel laminar flow with the one or more second separation fluids. In some embodiments, one or more sample fluids 104 may be placed into one or more separation channels 118 so that the one or more sample fluids 104 are in substantially anti-parallel laminar flow with one or more second separation fluids. In such embodiments, the one or more sample fluids 104 flow in the opposite direction as the one or more second separation fluids. In some embodiments, sample fluids 104 and second separation fluids may be matched to each other. For example, in some embodiments, sample fluids 104 and second separation fluids may be selected that are immiscible with each other. Accordingly, numerous characteristics may be considered when selecting sample fluids 104 and second separation fluids. Examples of such characteristics include, but are not limited to, viscosity, density, miscibility, solubility, vapor pressure, and the like. In some embodiments, the one or more second separation fluids may include a ferrofluid. In some embodiments, the one or more second separation fluids may include paramagnetic particles. In some embodiments, the one or more second separation fluids may include diamagnetic particles. In some embodiments, the one or more second separation fluids may include magnetic particles.

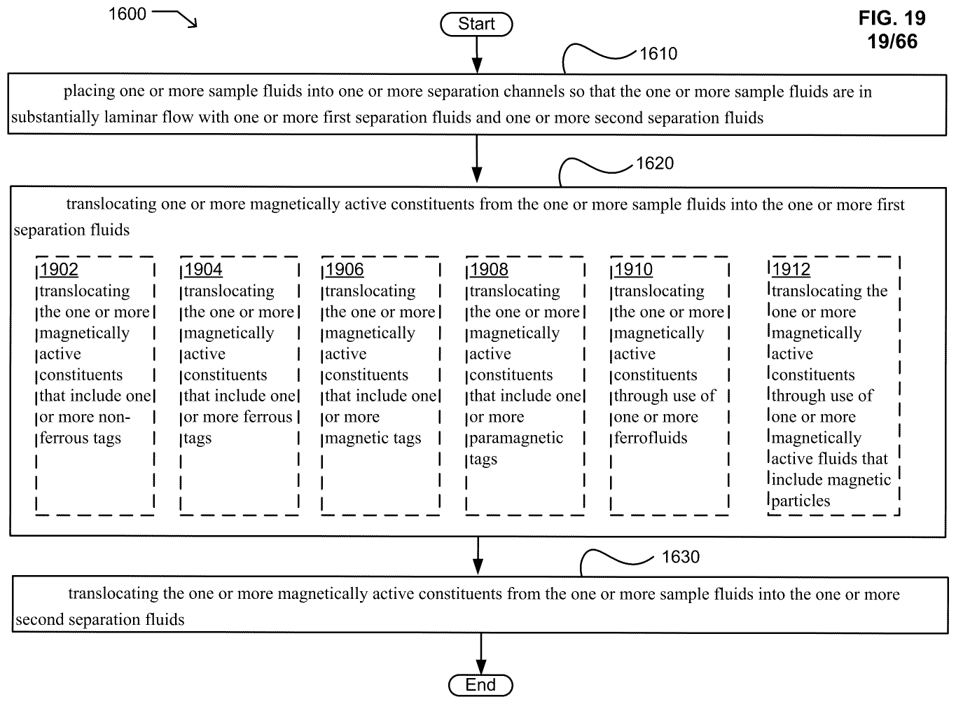

FIG. 19 illustrates alternative embodiments of the example operational flow 1600 of FIG. 16. FIG. 19 illustrates example embodiments where the translocating operation 1620 may include at least one additional operation. Additional operations may include an operation 1902, an operation 1904, an operation 1906, an operation 1908, an operation 1910, and/or an operation 1912.

At operation 1902, the translocating operation 1620 may include translocating the one or more magnetically active constituents that include one or more non-ferrous tags. In some embodiments, one or more magnetically active constituents 106 that include one or more non-ferrous tags may be translocated. Numerous types of non-ferrous tags may be transported. In some embodiments, non-ferrous tags may be magnetic. Examples of non-ferrous permanent magnets 124 include, but are not limited to, alnico magnets 124, samarium-cobalt magnets 124, plastic magnets 124, and the like. In some embodiments, non-ferrous tags may be paramagnetic. In some embodiments, non-ferrous tags may be diamagnetic. In some embodiments, non-ferrous tags and magnetically active fluids 126 may be matched to each other. For example, in some embodiments, a non-ferrous tag that is a permanent magnet 124 may be matched with a magnetically active fluid 126 to which the tag is attracted to facilitate translocation of the tag. In some embodiments, a non-ferrous tag that is paramagnetic may be matched with a magnetically active fluid 126 to which the tag is attracted to facilitate translocation of the tag. In some embodiments, a non-ferrous tag may be selected that is repelled by a permanent magnet 124.

At operation 1904, the translocating operation 1620 may include translocating the one or more magnetically active constituents that include one or more ferrous tags. In some embodiments, one or more magnetically active constituents 106 that include one or more ferrous tags may be translocated. Numerous types of ferrous tags may be transported. In some embodiments, ferrous tags may be magnetic. Examples of ferrous permanent magnets 124 include, but are not limited to, neodymium magnets 124, ceramic magnets 124, ferromagnets 124, and the like. In some embodiments, ferrous tags and magnetically active fluids 126 may be matched to each other. For example, in some embodiments, a ferrous tag that is a permanent magnet 124 may be matched with a magnetically active fluid 126 to which the tag is attracted to facilitate translocation of the tag. In some embodiments, a ferrous tag that is paramagnetic may be matched with a magnetically active fluid 126 to which the tag is attracted to facilitate translocation of the tag.

At operation 1906, the translocating operation 1620 may include translocating the one or more magnetically active constituents that include one or more magnetic tags. In some embodiments, one or more magnetically active constituents 106 that include one or more magnetic tags may be translocated. Numerous types of magnetic tags may be transported. Examples of magnetic tags include, but are not limited to, neodymium magnets, ceramic magnets, ferromagnets, alnico magnets, samarium-cobalt magnets, plastic magnets, and the like. In some embodiments, magnetic tags and magnetically active fluids 126 may be matched to each other. For example, in some embodiments, a magnetic tag that is a permanent magnet 124 may be matched with a magnetically active fluid 126 to which the tag is attracted to facilitate translocation of the tag.

At operation 1908, the translocating operation 1620 may include translocating the one or more magnetically active constituents that include one or more paramagnetic tags. In some embodiments, one or more magnetically active constituents 106 that include one or more paramagnetic tags may be translocated. Numerous types of paramagnetic tags may be transported. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like. In some embodiments, paramagnetic tags and magnetically active fluids 126 may be matched to each other. For example, in some embodiments, a paramagnetic tag may be matched with a magnetically active fluid 126 to which the tag is attracted to facilitate translocation of the tag. In some embodiments, one or more magnets 124 may be used to facilitate translocation of one or more magnetically active constituents 106.

At operation 1910, the translocating operation 1620 may include translocating the one or more magnetically active constituents through use of one or more ferrofluids. In some embodiments, one or more magnetically active constituents 106 may be translocated through use of one or more ferrofluids. In some embodiments, a magnetically active constituent 106 may include a magnet 124 that is attracted to one or more ferrofluids and thereby facilitates translocation of the magnetically active constitutent 106 into the ferrofluid. Numerous types of ferrofluids may be utilized. For example, in some embodiments, one or more ferrofluids may be used that are suitable biological buffers. Accordingly, the activity and/or integrity of biological materials may be preserved following translocation into such ferrofluids. In some embodiments, ferrofluids may be selected that are matched to one or more sample fluids 104, one or more second separation fluids, or substantially any combination thereof. Ferrofluids may be selected that exhibit numerous characteristics that include, but are not limited to, viscosity, density, miscibility, solvent characteristics, vapor pressure, freezing temperature, and the like.

At operation 1912, the translocating operation 1620 may include translocating the one or more magnetically active constituents through use of one or more magnetically active fluids that include magnetic particles. In some embodiments, one or more magnetically active constituents 106 may be translocated through use of one or more magnetically active fluids 126 that include magnetic particles. In some embodiments, the magnetic particles may be coated with one or more surfactants. Examples of such surfactants include, but are not limited to, oleic acid, tetramethylammonium hydroxide, citric acid, soy lecithin, and the like. Magnetic particles may be prepared from numerous materials that are known and have been described.

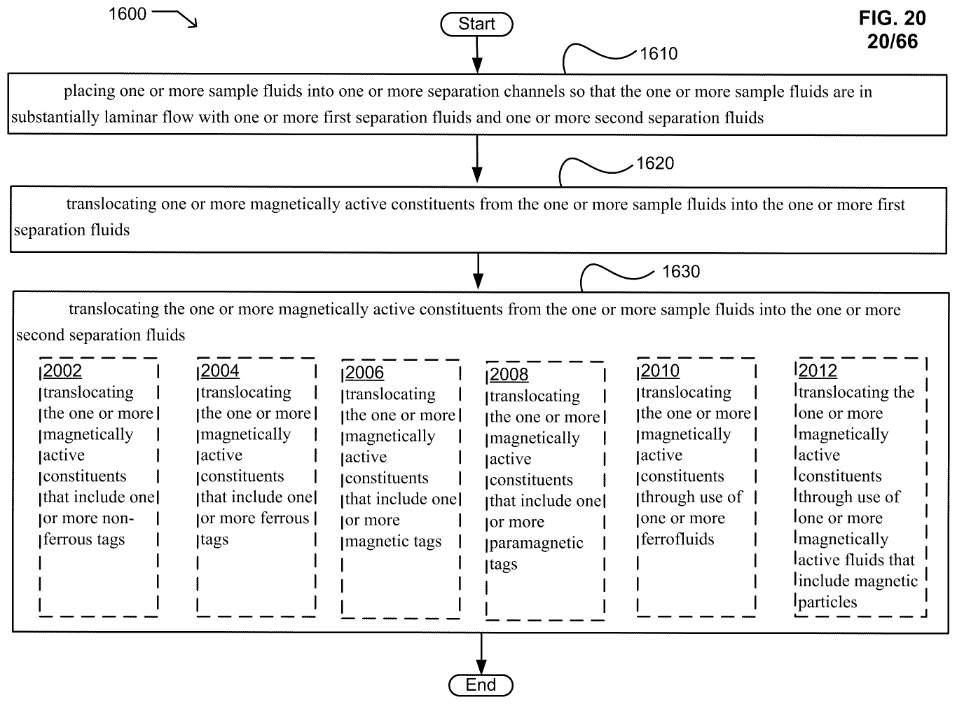

FIG. 20 illustrates alternative embodiments of the example operational flow 1600 of FIG. 16. FIG. 20 illustrates example embodiments where the translocating operation 1630 may include at least one additional operation. Additional operations may include an operation 2002, an operation 2004, an operation 2006, an operation 2008, an operation 2010, and/or an operation 2012.

At operation 2002, the translocating operation 1630 may include translocating the one or more magnetically active constituents that include one or more non-ferrous tags. In some embodiments, one or more magnetically active constituents 106 that include one or more non-ferrous tags may be translocated. Numerous types of non-ferrous tags may be transported. In some embodiments, non-ferrous tags may be magnetic. Examples of non-ferrous permanent magnets 124 include, but are not limited to, alnico magnets 124, samarium-cobalt magnets 124, plastic magnets 124, and the like. In some embodiments, non-ferrous tags may be paramagnetic. In some embodiments, non-ferrous tags may be diamagnetic. In some embodiments, non-ferrous tags and magnetically active fluids 126 may be matched to each other. For example, in some embodiments, a non-ferrous tag that is a permanent magnet 124 may be matched with a magnetically active fluid 126 to which the tag is attracted to facilitate translocation of the tag. In some embodiments, a non-ferrous tag that is paramagnetic may be matched with a magnetically active fluid 126 to which the tag is attracted to facilitate translocation of the tag. In some embodiments, a non-ferrous tag may be selected that is repelled by a permanent magnet 124.

At operation 2004, the translocating operation 1630 may include translocating the one or more magnetically active constituents that include one or more ferrous tags. In some embodiments, one or more magnetically active constituents 106 that include one or more ferrous tags may be translocated. Numerous types of ferrous tags may be transported. In some embodiments, ferrous tags may be magnetic. Examples of ferrous permanent magnets 124 include, but are not limited to, neodymium magnets 124, ceramic magnets 124, ferromagnets 124, and the like. In some embodiments, ferrous tags and magnetically active fluids 126 may be matched to each other. For example, in some embodiments, a ferrous tag that is a permanent magnet 124 may be matched with a magnetically active fluid 126 to which the tag is attracted to facilitate translocation of the tag. In some embodiments, a ferrous tag that is paramagnetic may be matched with a magnetically active fluid 126 to which the tag is attracted to facilitate translocation of the tag.

At operation 2006, the translocating operation 1630 may include translocating the one or more magnetically active constituents that include one or more magnetic tags. In some embodiments, one or more magnetically active constituents 106 that include one or more magnetic tags may be translocated. Numerous types of magnetic tags may be transported. Examples of magnetic tags include, but are not limited to, neodymium magnets, ceramic magnets, ferromagnets, alnico magnets, samarium-cobalt magnets, plastic magnets, and the like. In some embodiments, magnetic tags and magnetically active fluids 126 may be matched to each other. For example, in some embodiments, a magnetic tag that is a permanent magnet 124 may be matched with a magnetically active fluid 126 to which the tag is attracted to facilitate translocation of the tag.

At operation 2008, the translocating operation 1630 may include translocating the one or more magnetically active constituents that include one or more paramagnetic tags. In some embodiments, one or more magnetically active constituents 106 that include one or more paramagnetic tags may be translocated. Numerous types of paramagnetic tags may be transported. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like. In some embodiments, paramagnetic tags and magnetically active fluids 126 may be matched to each other. For example, in some embodiments, a paramagnetic tag may be matched with a magnetically active fluid 126 to which the tag is attracted to facilitate translocation of the tag. In some embodiments, one or more magnets 124 may be used to facilitate translocation of one or more magnetically active constituents 106.

At operation 2010, the translocating operation 1630 may include translocating the one or more magnetically active constituents through use of one or more ferrofluids. In some embodiments, one or more magnetically active constituents 106 may be translocated through use of one or more ferrofluids. In some embodiments, a magnetically active constituent 106 may include a magnet 124 that is attracted to one or more ferrofluids and thereby facilitates translocation of the magnetically active constitutent 106 into the ferrofluid. Numerous types of ferrofluids may be utilized. For example, in some embodiments, one or more ferrofluids may be used that are suitable biological buffers. Accordingly, the activity and/or integrity of biological materials may be preserved following translocation into such ferrofluids. In some embodiments, ferrofluids may be selected that are matched to one or more sample fluids 104, one or more second separation fluids, or substantially any combination thereof. Ferrofluids may be selected that exhibit numerous characteristics that include, but are not limited to, viscosity, density, miscibility, solvent characteristics, vapor pressure, freezing temperature, and the like.

At operation 2012, the translocating operation 1630 may include translocating the one or more magnetically active constituents through use of one or more magnetically active fluids that include magnetic particles. In some embodiments, one or more magnetically active constituents 106 may be translocated through use of one or more magnetically active fluids 126 that include magnetic particles. In some embodiments, the magnetic particles may be coated with one or more surfactants. Examples of such surfactants include, but are not limited to, oleic acid, tetramethylammonium hydroxide, citric acid, soy lecithin, and the like. Magnetic particles may be prepared from numerous materials that are known and have been described.

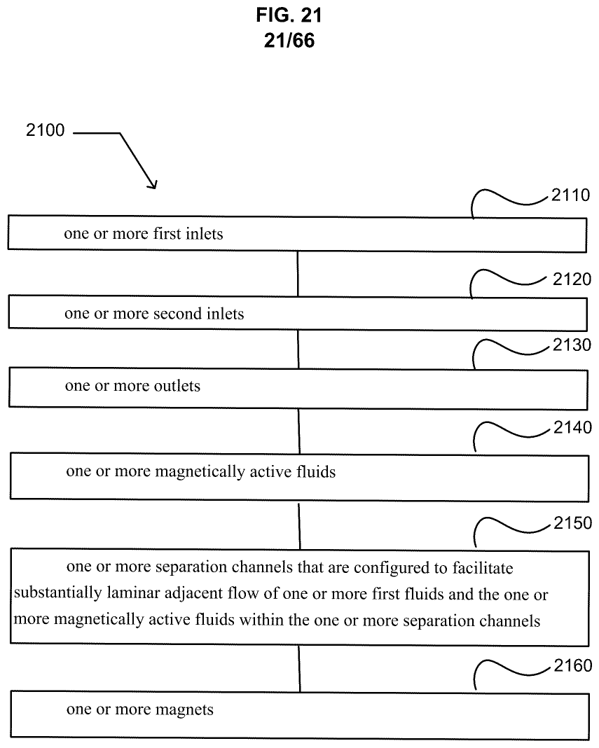

FIG. 21 illustrates a device 2100 representing examples of modules that may be used to perform a method for separating one or more constituents from one or more samples 102. In FIG. 21, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The device 2100 includes module 2110 that includes one or more first inlets. In some embodiments, module 2110 may include one or more first fluid inlets. In some embodiments, module 2110 may include one or more sample fluid inlets.

The device 2100 includes module 2120 that includes one or more second inlets. In some embodiments, module 2120 may include one or more magnetically active fluid inlets.

The device 2100 includes module 2130 that includes one or more outlets. In some embodiments, module 2130 may include one or more first fluid outlets. In some embodiments, module 2130 may include one or more sample fluid outlets. In some embodiments, module 2130 may include one or more magnetically active fluid outlets. In some embodiments, module 2130 may include one or more first fluid outlets and one or more magnetically active fluid outlets. In some embodiments, module 2130 may include one or more detection chambers.

The device 2100 includes module 2140 that includes one or more magnetically active fluids. In some embodiments, module 2140 may include one or more magnetically active extraction fluids that include magnetic particles. In some embodiments, module 2140 may include one or more magnetically active fluids that include paramagnetic particles.

The device 2100 includes module 2150 that includes one or more separation channels that are configured to facilitate substantially laminar adjacent flow of one or more first fluids and the one or more magnetically active fluids within the one or more separation channels. In some embodiments, module 2150 may include one or more separation channels that are configured to facilitate substantially parallel laminar adjacent flow. In some embodiments, module 2150 may include one or more separation channels that are configured to facilitate substantially anti-parallel laminar adjacent flow. In some embodiments, module 2150 may include one or more detection chambers.

The device 2100 may optionally include module 2160 that includes one or more magnets. In some embodiments, module 2160 may include one or more magnets that are configured to facilitate translocation of one or more magnetically active components from the one or more first fluids to the one or more magnetically active fluids. In some embodiments, module 2160 may include one or more attractive magnets. In some embodiments, module 2160 may include one or more repulsive magnets.

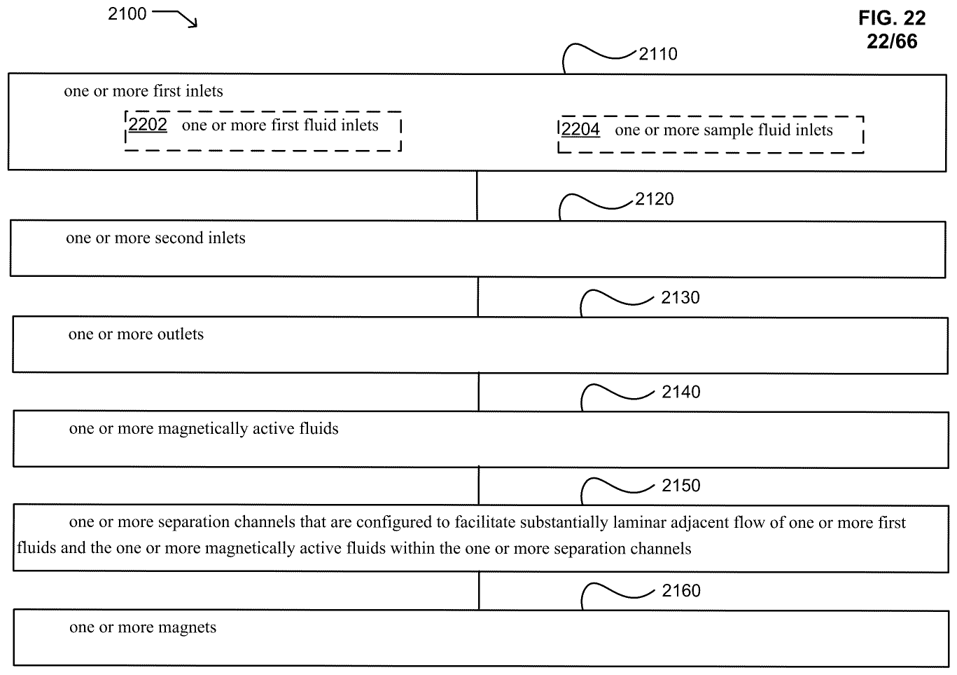

FIG. 22 illustrates alternative embodiments of device 2100 of FIG. 21. FIG. 22 illustrates example embodiments of module 2110. Additional embodiments may include an embodiment 2202, and/or an embodiment 2204.

At embodiment 2202, module 2110 may include one or more first fluid inlets.

In some embodiments, a device may include one or more first fluid inlets 112 that are operably associated with one or more separation channels 118. Fluid inlets may be configured in numerous ways. For example, in some embodiments, a fluid inlet may be configured to include one or more septa through which one or more first fluids 120 may be injected through use of a syringe. In some embodiments, a fluid inlet may be configured to include one or more fittings (e.g., leur lock fittings) through which one or more first fluids 120 may be injected through use of a syringe. In some embodiments, a fluid inlet may be configured to include one or more fittings to which one or more pumps may be attached to pump one or more sample fluids 104 into the device.

At embodiment 2204, module 2110 may include one or more sample fluid inlets. In some embodiments, a device may include one or more sample fluid inlets that are operably associated with one or more separation channels 118. Sample fluid inlets may be configured in numerous ways. For example, in some embodiments, a sample fluid inlet may be configured to include one or more septa through which one or more sample fluids 104 may be injected through use of a syringe. In some embodiments, a sample fluid 104 inlet may be configured to include one or more fittings (e.g., leur lock fittings) through which one or more sample fluids 104 may be injected through use of a syringe. In some embodiments, a sample fluid inlet may be configured to include one or more fittings to which one or more pumps may be attached to pump one or more sample fluids 104 into the device.

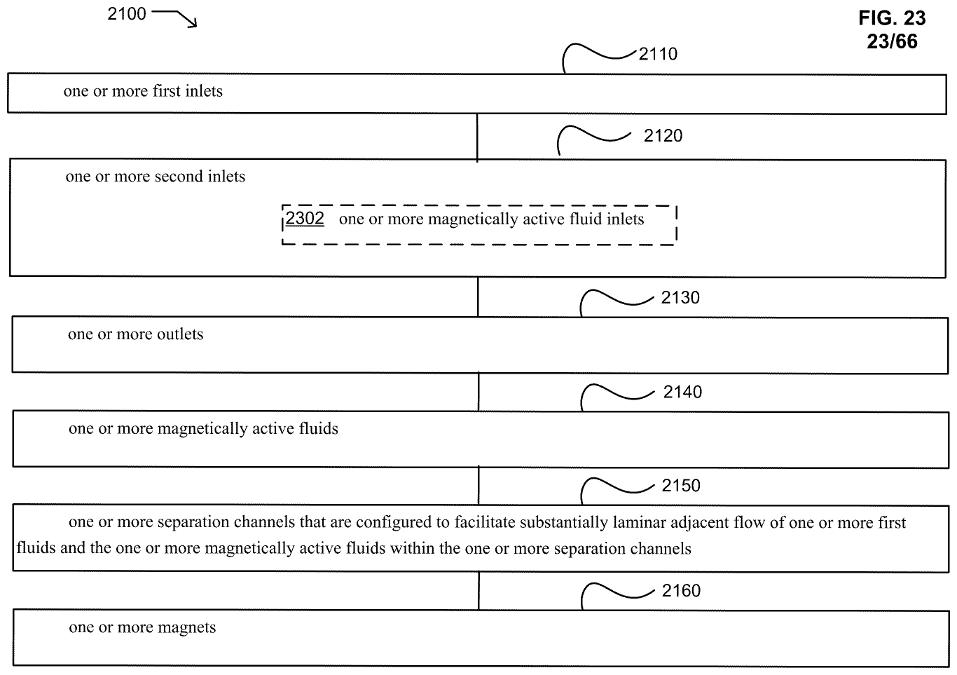

FIG. 23 illustrates alternative embodiments of device 2100 of FIG. 21. FIG. 23 illustrates example embodiments of module 2120. Additional embodiments may include an embodiment 2302.

At embodiment 2302, module 2120 may include one or more magnetically active fluid inlets. In some embodiments, a device may include one or more magnetically active fluid inlets that are operably associated with one or more separation channels 118. Magnetically active fluid inlets may be configured in numerous ways. For example, in some embodiments, a magnetically active fluid inlet may be configured to include one or more septa through which one or more magnetically active fluids 126 may be injected through use of a syringe. In some embodiments, a magnetically active fluid inlet may be configured to include one or more fittings (e.g., leur lock fittings) through which one or more magnetically active fluids 126 may be injected through use of a syringe. In some embodiments, a magnetically active fluid inlet may be configured to include one or more fittings to which one or more pumps may be attached to pump one or more magnetically active fluids 126 into the device.

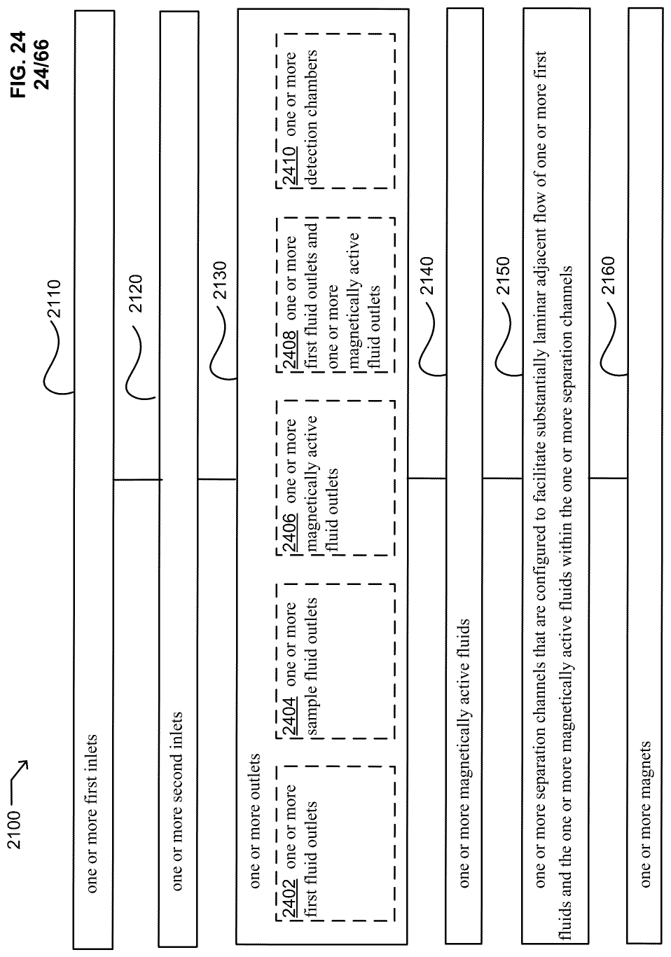

FIG. 24 illustrates alternative embodiments of device 2100 of FIG. 21. FIG. 24 illustrates example embodiments of module 2130. Additional embodiments may include an embodiment 2402, an embodiment 2404, an embodiment 2406, an embodiment 2408, and/or an embodiment 2410.

At embodiment 2402, module 2130 may include one or more first fluid outlets. In some embodiments, a device may include one or more first fluid outlets 116 that are operably associated with one or more separation channels 118. In some embodiments, a device may include one or more first fluid outlets 116 that are operably associated with one or more substantially continuous fluid channels 128.

At embodiment 2404, module 2130 may include one or more sample fluid outlets. In some embodiments, a device may include one or more sample fluid outlets that are operably associated with one or more separation channels 118. In some embodiments, a device may include one or more sample fluid outlets that are operably associated with one or more substantially continuous fluid channels 128. In some embodiments, a device may include one or more sample fluid outlets that are operably associated with one or more additional separation channels 118.

At embodiment 2406, module 2130 may include one or more magnetically active fluid outlets. In some embodiments, a device may include one or more magnetically active fluid outlets 116 that are operably associated with one or more separation channels 118. In some embodiments, a device may include one or more magnetically active fluid outlets 116 that are operably associated with one or more substantially continuous fluid channels 128.

At embodiment 2408, module 2130 may include one or more first fluid outlets and one or more magnetically active fluid outlets. In some embodiments, a device may include one or more first fluid outlets 116 and one or more magnetically active fluid outlets 116 that are operably associated with one or more separation channels 118. In some embodiments, a device may include one or more magnetically active fluid outlets 116 that are operably associated with one or more substantially continuous fluid channels 128. In some embodiments, such devices may be configured for continuous separation of components from one or more sample fluids 104.

At embodiment 2410, module 2130 may include one or more detection chambers.

In some embodiments, a device may include one or more detection chambers that are operably associated with one or more separation channels 118. Detection chambers may be configured to detect one or more components through use of numerous technologies. Examples of such technologies include, but are not limited to, spectroscopy, electrochemical detection, polynucleotide detection, fluorescence anisotropy, fluorescence resonance energy transfer, electron transfer, enzyme assay, electrical conductivity, isoelectric focusing, chromatography, immunoprecipitation, immunoseparation, aptamer binding, electrophoresis, use of a CCD camera, immunoassay, polypeptide detection, antibody interaction, chemical interaction, diffusion, filtration, aptamer interaction, magnetism, competition assay, or substantially any combination thereof.

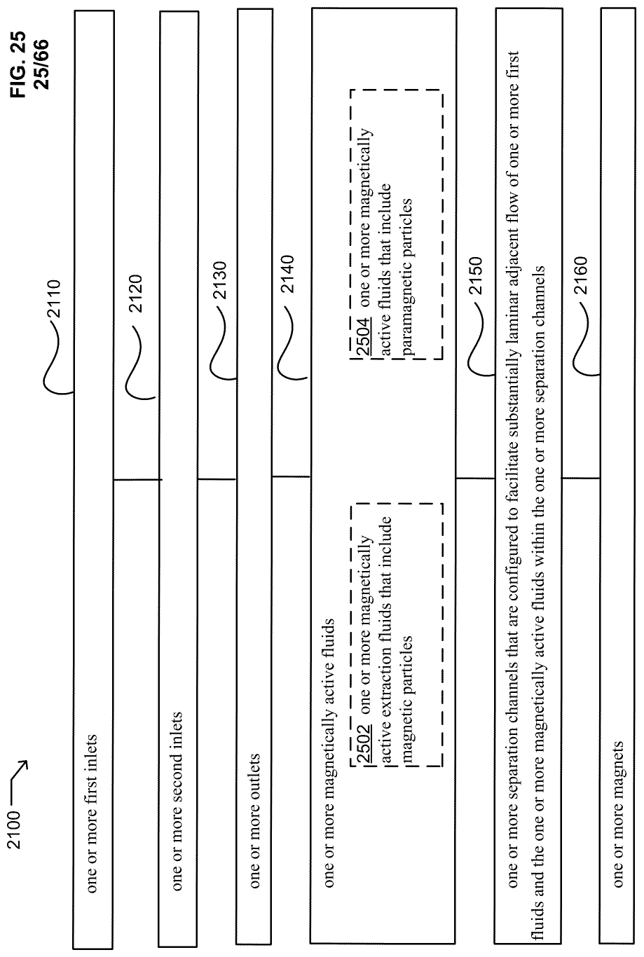

FIG. 25 illustrates alternative embodiments of device 2100 of FIG. 21. FIG. 25 illustrates example embodiments of module 2140. Additional embodiments may include an embodiment 2502, and/or an embodiment 2504.

At embodiment 2502, module 2140 may include one or more magnetically active extraction fluids that include magnetic particles. In some embodiments, one or more devices may include one or more magnetically active extraction fluids that include magnetic particles. In some embodiments, the one or more magnetically active extraction fluids may include a ferrofluid. In some embodiments, the one or more magnetically active extraction fluids may include paramagnetic particles. In some embodiments, the one or more magnetically active extraction fluids may include diamagnetic particles. In some embodiments, the magnetic particles may be coated with a surfactant. Examples of such surfactants include, but are not limited to, oleic acid, tetramethylammonium hydroxide, citric acid, soy lecithin, and the like.

At embodiment 2504, module 2140 may include one or more magnetically active fluids that include paramagnetic particles. In some embodiments, one or more devices may include one or more magnetically active fluids 126 that include paramagnetic particles. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like.

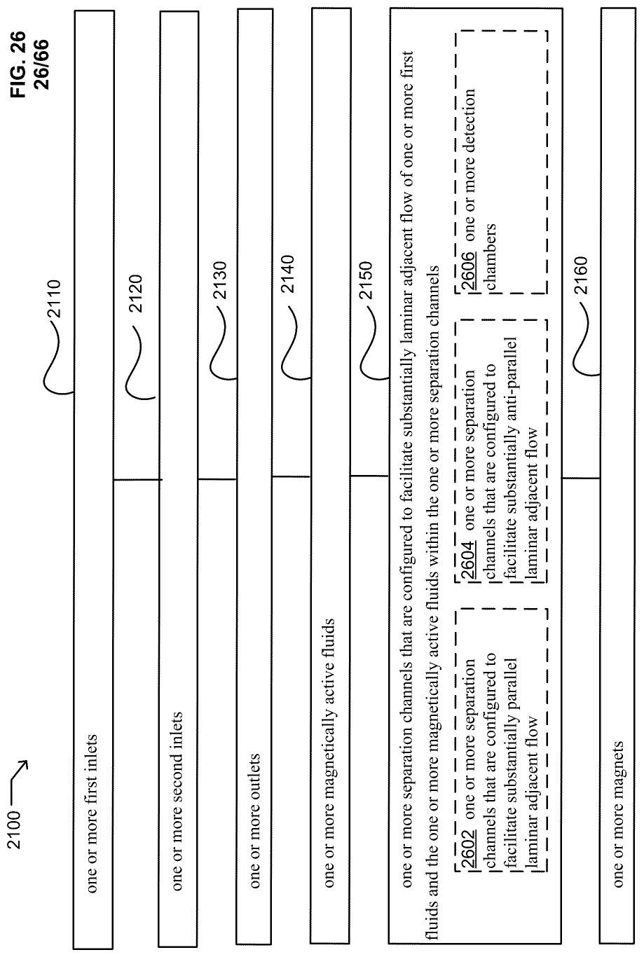

FIG. 26 illustrates alternative embodiments of device 2100 of FIG. 21. FIG. 26 illustrates example embodiments of module 2150. Additional embodiments may include an embodiment 2602, an embodiment 2604, and/or an embodiment 2606.

At embodiment 2602, module 2150 may include one or more separation channels that are configured to facilitate substantially parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially the same direction.

At embodiment 2604, module 2150 may include one or more separation channels that are configured to facilitate substantially anti-parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially opposite directions.

At embodiment 2606, module 2150 may include one or more detection chambers. In some embodiments, a device may include one or more detection chambers that are operably associated with one or more separation channels 118. Detection chambers may be configured to detect one or more components through use of numerous technologies. Examples of such technologies include, but are not limited to, spectroscopy, electrochemical detection, polynucleotide detection, fluorescence anisotropy, fluorescence resonance energy transfer, electron transfer, enzyme assay, electrical conductivity, isoelectric focusing, chromatography, immunoprecipitation, immunoseparation, aptamer binding, electrophoresis, use of a CCD camera, immunoassay, polypeptide detection, antibody interaction, chemical interaction, diffusion, filtration, chromatography, aptamer interaction, magnetism, competition assay, or substantially any combination thereof.

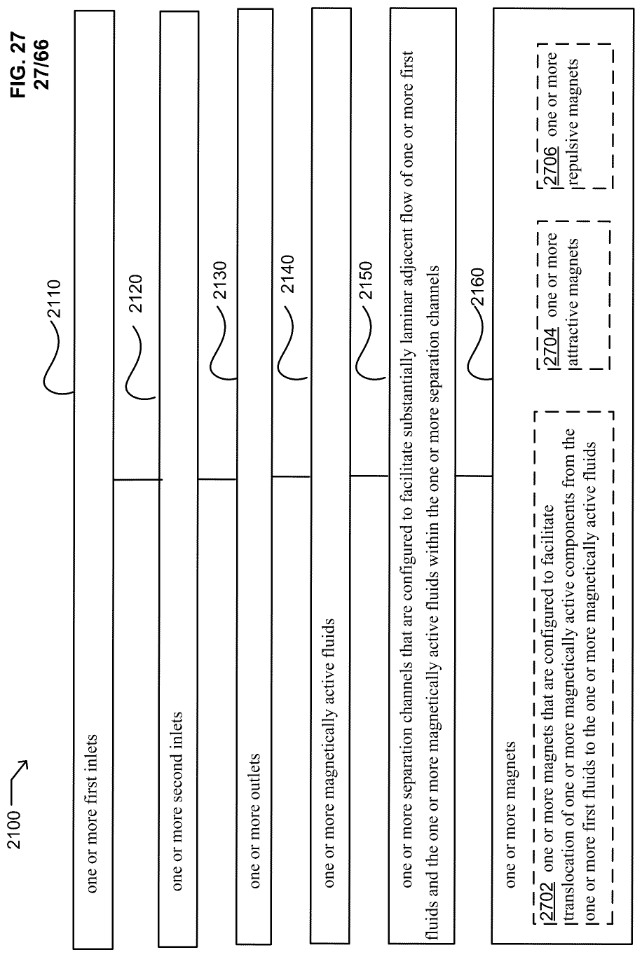

FIG. 27 illustrates alternative embodiments of device 2100 of FIG. 21. FIG. 27 illustrates example embodiments of module 2160. Additional embodiments may include an embodiment 2702, an embodiment 2704, and/or an embodiment 2706.

At embodiment 2702, module 2160 may include one or more magnets that are configured to facilitate translocation of one or more magnetically active components from the one or more first fluids to the one or more magnetically active fluids. In some embodiments, a device may include one or more magnets 124 that are configured to facilitate translocation of one or more magnetically active components from one or more first fluids 120 to one or more magnetically active fluids. In some embodiments, the one or more magnets 124 may be configured to utilize magnetic attraction. In some embodiments, the one or more magnets 124 may be configured to utilize magnetic repulsion.

At embodiment 2704, module 2160 may include one or more attractive magnets. In some embodiments, a device may include one or more attractive magnets 124. In some embodiments, the one or more attractive magnets 124 may include one or more permanent magnets 124. In some embodiments, the one or more attractive magnets 124 may include one or more electromagnets 124. In some embodiments, the one or more attractive magnets 124 facilitate translocation of one or more magnetically active constituents 106 through magnetic attraction.

At embodiment 2706, module 2160 may include one or more repulsive magnets. In some embodiments, a device may include one or more repulsive magnets. In some embodiments, the one or more repulsive magnets 124 may include one or more permanent magnets. In some embodiments, the one or more repulsive magnets 124 may include one or more electromagnets. In some embodiments, the one or more repulsive magnets 124 facilitate translocation of one or more magnetically active constituents 106 through magnetic repulsion.

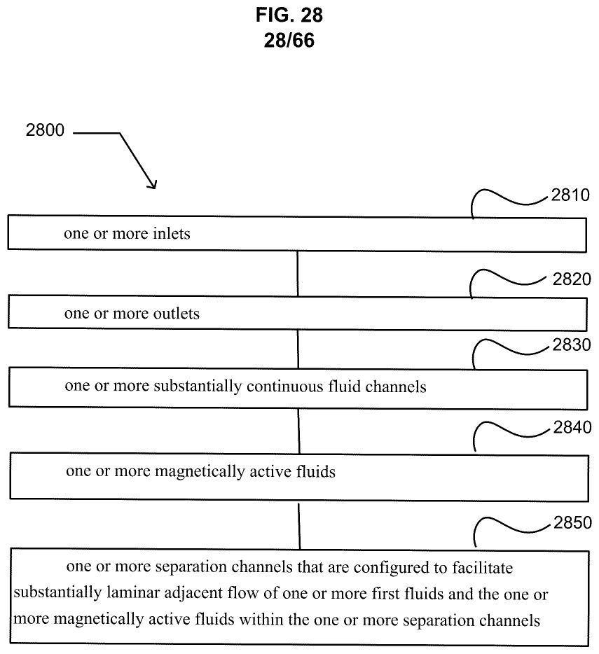

FIG. 28 illustrates a device 2800 representing examples of modules that may be used to perform a method for separating one or more constituents from one or more samples 102. In FIG. 28, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The device 2800 includes module 2810 that includes one or more inlets. In some embodiments, module 2810 may include one or more first fluid inlets. In some embodiments, module 2810 may include one or more sample fluid inlets.

The device 2800 includes module 2820 that includes one or more outlets. In some embodiments, module 2820 may include one or more first fluid outlets. In some embodiments, module 2820 may include one or more sample fluid outlets.

The device 2800 includes module 2830 that includes one or more substantially continuous fluid channels. In some embodiments, module 2830 may include the one or more magnetically active fluids. In some embodiments, module 2830 may include one or more detection chambers.

The device 2800 includes module 2840 that includes one or more magnetically active fluids. In some embodiments, module 2840 may include one or more magnetically active fluids that include magnetic particles. In some embodiments, module 2840 may include one or more magnetically active fluids that include paramagnetic particles. In some embodiments, module 2840 may include one or more ferrofluids.

The device 2800 includes module 2850 that includes one or more separation channels that are configured to facilitate substantially laminar adjacent flow of one or more first fluids and the one or more magnetically active fluids within the one or more separation channels. In some embodiments, module 2850 may include one or more separation channels that are configured to facilitate substantially parallel laminar adjacent flow. In some embodiments, module 2850 may include one or more separation channels that are configured to facilitate substantially anti-parallel laminar adjacent flow.

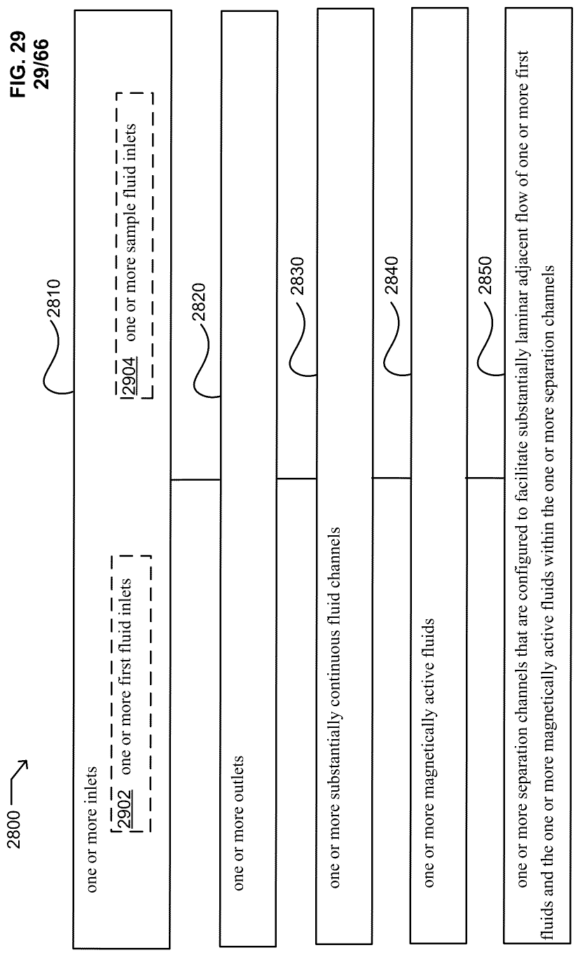

FIG. 29 illustrates alternative embodiments of device 2800 of FIG. 28. FIG. 29 illustrates example embodiments of module 2810. Additional embodiments may include an embodiment 2902, and/or an embodiment 2904.

At embodiment 2902, module 2810 may include one or more first fluid inlets. In some embodiments, a device may include one or more first fluid inlets 112 that are operably associated with one or more separation channels 118. Fluid inlets may be configured in numerous ways. For example, in some embodiments, a fluid inlet may be configured to include one or more septa through which one or more first fluids 120 may be injected through use of a syringe. In some embodiments, a fluid inlet may be configured to include one or more fittings (e.g., leur lock fittings) through which one or more first fluids 120 may be injected through use of a syringe. In some embodiments, a fluid inlet may be configured to include one or more fittings to which one or more pumps may be attached to pump one or more sample fluids 104 into the device.

At embodiment 2904, module 2810 may include one or more sample fluid inlets. In some embodiments, a device may include one or more sample fluid inlets that are operably associated with one or more separation channels 118. Sample fluid inlets may be configured in numerous ways. For example, in some embodiments, a sample fluid inlet may be configured to include one or more septa through which one or more sample fluids 104 may be injected through use of a syringe. In some embodiments, a sample fluid inlet may be configured to include one or more fittings (e.g., leur lock fittings) through which one or more sample fluids 104 may be injected through use of a syringe. In some embodiments, a sample fluid inlet may be configured to include one or more fitting to which one or more pumps may be attached to pump one or more sample fluids 104 into the device.

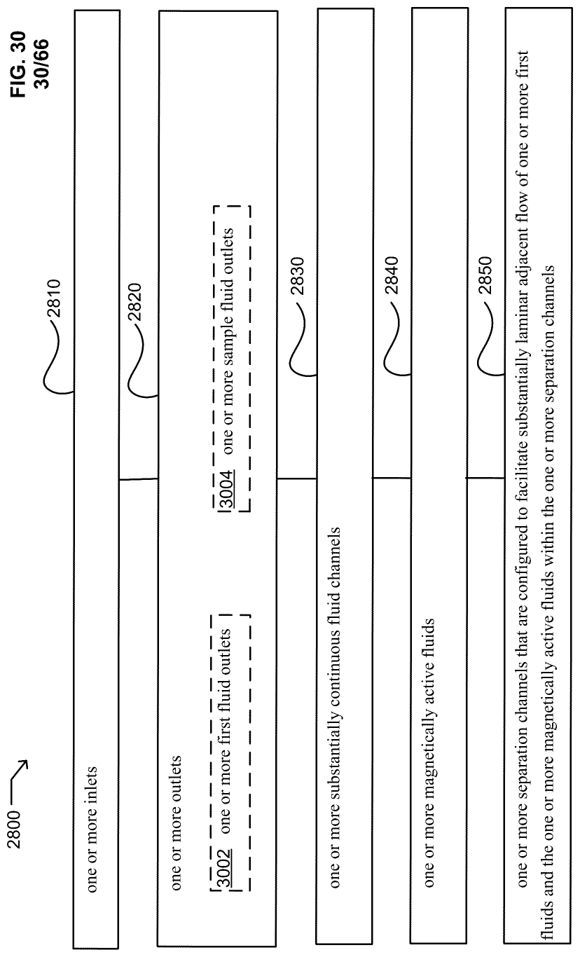

FIG. 30 illustrates alternative embodiments of device 2800 of FIG. 28. FIG. 30 illustrates example embodiments of module 2820. Additional embodiments may include an embodiment 3002, and/or an embodiment 3004.

At embodiment 3002, module 2820 may include one or more first fluid outlets. In some embodiments, a device may include one or more first fluid outlets 116 that are operably associated with one or more separation channels 118. In some embodiments, a device may include one or more first fluid outlets 116 that are operably associated with one or more substantially continuous fluid channels 128.

At embodiment 3004, module 2820 may include one or more sample fluid outlets. In some embodiments, a device may include one or more sample fluid outlets that are operably associated with one or more separation channels 118. In some embodiments, a device may include one or more sample fluid outlets that are operably associated with one or more substantially continuous fluid channels 128. In some embodiments, a device may include one or more sample fluid outlets that are operably associated with one or more additional separation channels 118.

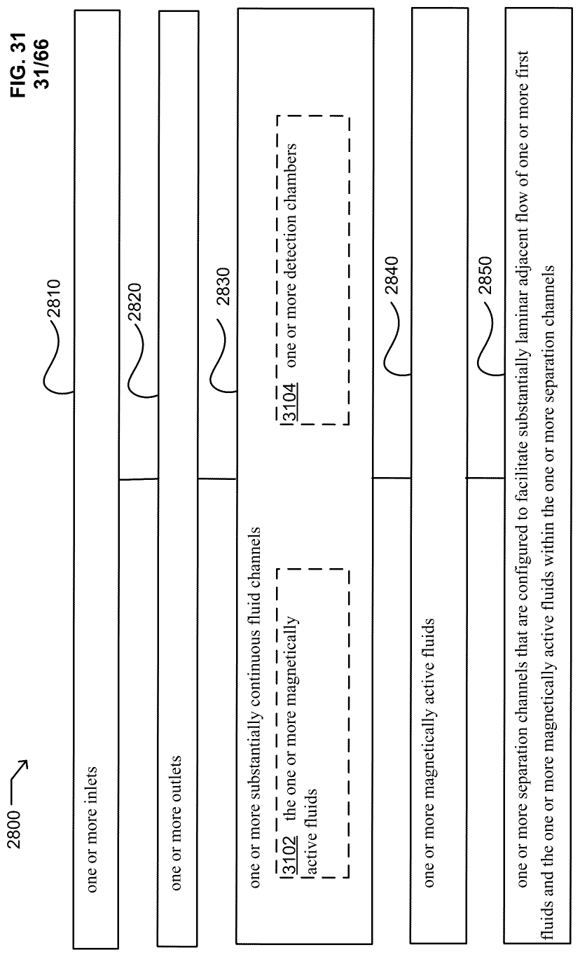

FIG. 31 illustrates alternative embodiments of device 2800 of FIG. 28. FIG. 31 illustrates example embodiments of module 2830. Additional embodiments may include an embodiment 3102, and/or an embodiment 3104.

At embodiment 3102, module 2830 may include the one or more magnetically active fluids. In some embodiments, a device may include one or more magnetically active fluids. In some embodiments, a device may include one or more magnetically active fluids 126 that may include a ferrofluid. In some embodiments, the one or more magnetically active fluids 126 may include paramagnetic particles. In some embodiments, the one or more magnetically active fluids 126 may include diamagnetic particles. In some embodiments, the one or more magnetically active fluids 126 may include magnetic particles. Magnetically active fluid 126 may exhibit numerous characteristics. Examples of such characteristics include, but are not limited to, viscosity, density, miscibility, solubility, vapor pressure, and the like.

At embodiment 3104, module 2830 may include one or more detection chambers. In some embodiments, a device may include one or more detection chambers that are operably associated with one or more separation channels 118. Detection chambers may be configured to detect one or more components through use of numerous technologies. Examples of such technologies include, but are not limited to, spectroscopy, electrochemical detection, polynucleotide detection, fluorescence anisotropy, fluorescence resonance energy transfer, electron transfer, enzyme assay, electrical conductivity, isoelectric focusing, chromatography, immunoprecipitation, immunoseparation, aptamer binding, electrophoresis, use of a CCD camera, immunoassay, polypeptide detection, antibody interaction, chemical interaction, diffusion, filtration, chromatography, aptamer interaction, magnetism, competition assay, or substantially any combination thereof.

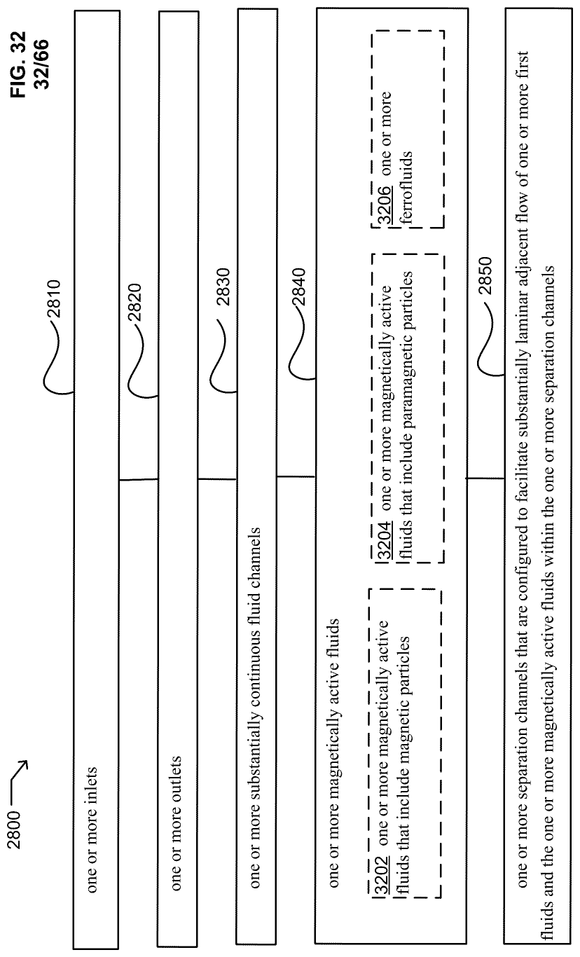

FIG. 32 illustrates alternative embodiments of device 2800 of FIG. 28. FIG. 32 illustrates example embodiments of module 2840. Additional embodiments may include an embodiment 3202, an embodiment 3204, and/or an embodiment 3206.

At embodiment 3202, module 2840 may include one or more magnetically active fluids that include magnetic particles. In some embodiments, one or more devices may include one or more magnetically active fluids 126 that include magnetic particles. In some embodiments, the one or more magnetically active fluids 126 may include a ferrofluid. In some embodiments, the one or more magnetically active fluids 126 may include paramagnetic particles. In some embodiments, the one or more magnetically active fluids 126 may include diamagnetic particles. In some embodiments, the magnetic particles may be coated with a surfactant. Examples of such surfactants include, but are not limited to, oleic acid, tetramethylammonium hydroxide, citric acid, soy lecithin, and the like.

At embodiment 3204, module 2840 may include one or more magnetically active fluids that include paramagnetic particles. In some embodiments, a device may include one or more magnetically active fluids 126 that include paramagnetic particles. Examples of elements and compounds that are paramagnetic include, but are not limited to, aluminum, barium, calcium, oxygen, platinum, sodium, strontium, uranium, magnesium, technetium, dysprosium, copper sulphate, dysprosium oxide, ferric chloride, ferric oxide, holmium oxide, manganese chloride, and the like.

At embodiment 3206, module 2840 may include one or more ferrofluids. In some embodiments, a device may include one or more ferrofluids. Numerous types of ferrofluids may be utilized. For example, in some embodiments, one or more ferrofluids may be used that are suitable biological buffers. Accordingly, the activity and/or integrity of biological materials may be preserved following translocation into such ferrofluids. In some embodiments, ferrofluids may be selected that are matched to one or more sample fluids 104, one or more second separation fluids, or substantially any combination thereof. Ferrofluids may be selected that exhibit numerous characteristics that include, but are not limited to, viscosity, density, miscibility, solvent characteristics, vapor pressure, freezing temperature, and the like.

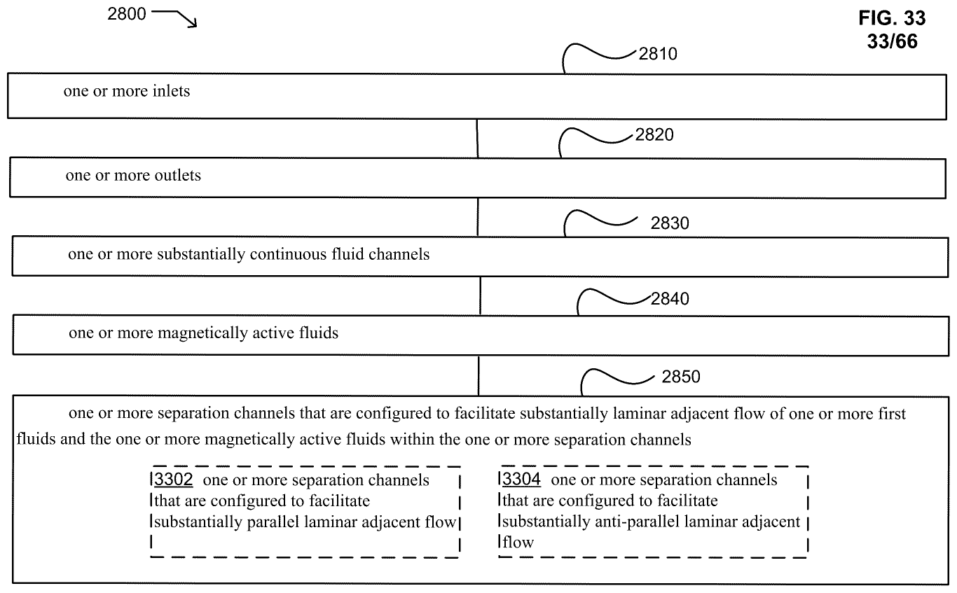

FIG. 33 illustrates alternative embodiments of device 2800 of FIG. 28. FIG. 33 illustrates example embodiments of module 2850. Additional embodiments may include an embodiment 3302, and/or an embodiment 3304.

At embodiment 3302, module 2850 may include one or more separation channels that are configured to facilitate substantially parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially the same direction.

At embodiment 3304, module 2850 may include one or more separation channels that are configured to facilitate substantially anti-parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially opposite directions.

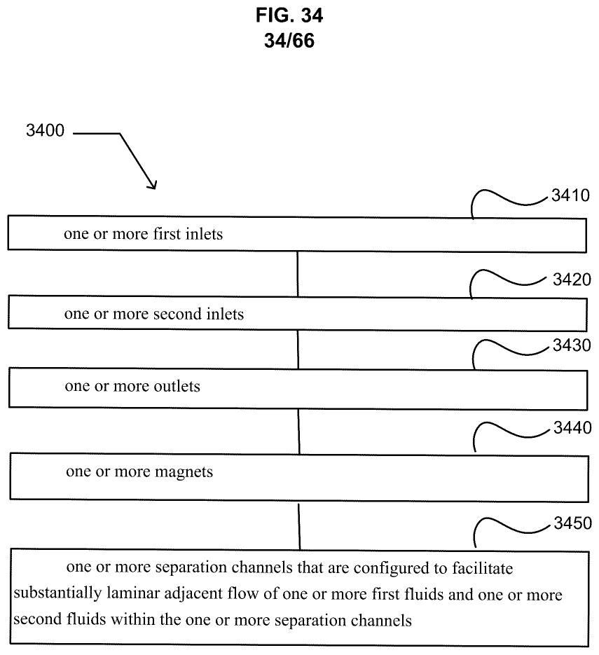

FIG. 34 illustrates a device 3400 representing examples of modules that may be used to perform a method for separating one or more constituents from one or more samples 102. In FIG. 34, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The device 3400 includes module 3410 that includes one or more first inlets. In some embodiments, module 3410 may include one or more first fluid inlets. In some embodiments, module 3410 may include one or more sample fluid inlets.

The device 3400 includes module 3420 that includes one or more second inlets. In some embodiments, module 3420 may include one or more second fluid inlets.

The device 3400 includes module 3430 that includes one or more outlets. In some embodiments, module 3430 may include one or more first fluid outlets. In some embodiments, module 3430 may include one or more second fluid outlets. In some embodiments, module 3430 may include one or more first fluid outlets and one or more second fluid outlets.

The device 3400 includes module 3440 that includes one or more magnets. In some embodiments, module 3440 may include one or more attractive magnets. In some embodiments, module 3440 may include one or more repulsive magnets. In some embodiments, module 3440 may include one or more magnets configured to facilitate translocation of one or more magnetically active components from the one or more first fluids to the one or more second fluids within the one or more separation channels.

The device 3400 includes module 3450 that includes one or more separation channels that are configured to facilitate substantially laminar adjacent flow of one or more first fluids and one or more second fluids within the one or more separation channels. In some embodiments, module 3450 may include one or more separation channels that are configured to facilitate substantially parallel laminar adjacent flow. In some embodiments, module 3450 may include one or more separation channels that are configured to facilitate substantially anti-parallel laminar adjacent flow.

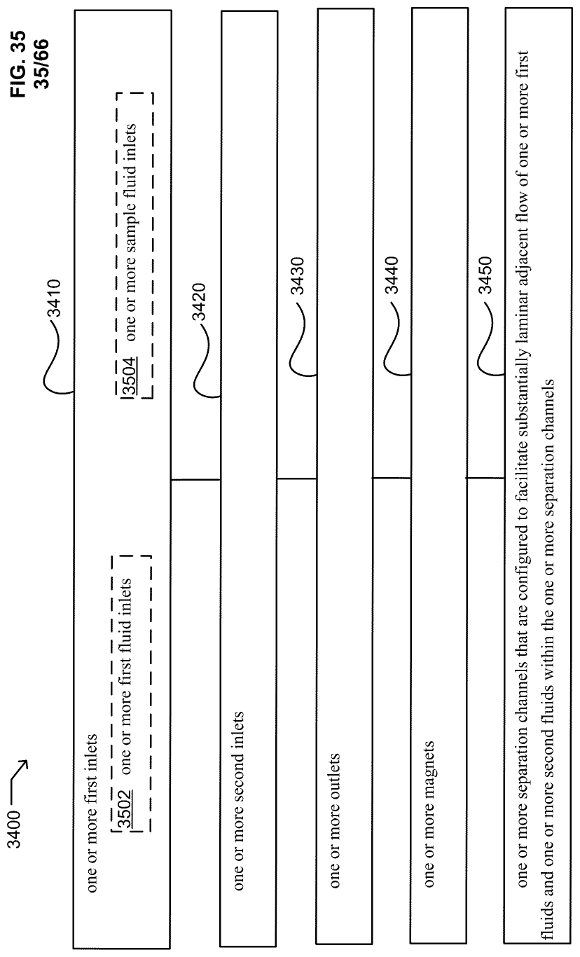

FIG. 35 illustrates alternative embodiments of device 3400 of FIG. 34. FIG. 35 illustrates example embodiments of module 3410. Additional embodiments may include an embodiment 3502, and/or an embodiment 3504.

At embodiment 3502, module 3410 may include one or more first fluid inlets. In some embodiments, a device may include one or more first fluid inlets 112 that are operably associated with one or more separation channels 118. Fluid inlets may be configured in numerous ways. For example, in some embodiments, a fluid inlet may be configured to include one or more septa through which one or more first fluids 120 may be injected through use of a syringe. In some embodiments, a fluid inlet may be configured to include one or more fittings (e.g., leur lock fittings) through which one or more first fluids 120 may be injected through use of a syringe. In some embodiments, a fluid inlet may be configured to include one or more fittings to which one or more pumps may be attached to pump one or more sample fluids 104 into the device.

At embodiment 3504, module 3410 may include one or more sample fluid inlets. In some embodiments, a device may include one or more sample fluid inlets that are operably associated with one or more separation channels 118. Sample fluid inlets may be configured in numerous ways. For example, in some embodiments, a sample fluid inlet may be configured to include one or more septa through which one or more sample fluids 104 may be injected through use of a syringe. In some embodiments, a sample fluid inlet may be configured to include one or more fittings (e.g., leur lock fittings) through which one or more sample fluids 104 may be injected through use of a syringe. In some embodiments, a sample fluid inlet may be configured to include one or more fittings to which one or more pumps may be attached to pump one or more sample fluids 104 into the device.

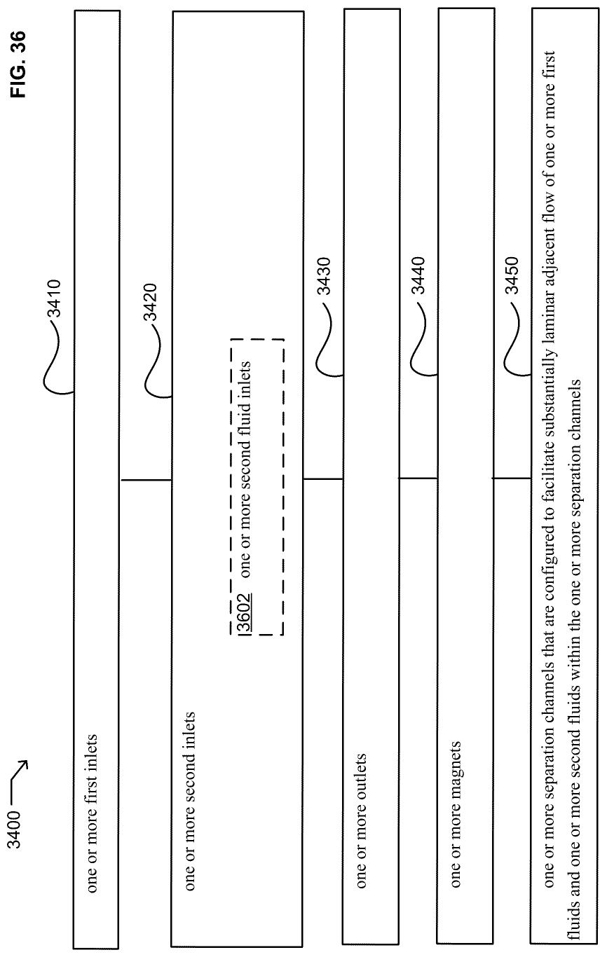

FIG. 36 illustrates alternative embodiments of device 3400 of FIG. 34. FIG. 36 illustrates example embodiments of module 3420. Additional embodiments may include an embodiment 3602.

At embodiment 3602, module 3420 may include one or more second fluid inlets. In some embodiments, a device may include one or more second fluid inlets 114 that are operably associated with one or more separation channels 118. Second fluid inlets 114 may be configured in numerous ways. For example, in some embodiments, a second fluid inlet may be configured to include one or more septa through which one or more second fluids 122 may be injected through use of a syringe. In some embodiments, a second fluid inlet 114 may be configured to include one or more fittings (e.g., leur lock fittings) through which one or more second fluids 122 may be injected through use of a syringe. In some embodiments, a second fluid inlet 114 may be configured to include one or more fittings to which one or more pumps may be attached to pump one or more second fluids 122 into the device.

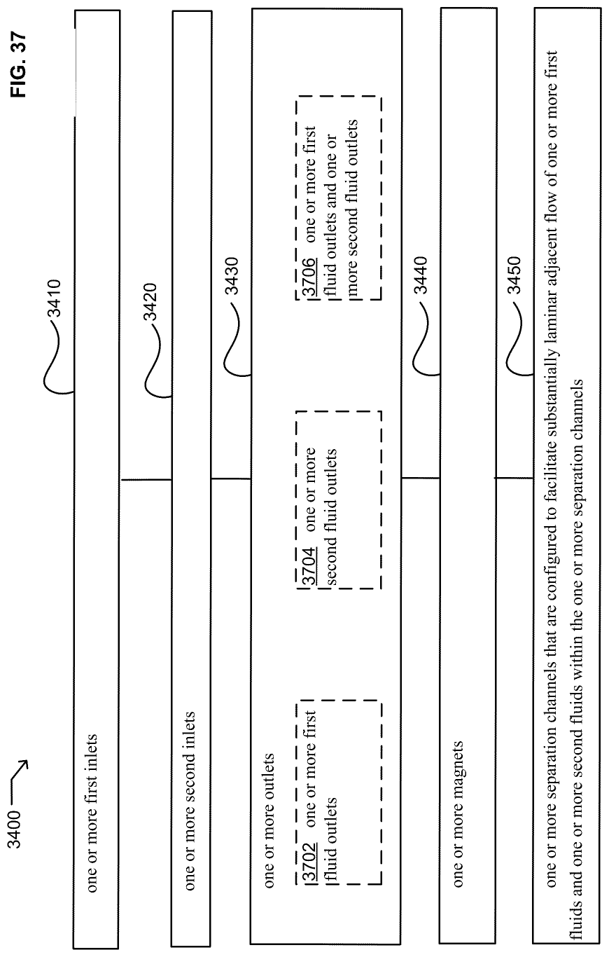

FIG. 37 illustrates alternative embodiments of device 3400 of FIG. 34. FIG. 37 illustrates example embodiments of module 3430. Additional embodiments may include an embodiment 3702, an embodiment 3704, and/or an embodiment 3706.

At embodiment 3702, module 3430 may include one or more first fluid outlets. In some embodiments, a device may include one or more first fluid outlets 116 that are operably associated with one or more separation channels 118. In some embodiments, a device may include one or more first fluid outlets 116 that are operably associated with one or more substantially continuous fluid channels 128.

At embodiment 3704, module 3430 may include one or more second fluid outlets. In some embodiments, a device may include one or more second fluid outlets 116 that are operably associated with one or more separation channels 118. In some embodiments, a device may include one or more second fluid outlets 116 that are operably associated with one or more substantially continuous fluid channels 128.

At embodiment 3706, module 3430 may include one or more first fluid outlets and one or more second fluid outlets. In some embodiments, a device may include one or more first fluid outlets 116 and one or more second fluid outlets 116 that are operably associated with one or more separation channels 118. In some embodiments, a device may include one or more first fluid outlets 116 and one or more second fluid outlets 116 that are each operably associated with one or more substantially continuous fluid channels 128. In some embodiments, such devices provide for continuous separation of one or more constituents from one or more samples 102.

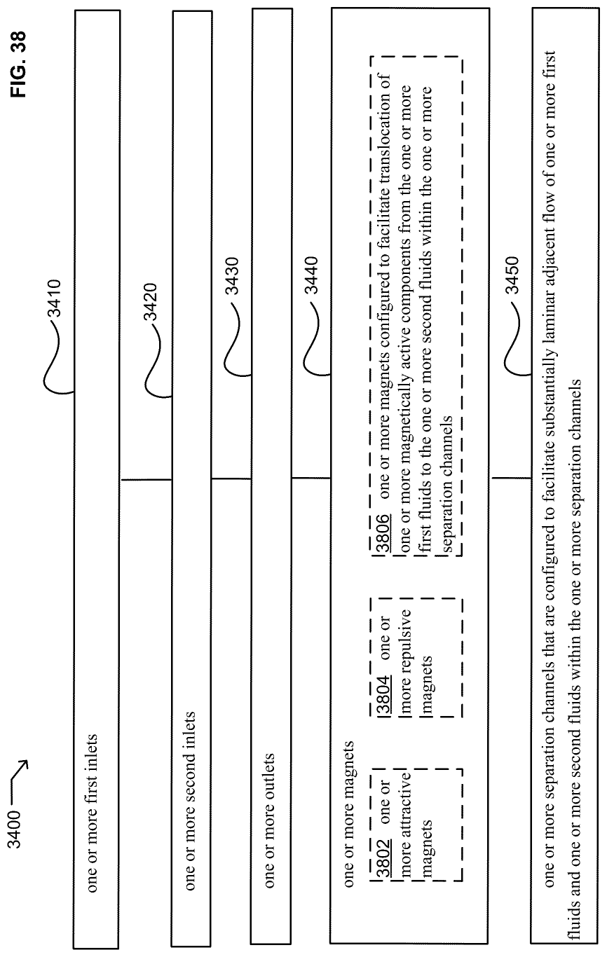

FIG. 38 illustrates alternative embodiments of device 3400 of FIG. 34. FIG. 38 illustrates example embodiments of module 3440. Additional embodiments may include an embodiment 3802, an embodiment 3804, and/or an embodiment 3806.

At embodiment 3802, module 3440 may include one or more attractive magnets. In some embodiments, a device may include one or more attractive magnets 124. In some embodiments, the one or more attractive magnets 124 may include one or more permanent magnets 124. In some embodiments, the one or more attractive magnets 124 may include one or more electromagnets 124. In some embodiments, the one or more attractive magnets 124 facilitate translocation of one or more magnetically active constituents 106 through magnetic attraction.

At embodiment 3804, module 3440 may include one or more repulsive magnets. In some embodiments, a device may include one or more repulsive magnets 124. In some embodiments, the one or more repulsive magnets 124 may include one or more permanent magnets 124. In some embodiments, the one or more repulsive magnets 124 may include one or more electromagnets. In some embodiments, the one or more repulsive magnets 124 facilitate translocation of one or more magnetically active constituents 106 through magnetic repulsion.

At embodiment 3806, module 3440 may include one or more magnets configured to facilitate translocation of one or more magnetically active components from the one or more first fluids to the one or more second fluids within the one or more separation channels. In some embodiments, a device may include one or more magnets 124 configured to facilitate translocation of one or more magnetically active components from the one or more first fluids 120 to the one or more second fluids 122 within the one or more separation channels 118. In some embodiments, the one or more magnets 124 may utilize magnetic attraction to facilitate translocation of one or more magnetically active components from the one or more first fluids 120 to the one or more second fluids 122 within the one or more separation channels 118. In some embodiments, the one or more magnets 124 may utilize magnetic repulsion to facilitate translocation of one or more magnetically active components from the one or more first fluids 120 to the one or more second fluids 122 within the one or more separation channels 118. In some embodiments, the one or more magnets 124 may include one or more permanent magnets 124. In some embodiments, the one or more magnets 124 may include one or more electromagnets.

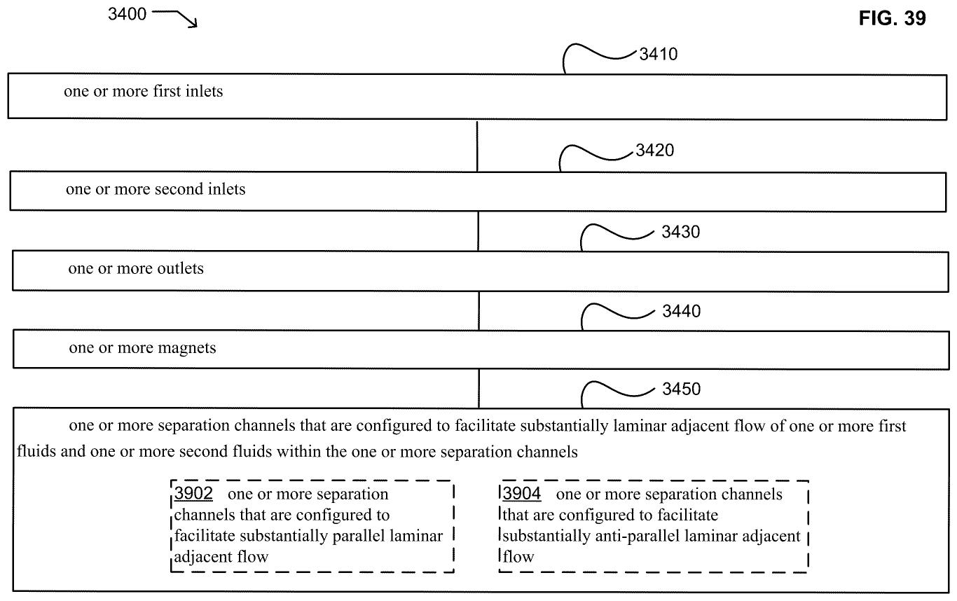

FIG. 39 illustrates alternative embodiments of device 3400 of FIG. 34. FIG. 39 illustrates example embodiments of module 3450. Additional embodiments may include an embodiment 3902, and/or an embodiment 3904.

At embodiment 3902, module 3450 may include one or more separation channels that are configured to facilitate substantially parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially the same direction.

At embodiment 3904, module 3450 may include one or more separation channels that are configured to facilitate substantially anti-parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially opposite directions.

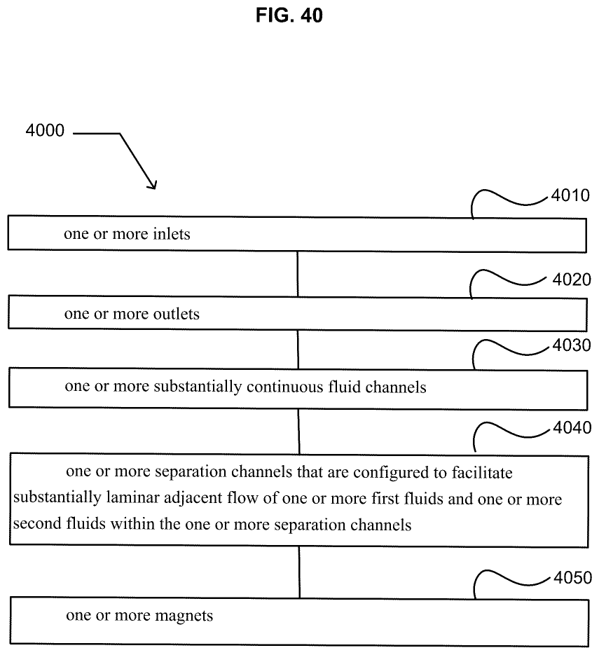

FIG. 40 illustrates a device 4000 representing examples of modules that may be used to perform a method for separating one or more constituents from one or more samples 102. In FIG. 40, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The device 4000 includes module 4010 that includes one or more inlets. In some embodiments, module 4010 may include one or more first fluid inlets. In some embodiments, module 4010 may include one or more sample fluid inlets.

The device 4000 includes module 4020 that includes one or more outlets. In some embodiments, module 4020 may include one or more first fluid outlets. In some embodiments, module 4020 may include one or more sample fluid outlets. In some embodiments, module 4020 may include one or more first fluid outlets and one or more magnetically active fluid outlets.

The device 4000 includes module 4030 that includes one or more substantially continuous fluid channels. In some embodiments, module 4030 may include one or more extraction fluids. In some embodiments, module 4030 may include one or more detection chambers.

The device 4000 includes module 4040 that includes one or more separation channels that are configured to facilitate substantially laminar adjacent flow of one or more first fluids and one or more second fluids within the one or more separation channels. In some embodiments, module 4040 may include one or more separation channels that are configured to facilitate substantially parallel laminar adjacent flow. In some embodiments, module 4040 may include one or more separation channels that are configured to facilitate substantially anti-parallel laminar adjacent flow.

The device 4000 includes module 4050 that includes one or more magnets. In some embodiments, module 4050 may include one or more magnets that are configured to facilitate translocation of one or more magnetically active components from the one or more first fluids to the one or more magnetically active fluids. In some embodiments, module 4050 may include one or more attractive magnets. In some embodiments, module 4050 may include one or more repulsive magnets.

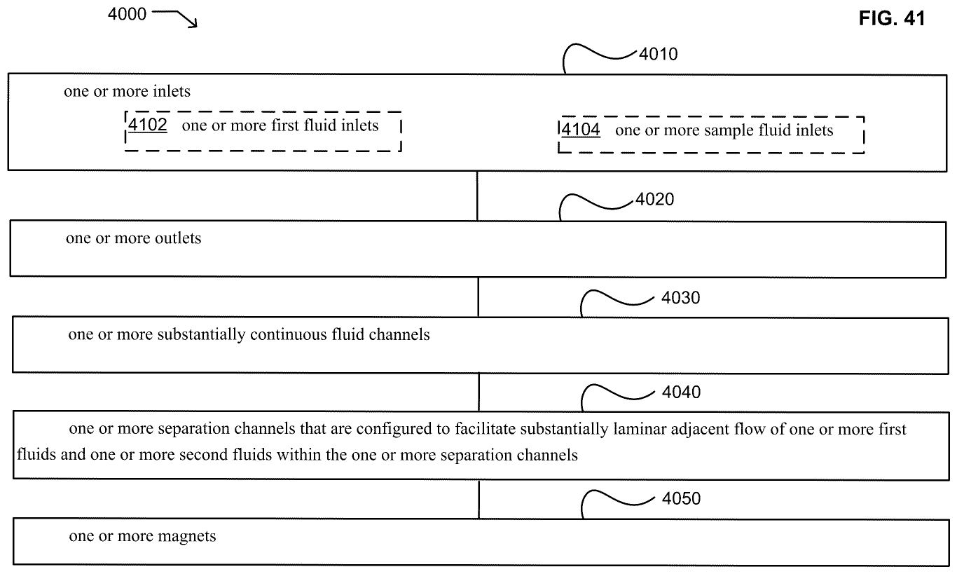

FIG. 41 illustrates alternative embodiments of device 4000 of FIG. 40. FIG. 41 illustrates example embodiments of module 4010. Additional embodiments may include an embodiment 4102, and/or an embodiment 4104.

At embodiment 4102, module 4010 may include one or more first fluid inlets. In some embodiments, a device may include one or more first fluid inlets 112 that are operably associated with one or more separation channels 118. Fluid inlets may be configured in numerous ways. For example, in some embodiments, a fluid inlet may be configured to include one or more septa through which one or more first fluids 120 may be injected through use of a syringe. In some embodiments, a fluid inlet may be configured to include one or more fittings (e.g., leur lock fittings) through which one or more first fluids 120 may be injected through use of a syringe. In some embodiments, a fluid inlet may be configured to include one or more fittings to which one or more pumps may be attached to pump one or more sample fluids 104 into the device.

At embodiment 4104, module 4010 may include one or more sample fluid inlets. In some embodiments, a device may include one or more sample fluid inlets that are operably associated with one or more separation channels 118. Sample fluid inlets may be configured in numerous ways. For example, in some embodiments, a sample fluid 104 inlet may be configured to include one or more septa through which one or more sample fluids 104 may be injected through use of a syringe. In some embodiments, a sample fluid 104 inlet may be configured to include one or more fittings (e.g., leur lock fittings) through which one or more sample fluids 104 may be injected through use of a syringe. In some embodiments, a sample fluid 104 inlet may be configured to include one or more fittings to which one or more pumps may be attached to pump one or more sample fluids 104 into the device.

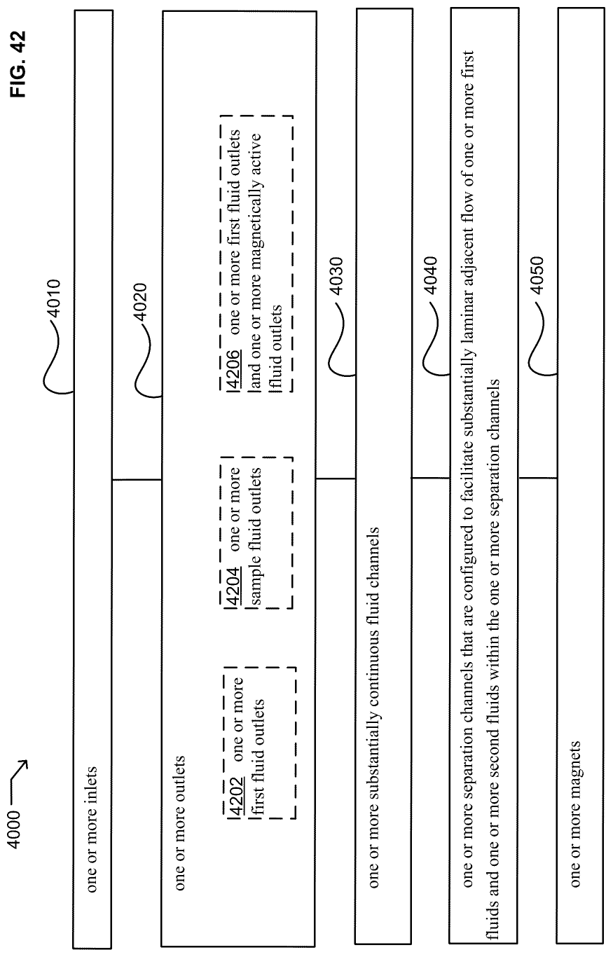

FIG. 42 illustrates alternative embodiments of device 4000 of FIG. 40. FIG. 42 illustrates example embodiments of module 4020. Additional embodiments may include an embodiment 4202, an embodiment 4204, and/or an embodiment 4206.

At embodiment 4202, module 4020 may include one or more first fluid outlets. In some embodiments, a device may include one or more first fluid outlets 116 that are operably associated with one or more separation channels 118. In some embodiments, a device may include one or more first fluid outlets 116 that are operably associated with one or more substantially continuous fluid channels 128.

At embodiment 4204, module 4020 may include one or more sample fluid outlets. In some embodiments, a device may include one or more sample fluid outlets that are operably associated with one or more separation channels 118. In some embodiments, a device may include one or more sample fluid outlets that are operably associated with one or more substantially continuous fluid channels 128. In some embodiments, a device may include one or more sample fluid outlets that are operably associated with one or more additional separation channels 118.

At embodiment 4206, module 4020 may include one or more first fluid outlets and one or more magnetically active fluid outlets. In some embodiments, a device may include one or more first fluid outlets 116 and one or more magnetically active fluid outlets 116 that are operably associated with one or more separation channels 118. In some embodiments, a device may include one or more magnetically active fluid outlets 116 that are operably associated with one or more substantially continuous fluid channels 128. In some embodiments, such devices may be configured for continuous separation of components from one or more sample fluids 104.

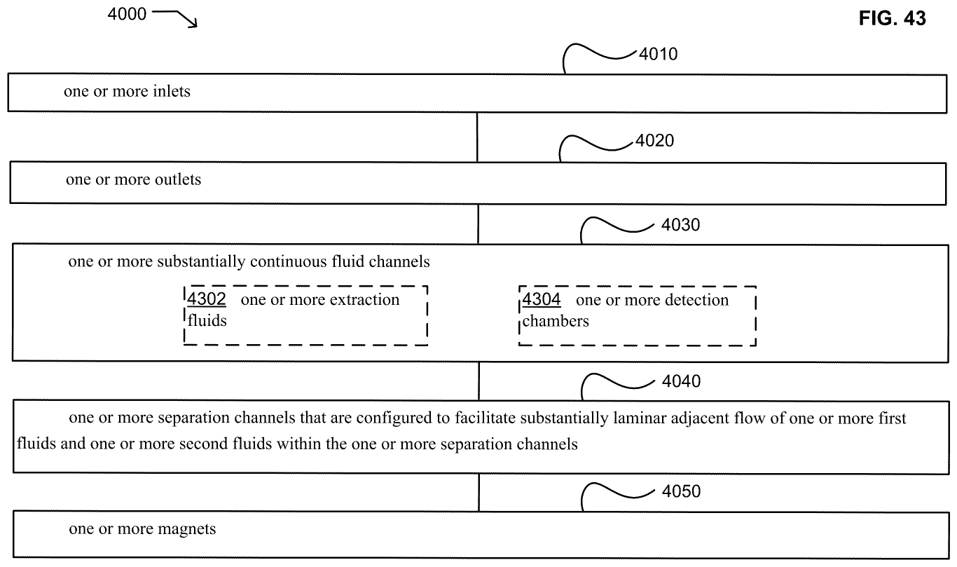

FIG. 43 illustrates alternative embodiments of device 4000 of FIG. 40. FIG. 43 illustrates example embodiments of module 4030. Additional embodiments may include an embodiment 4302, and/or an embodiment 4304.

At embodiment 4302, module 4030 may include one or more extraction fluids. In some embodiments, a device may include one or more extraction fluids. One or more devices may include numerous types of extraction fluids. Examples of extraction fluids include, but are not limited to, solvents, buffers, acids, bases, and the like.

At embodiment 4304, module 4030 may include one or more detection chambers. In some embodiments, a device may include one or more detection chambers that are operably associated with one or more separation channels 118. Detection chambers may be configured to detect one or more components through use of numerous technologies. Examples of such technologies include, but are not limited to, spectroscopy, electrochemical detection, polynucleotide detection, fluorescence anisotropy, fluorescence resonance energy transfer, electron transfer, enzyme assay, electrical conductivity, isoelectric focusing, chromatography, immunoprecipitation, immunoseparation, aptamer binding, electrophoresis, use of a CCD camera, immunoassay, polypeptide detection, antibody interaction, chemical interaction, diffusion, filtration, aptamer interaction, magnetism, competition assay, or substantially any combination thereof.

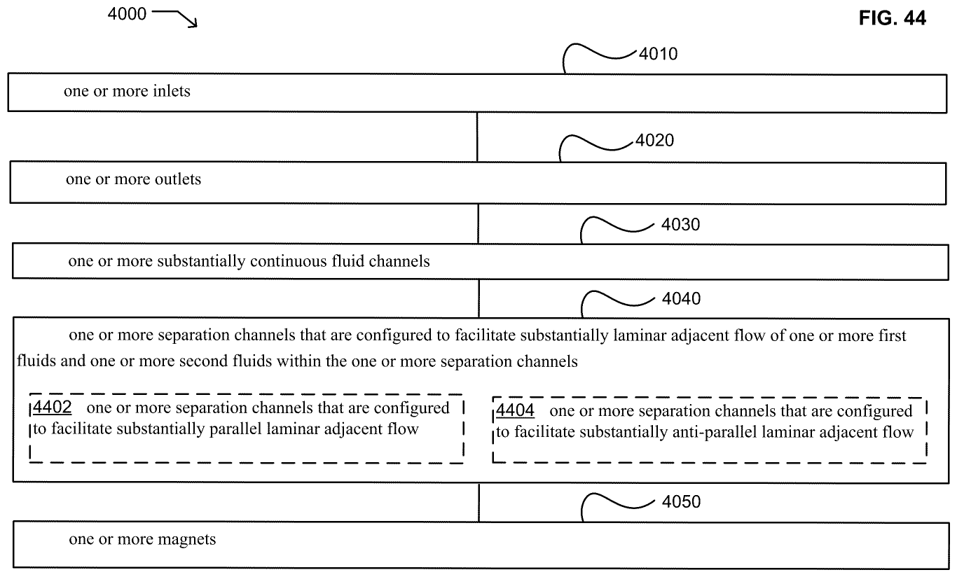

FIG. 44 illustrates alternative embodiments of device 4000 of FIG. 40. FIG. 44 illustrates example embodiments of module 4040. Additional embodiments may include an embodiment 4402, and/or an embodiment 4404.

At embodiment 4402, module 4040 may include one or more separation channels that are configured to facilitate substantially parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially the same direction.

At embodiment 4404, module 4040 may include one or more separation channels that are configured to facilitate substantially anti-parallel laminar adjacent flow. In some embodiments, one or more devices may include one or more separation channels 118 that are configured to facilitate substantially parallel laminar adjacent flow of one or more first fluids 120 and one or more magnetically active fluids 126 within one or more separation channels 118. In such embodiments, the one or more first fluids 120 and the one or more magnetically active fluids 126 flow in substantially opposite directions.

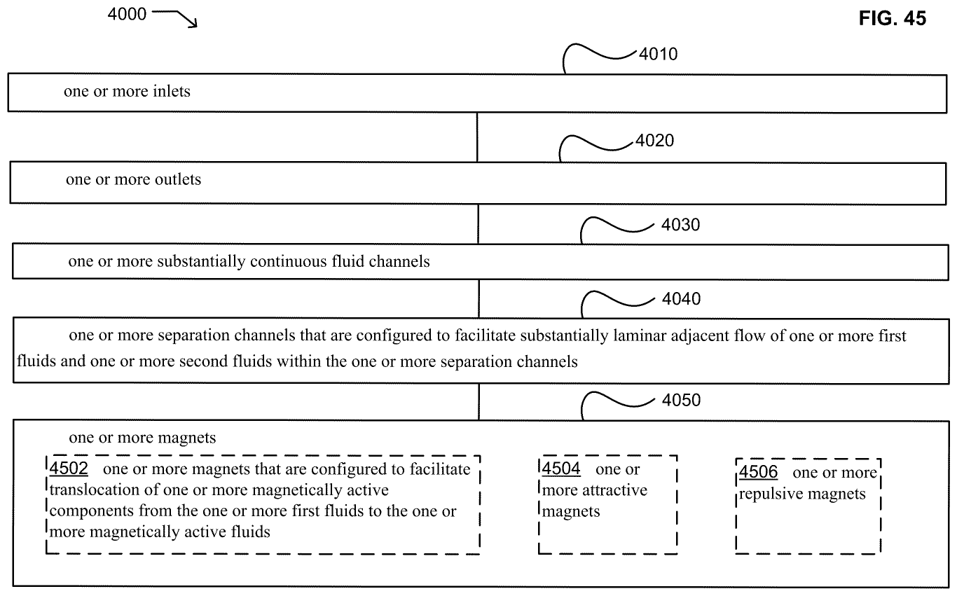

FIG. 45 illustrates alternative embodiments of device 4000 of FIG. 40. FIG. 45 illustrates example embodiments of module 4050. Additional embodiments may include an embodiment 4502, an embodiment 4504, and/or an embodiment 4506.

At embodiment 4502, module 4050 may include one or more magnets that are configured to facilitate translocation of one or more magnetically active components from the one or more first fluids to the one or more magnetically active fluids. In some embodiments, a device may include one or more magnets 124 that are configured to facilitate translocation of one or more magnetically active components from one or more first fluids 120 to one or more magnetically active fluids 126. In some embodiments, the one or more magnets 124 may be configured to utilize magnetic attraction. In some embodiments, the one or more magnets 124 may be configured to utilize magnetic repulsion.

At embodiment 4504, module 4050 may include one or more attractive magnets. In some embodiments, a device may include one or more attractive magnets 124. In some embodiments, the one or more attractive magnets 124 may include one or more permanent magnets 124. In some embodiments, the one or more attractive magnets 124 may include one or more electromagnets 124. In some embodiments, the one or more attractive magnets 124 facilitate translocation of one or more magnetically active constituents 106 through magnetic attraction.

At embodiment 4506, module 4050 may include one or more repulsive magnets. In some embodiments, a device may include one or more repulsive magnets 124. In some embodiments, the one or more repulsive magnets 124 may include one or more permanent magnets 124. In some embodiments, the one or more repulsive magnets 124 may include one or more electromagnets 124. In some embodiments, the one or more repulsive magnets 124 facilitate translocation of one or more magnetically active constituents 106 through magnetic repulsion.

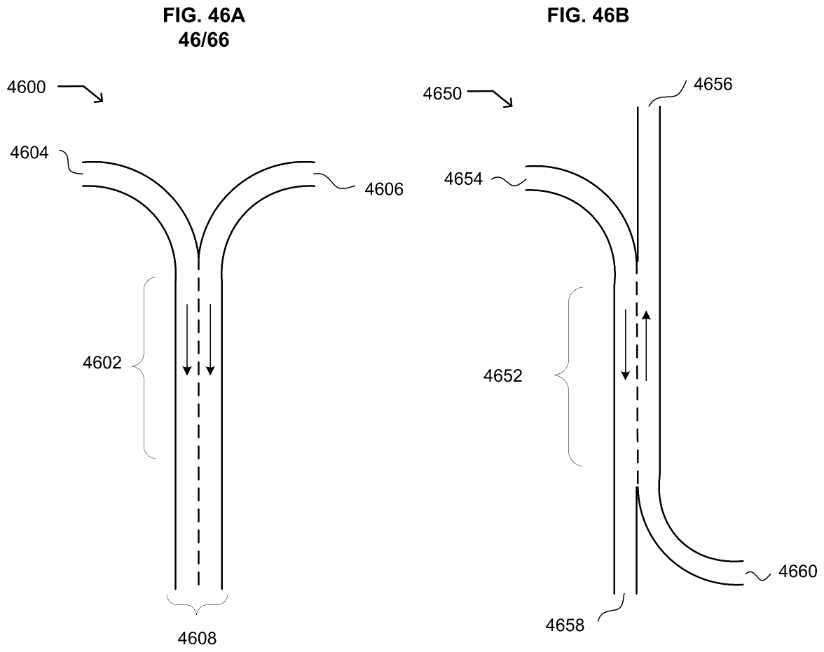

FIG. 46A illustrates a separation channel 4602 in which a first fluid 120 and a second fluid 122 are in substantially parallel flow. The first fluid may enter into the separation channel 4602 through a first fluid inlet 4604 and the second fluid may enter into the separation channel 4602 through a second fluid inlet 4606. The first fluid 120 and the second fluid 122 may exit the separation channel 4602 through a fluid outlet 4608.

FIG. 46B illustrates a separation channel 4652 in which a first fluid 120 and a second fluid 122 are in substantially anti-parallel flow. The first fluid 120 may enter into the separation channel 4652 through a first fluid inlet 4654 and the second fluid 122 may enter into the separation channel 4652 through a second fluid inlet 4660. The first fluid 120 may exit the separation channel 4652 through a first fluid outlet 4658. The second fluid 122 may exit the separation channel 4652 through a second fluid outlet 4656.

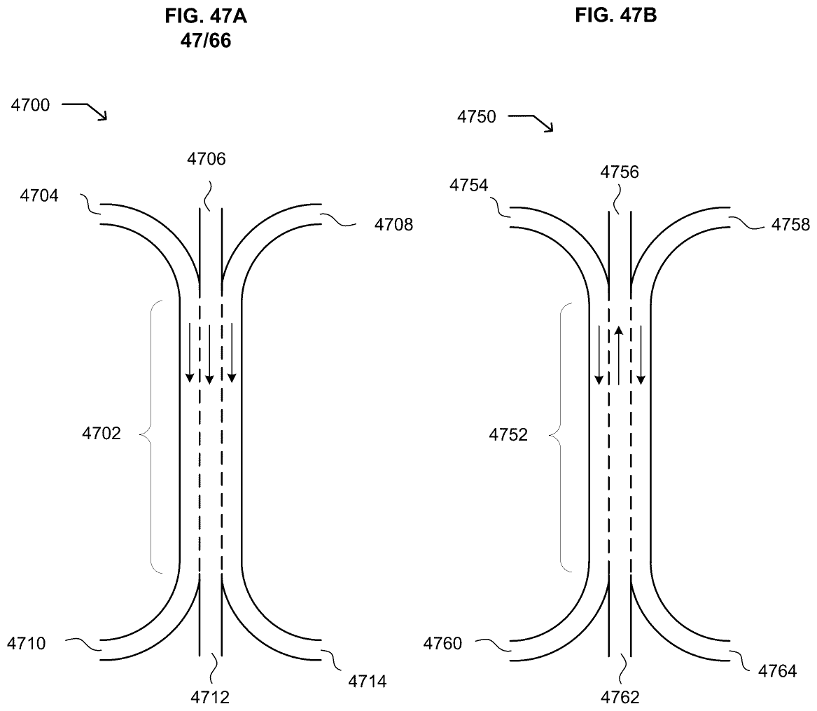

FIG. 47A illustrates a separation channel 4702 in which a first fluid 120, a second fluid 122, and a magnetically active fluid 126 are in substantially parallel flow. The first fluid 120 may enter into the separation channel 4702 through a first fluid inlet 4704, the second fluid 122 may enter into the separation channel 4702 through a second fluid inlet 4708, and the magnetically active fluid 126 may enter into the separation channel 4702 through a magnetically active fluid inlet 4706. The first fluid may exit the separation channel 4702 through a first fluid outlet 4710. The second fluid may exit the separation channel 4702 through a second fluid outlet 4714. The magnetically active fluid 126 may exit the separation channel 4702 through a magnetically active fluid outlet 4712.

FIG. 47B illustrates a separation channel 4752 in which a first fluid 120, a second fluid 122, and a magnetically active fluid 126 are in substantially anti-parallel flow. The first fluid 120 may enter into the separation channel 4752 through a first fluid inlet 4754, the second fluid 122 may enter into the separation channel 4752 through a second fluid inlet 4758, and the magnetically active fluid 126 may enter into the separation channel 4752 through a magnetically active fluid inlet 4762. The first fluid 120 may exit the separation channel 4752 through a first fluid outlet 4760. The second fluid 122 may exit the separation channel 4752 through a second fluid outlet 4764. The magnetically active fluid 126 may exit the separation channel 4752 through a magnetically active fluid outlet 4756.

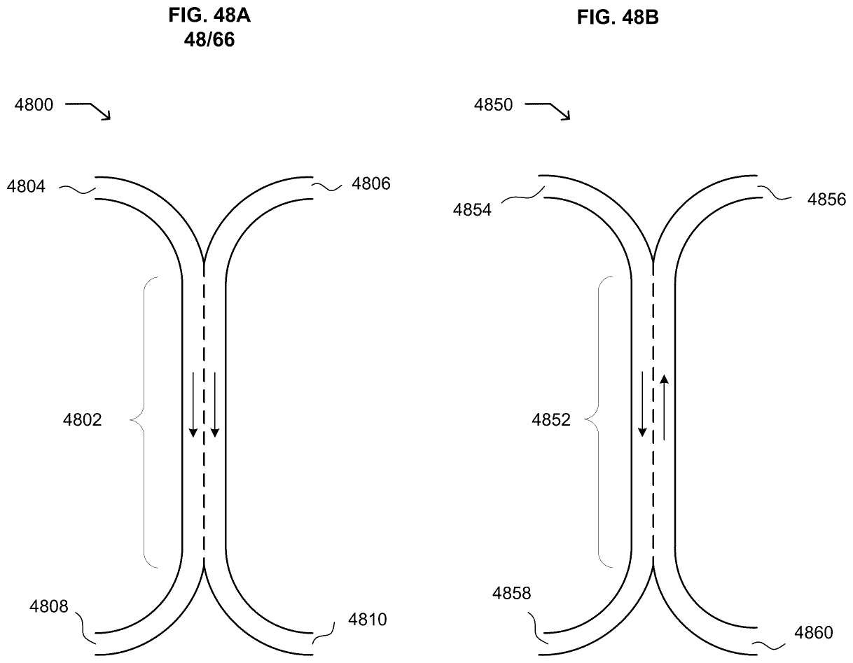

FIG. 48A illustrates a separation channel 4802 in which a first fluid 120 and a second fluid 122 are in substantially parallel flow. The first fluid 120 may enter into the separation channel 4802 through a first fluid inlet 4804 and the second fluid 122 may enter into the separation channel 4802 through a second fluid inlet 4806. The first fluid 120 may exit the separation channel 4802 through a first fluid outlet 4808 and the second fluid 122 may exit the separation channel 4802 through a second fluid outlet 4810.

FIG. 48B illustrates a separation channel 4852 in which a first fluid 120 and a second fluid 122 are in substantially anti-parallel flow. The first fluid 120 may enter into the separation channel 4852 through a first fluid inlet 4854 and the second fluid 122 may enter into the separation channel 4852 through a second fluid inlet 4860. The first fluid 120 may exit the separation channel 4852 through a first fluid outlet 4858 and the second fluid 122 may exit the separation channel 4852 through a second fluid outlet 4856.

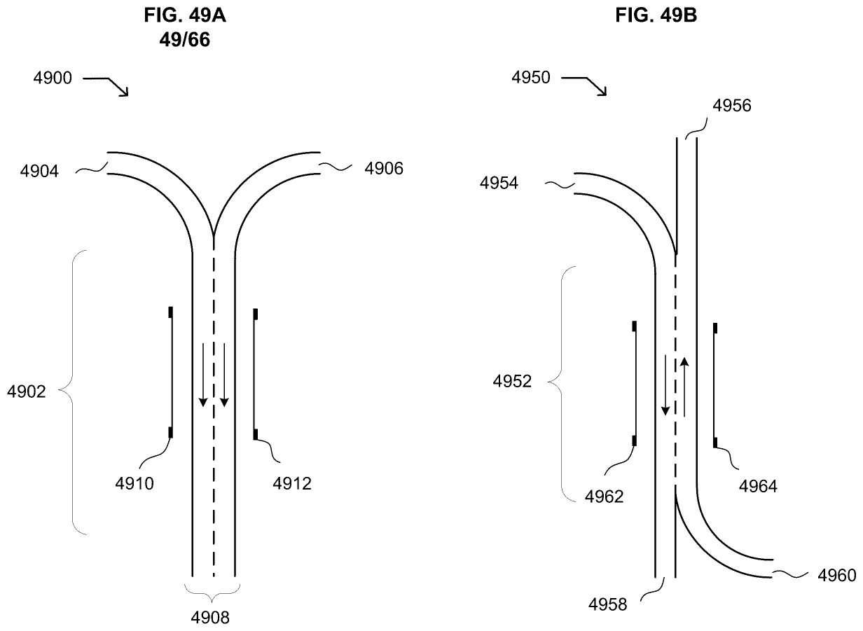

FIG. 49A illustrates a separation channel 4902 in which a first fluid 120 and a second fluid 122 are in substantially parallel flow. The first fluid 120 may enter into the separation channel 4902 through a first fluid inlet 4904 and the second fluid 122 may enter into the separation channel through a second fluid inlet 4906. The first fluid 120 and the second fluid 122 may exit the separation channel 4902 through a fluid outlet 4908. A first magnet 4910 and a second magnet 4912 are illustrated and may be present or absent in any combination.

FIG. 49B illustrates a separation channel 4952 in which a first fluid 120 and a second fluid 122 are in substantially anti-parallel flow. The first fluid 120 may enter into the separation channel 4952 through a first fluid inlet 4954 and the second fluid 122 may enter into the separation channel 4952 through a second fluid inlet 4960. The first fluid 120 may exit the separation channel 4952 through a first fluid outlet 4958. The second fluid 122 may exit the separation channel 4952 through a second fluid outlet 4956. A first magnet 4962 and a second magnet 4964 are illustrated and may be present or absent in any combination.

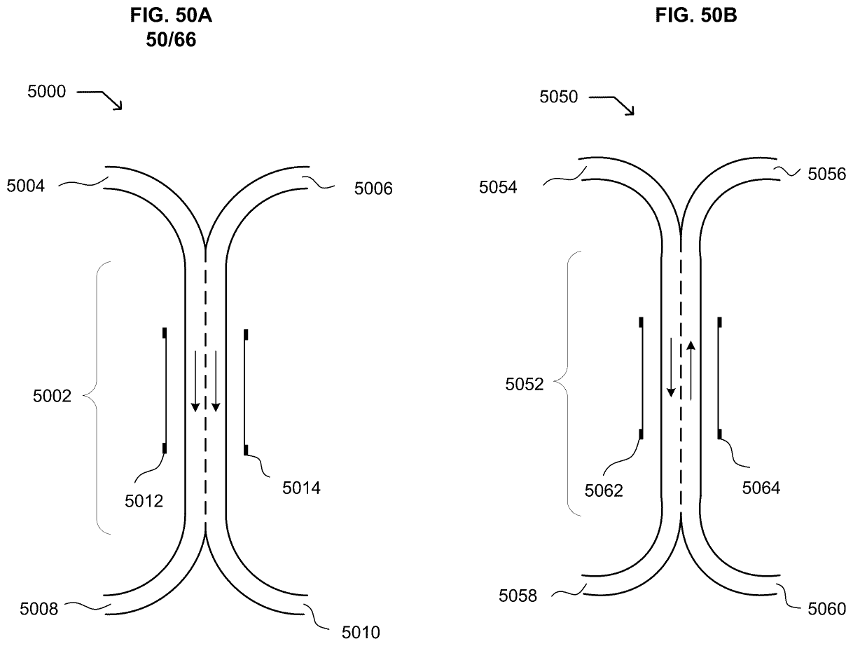

FIG. 50A illustrates a separation channel 5002 in which a first fluid 120 and a second fluid 122 are in substantially parallel flow. The first fluid 120 may enter into the separation channel 5002 through a first fluid inlet 5004 and the second fluid 122 may enter into the separation channel 5002 through a second fluid inlet 5006. The first fluid 120 may exit the separation channel 5002 through a first fluid outlet 5008 and the second fluid 122 may exit the separation channel 5002 through a second fluid outlet 5010. A first magnet 5012 and a second magnet 5014 are illustrated and may be present or absent in any combination.

FIG. 50B illustrates a separation channel 5052 in which a first fluid 120 and a second fluid 122 are in substantially anti-parallel flow. The first fluid 120 may enter into the separation channel 5052 through a first fluid inlet 5054 and the second fluid 122 may enter into the separation channel 5052 through a second fluid inlet 5060. The first fluid 120 may exit the separation channel 5052 through a first fluid outlet 5058 and the second fluid 122 may exit the separation channel 5052 through a second fluid outlet 5056. A first magnet 5062 and a second magnet 5064 are illustrated and may be present or absent in any combination.

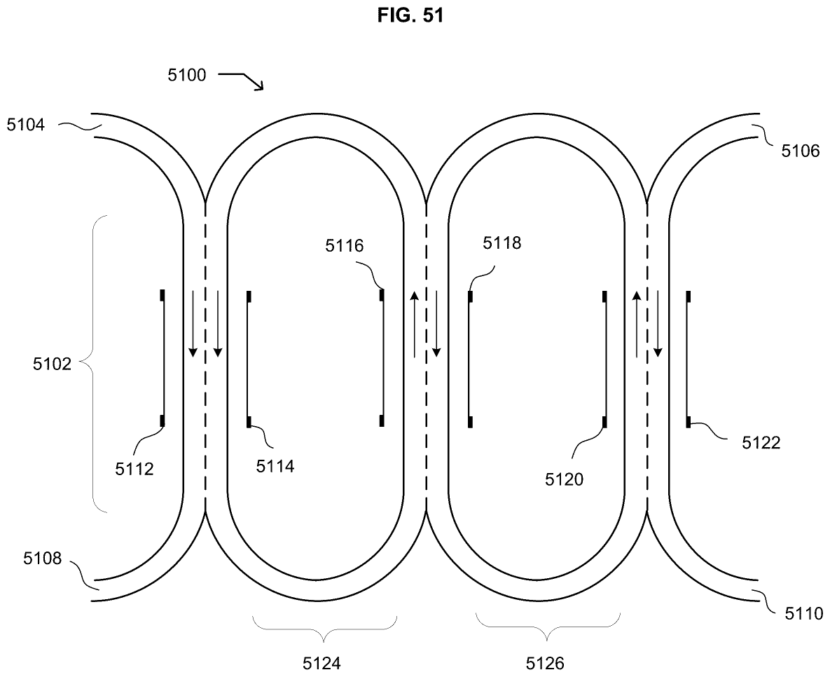

FIG. 51 illustrates a series of separation channels 5102 in which fluids are illustrated in substantially parallel flow and in substantially anti-parallel flow. A first fluid 120 may enter into a first separation channel 5102 of the series through a first fluid inlet 5104 and the second fluid 122 may enter into one of the separation channels of the series 5102 through a second fluid inlet 5106. The first fluid 120 may exit one of the separation channels of the series 5102 through a first fluid outlet 5108 and the second fluid 122 may exit one of the separation channels of the series 5102 through a second fluid outlet 5110. A series of magnets (5112, 5114, 5116, 5118, 5120, and 5122) are illustrated and may be present or absent in any combination. Also illustrated are two substantially continuous channels (5124 and 5126).

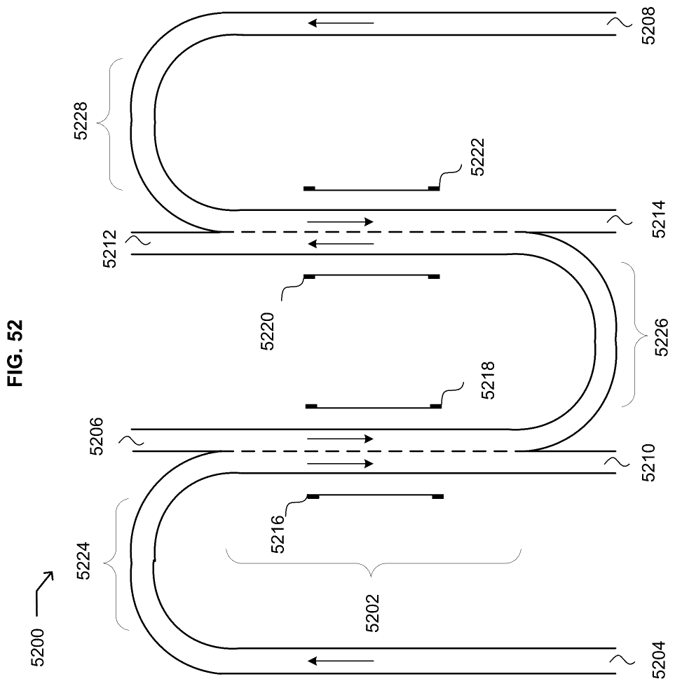

FIG. 52 illustrates a series of separation channels 5202 in which fluids are illustrated in substantially parallel flow and in substantially anti-parallel flow. A first fluid 120 may enter into a first separation channel 5202 of the series through a first fluid inlet 5204, a separation fluid may enter into one of the separation channels of the series 5202 through a separation fluid inlet 5206, and a second fluid 122 may enter into one of the separation channels of the series 5202 through a second fluid inlet 5208. The first fluid 120 may exit one of the separation channels of the series 5202 through a first fluid outlet 5210, a separation fluid may exit one of the separation channels of the series 5202 through a separation fluid outlet 5212, and the second fluid 122 may exit one of the separation channels of the series 5202 through a second fluid outlet 5214. A series of magnets (5216, 5218, 5220, and 5222) are illustrated and may be present or absent in any combination. Also illustrated are three substantially continuous channels (5224, 5226, and 5228).

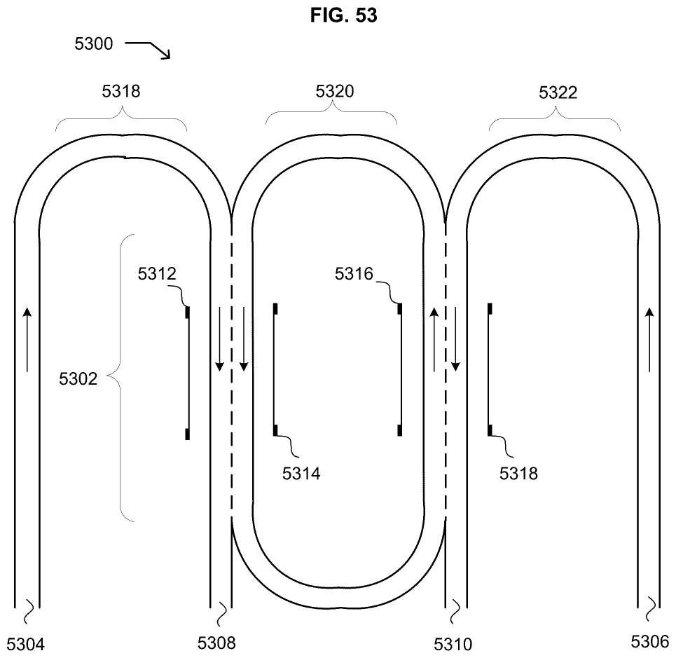

FIG. 53 illustrates a series of separation channels 5302 in which fluids are illustrated in substantially parallel flow and in substantially anti-parallel flow. A first fluid 120 may enter into a first separation channel 5302 of the series through a first fluid inlet 5304 and a second fluid 122 may enter into one of the separation channels of the series 5302 through a second fluid inlet 5306. The first fluid 120 may exit one of the separation channels of the series 5302 through a first fluid outlet 5308 and the second fluid 122 may exit one of the separation channels of the series 5302 through a second fluid outlet 5310. A series of magnets (5312, 5314, 5316, and 5318) are illustrated and may be present or absent in any combination. Also illustrated are three substantially continuous channels (5318, 5320, and 5322). A separation fluid may flow through the substantially continuous channel 5320.

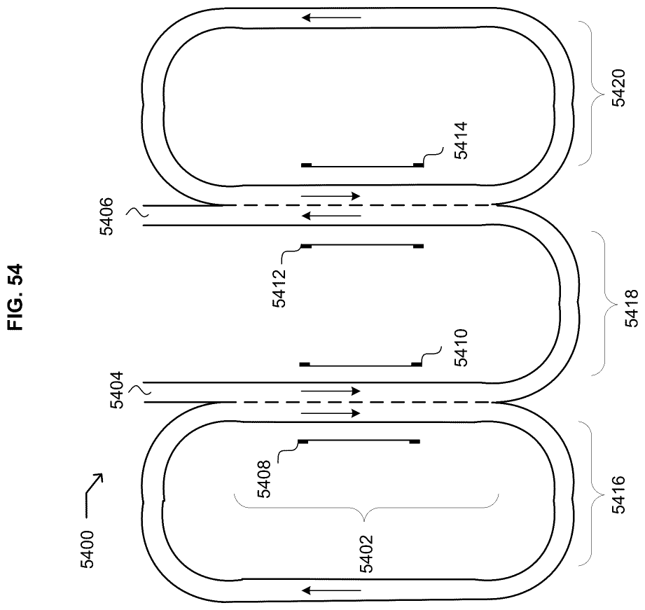

FIG. 54 illustrates a series of separation channels 5402 in which fluids are illustrated in substantially parallel flow and in substantially anti-parallel flow. A first fluid 120 may enter into a first separation channel 5402 of the series through a first fluid inlet 5404. The first fluid 120 may exit one of the separation channels of the series 5402 through a first fluid outlet 5406. A series of magnets (5408, 5410, 5412, and 5414) are illustrated and may be present or absent in any combination. Also illustrated are three substantially continuous channels (5416, 5418, and 5420). A separation fluid may flow through substantially continuous channels 5416 and 5420.

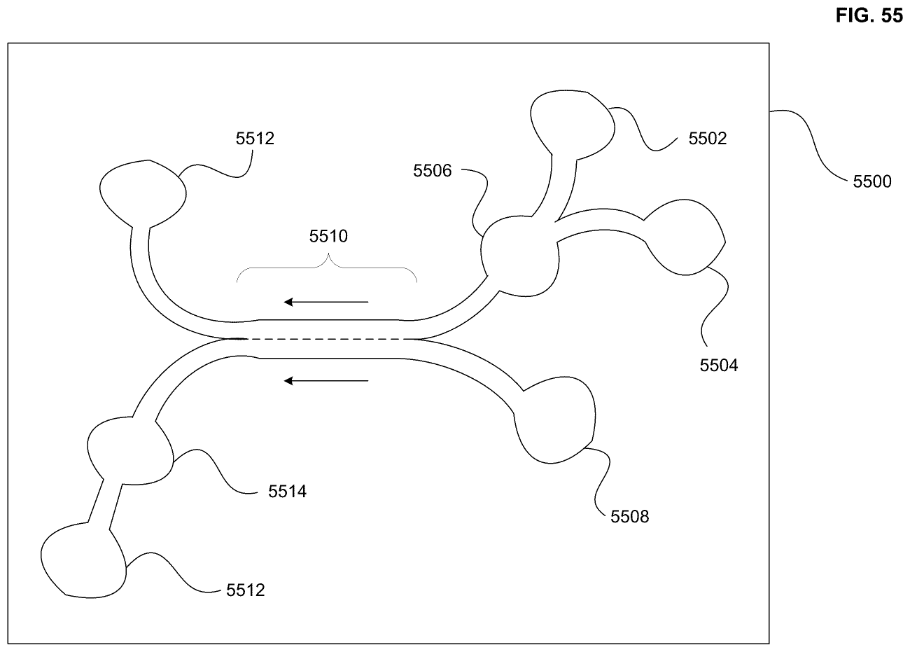

FIG. 55 illustrates an embodiment of a fluidic device placed within a microfluidic chip 5500. A sample chamber 5502 and a reagent chamber 5504 are each flowably associated with a mixing chamber 5506 that is flowably associated with a separation channel 5510 and a waste reservoir 5512. Such a configuration facilitates flow of a sample fluid 104 from the sample chamber 5502 through the separation channel 5510. A magnetically active fluid reservoir 5508 is flowably associated with the separation channel 5510, a detection chamber 5514, and a waste reservoir 5512. Such a configuration facilitates flow of magnetically active fluid 126 from the magnetically active fluid reservoir 5508 through the separation channel 5510. Flow of the sample fluid 104 and the magnetically active fluid 126 through the separation channel 5510 is indicated by the arrows as being substantially parallel.

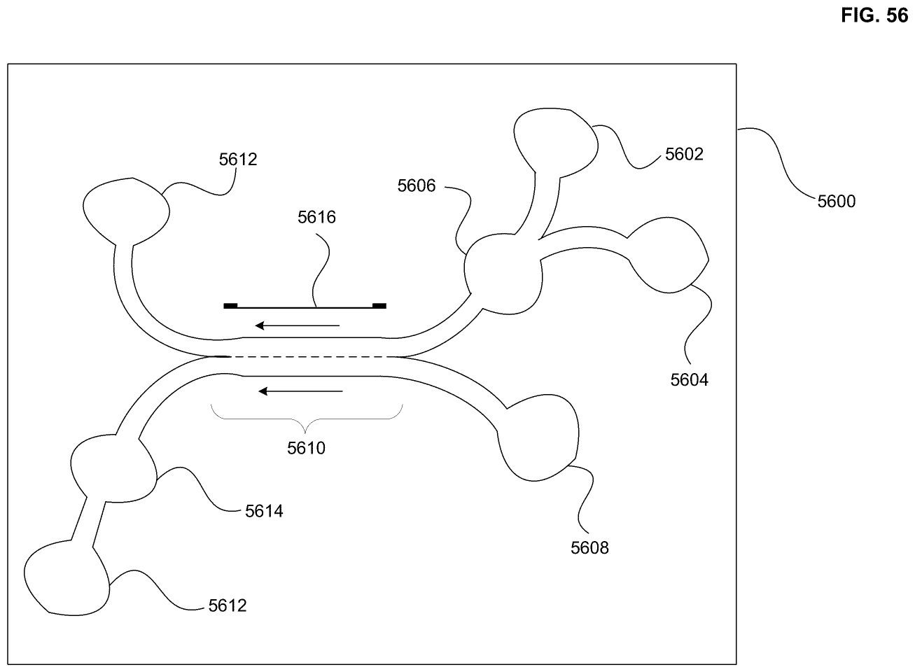

FIG. 56 illustrates an embodiment of a fluidic device placed within a microfluidic chip 5600. A sample chamber 5602 and a reagent chamber 5604 are each flowably associated with a mixing chamber 5606 that is flowably associated with a separation channel 5610 and a waste reservoir 5612. Such a configuration facilitates flow of a sample fluid 104 from the sample chamber 5602 through the separation channel 5610. A separation fluid reservoir 5608 is flowably associated with the separation channel 5610, a detection chamber 5614, and a waste reservoir 5612. Such a configuration facilitates flow of separation fluid from the fluid reservoir 5608 through the separation channel 5610. Flow of the sample fluid 104 and the separation fluid through the separation channel 5610 is indicated by the arrows as being substantially parallel. Microfluidic chip 5600 includes a magnet 5616. In some embodiments, the magnet 5616 may include an electromagnet. In some embodiments, the magnet 5616 may include a ferromagnet. In some embodiments, translocation of one or more magnetically active constituents 106 from the sample fluid 104 into the separation fluid may be facilitated by the magnet 5616. In some embodiments, such translocation may be facilitated through one or more eddy currents. In some embodiments, such translocation may be facilitated through magnetic repulsion. Accordingly, in some embodiments, such a microfluidic chip 5600 may facilitate translocation of one or more magnetically active constituents 106 from one or more samples 102 to one or more detection chambers 5614.

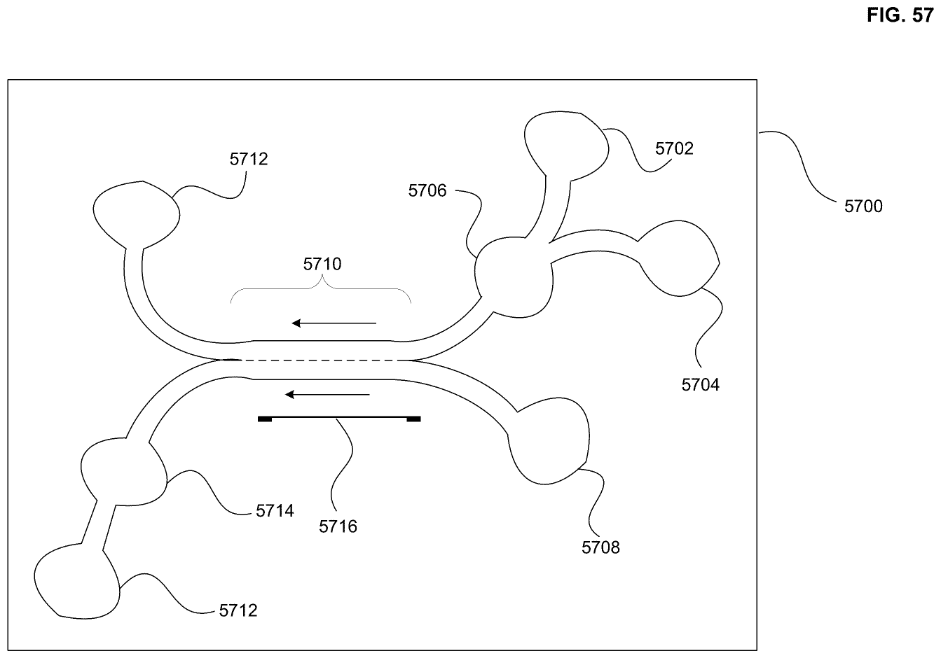

FIG. 57 illustrates an embodiment of a fluidic device placed within a microfluidic chip 5700. A sample chamber 5702 and a reagent chamber 5704 are each flowably associated with a mixing chamber 5706 that is flowably associated with a separation channel 5710 and a waste reservoir 5712. Such a configuration facilitates flow of a sample fluid 104 from the sample chamber 5702 through the separation channel 5710. A separation fluid reservoir 5708 is flowably associated with the separation channel 5710, a detection chamber 5714, and a waste reservoir 5712. Such a configuration facilitates flow of separation fluid from the fluid reservoir 5708 through the separation channel 5710. Flow of the sample fluid 104 and the separation fluid through the separation channel 5710 is indicated by the arrows as being substantially parallel. Microfluidic chip 5700 includes a magnet 5716. In some embodiments, the magnet 5716 may include an electromagnet. In some embodiments, the magnet 5716 may include a ferromagnet. In some embodiments, translocation of one or more magnetically active constituents 106 from the sample fluid 104 into the separation fluid may be facilitated by the magnet 5716. In some embodiments, such translocation may be facilitated through magnetic attraction. Accordingly, in some embodiments, such a microfluidic chip 5700 may facilitate translocation of one or more magnetically active constituents 106 from one or more samples 102 to one or more detection chambers 5714.

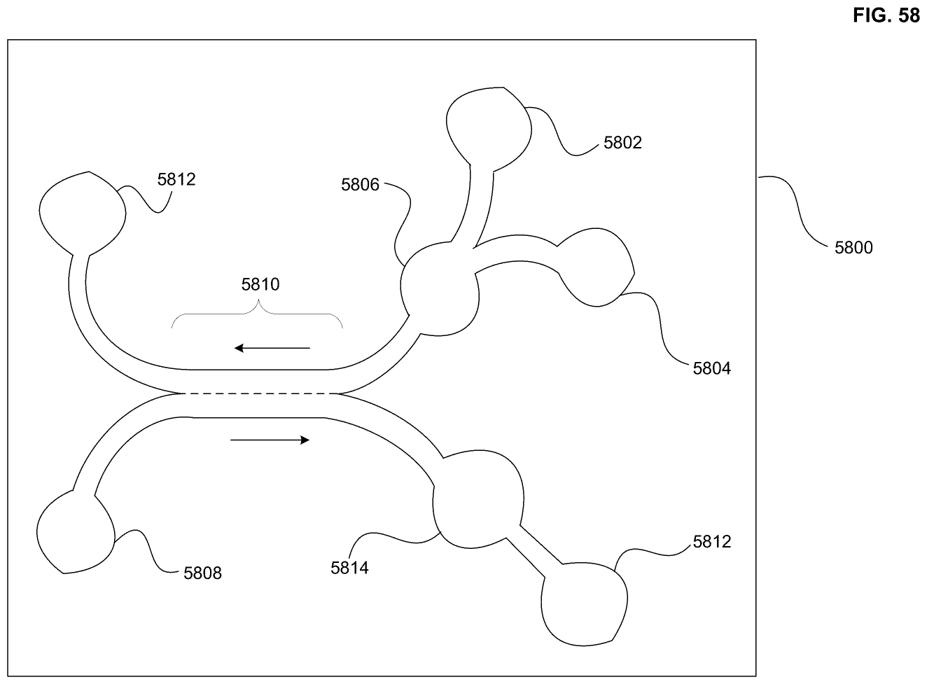

FIG. 58 illustrates an embodiment of a fluidic device placed within a microfluidic chip 5800. A sample chamber 5802 and a reagent chamber 5804 are each flowably associated with a mixing chamber 5806 that is flowably associated with a separation channel 5810 and a waste reservoir 5812. Such a configuration facilitates flow of a sample fluid 104 from the sample chamber 5802 through the separation channel 5810. A magnetically active fluid reservoir 5808 is flowably associated with the separation channel 5810, a detection chamber 5814, and a waste reservoir 5812. Such a configuration facilitates flow of magnetically active fluid 126 from the magnetically active fluid reservoir 5808 through the separation channel 5810. Flow of the sample fluid 104 and the magnetically active fluid 126 through the separation channel 5810 is indicated by the arrows as being substantially anti-parallel.

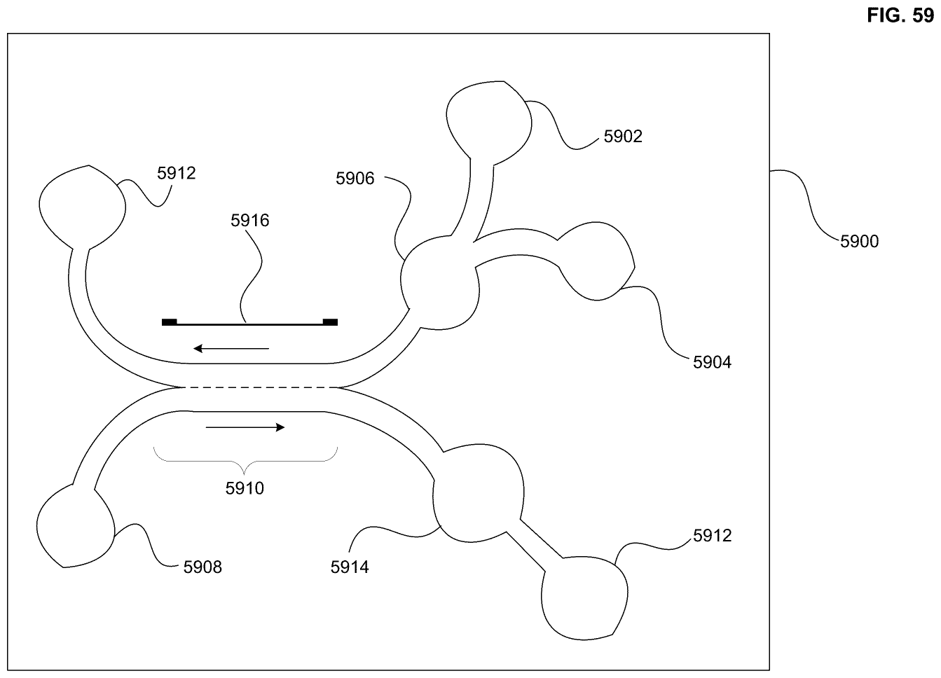

FIG. 59 illustrates an embodiment of a fluidic device placed within a microfluidic chip 5900. A sample chamber 5902 and a reagent chamber 5904 are each flowably associated with a mixing chamber 5906 that is flowably associated with a separation channel 5910 and a waste reservoir 5912. Such a configuration facilitates flow of a sample fluid 104 from the sample chamber 5902 through the separation channel 5910. A separation fluid reservoir 5908 is flowably associated with the separation channel 5910, a detection chamber 5914, and a waste reservoir 5912. Such a configuration facilitates flow of separation fluid from the fluid reservoir 5908 through the separation channel 5910. Flow of the sample fluid 104 and the separation fluid through the separation channel 5910 is indicated by the arrows as being substantially anti-parallel. Microfluidic chip 5900 includes a magnet 5916. In some embodiments, the magnet 5916 may include an electromagnet. In some embodiments, the magnet 5916 may include a ferromagnet. In some embodiments, translocation of one or more magnetically active constituents 106 from the sample fluid 104 into the separation fluid may be facilitated by the magnet 5916. In some embodiments, such translocation may be facilitated through one or more eddy currents. In some embodiments, such translocation may be facilitated through magnetic repulsion. Accordingly, in some embodiments, such a microfluidic chip 5900 may facilitate translocation of one or more magnetically active constituents 106 from one or more samples 102 to one or more detection chambers 5914.

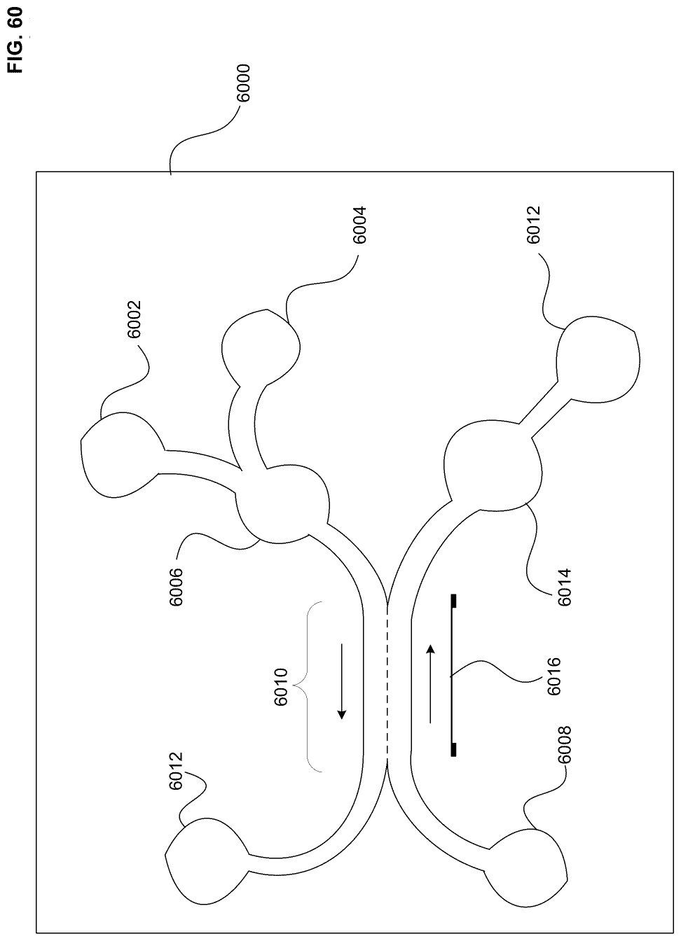

FIG. 60 illustrates an embodiment of a fluidic device placed within a microfluidic chip 6000. A sample chamber 6002 and a reagent chamber 6004 are each flowably associated with a mixing chamber 6006 that is flowably associated with a separation channel 6010 and a waste reservoir 6012. Such a configuration facilitates flow of a sample fluid 104 from the sample chamber 6002 through the separation channel 6010. A separation fluid reservoir 6008 is flowably associated with the separation channel 6010, a detection chamber 6014, and a waste reservoir 6012. Such a configuration facilitates flow of separation fluid from the fluid reservoir 6008 through the separation channel 6010. Flow of the sample fluid 104 and the separation fluid through the separation channel 6010 is indicated by the arrows as being substantially anti-parallel. Microfluidic chip 6000 includes a magnet 6016. In some embodiments, the magnet 6016 may include an electromagnet. In some embodiments, the magnet 6016 may include a ferromagnet. In some embodiments, translocation of one or more magnetically active constituents 106 from the sample fluid 104 into the separation fluid may be facilitated by the magnet 6016. In some embodiments, such translocation may be facilitated through magnetic attraction. Accordingly, in some embodiments, such a microfluidic chip 6000 may facilitate translocation of one or more magnetically active constituents 106 from one or more samples 102 to one or more detection chambers 6014.

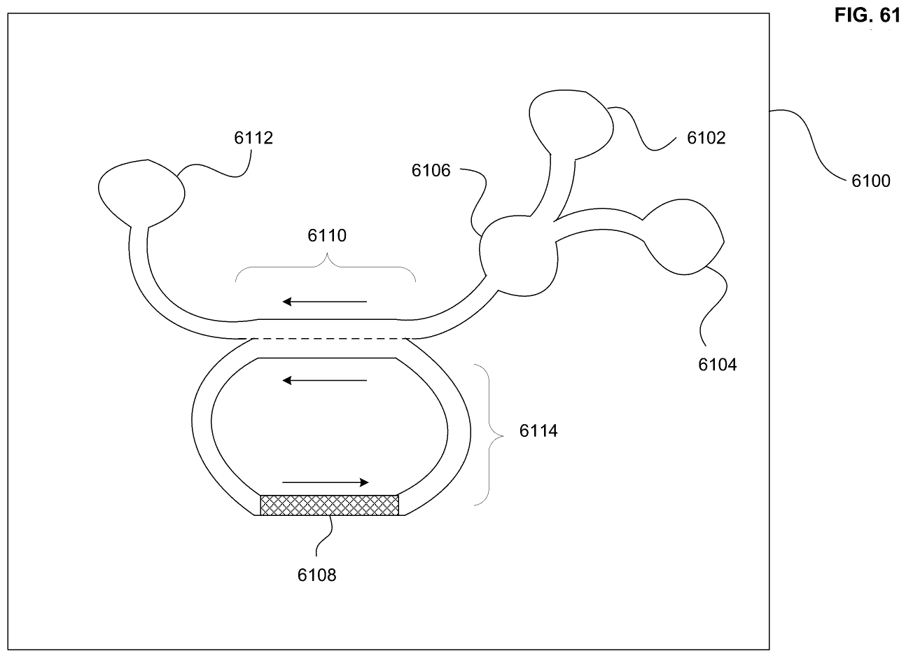

FIG. 61 illustrates an embodiment of a fluidic device placed within a microfluidic chip 6100. A sample chamber 6102 and a reagent chamber 6104 are each flowably associated with a mixing chamber 6106 that is flowably associated with a separation channel 6110 and a waste reservoir 6112. Such a configuration facilitates flow of a sample fluid 104 from the sample chamber 6102 through the separation channel 6110. A continuous channel 6114 is flowably associated with the separation channel 6110 and a detection chamber 6108. Such a configuration provides for continuous flow of magnetically active fluid 126 through the separation channel 6110. Flow of the sample fluid 104 and the magnetically active fluid 126 through the separation channel 6110 is indicated by the arrows as being substantially parallel.

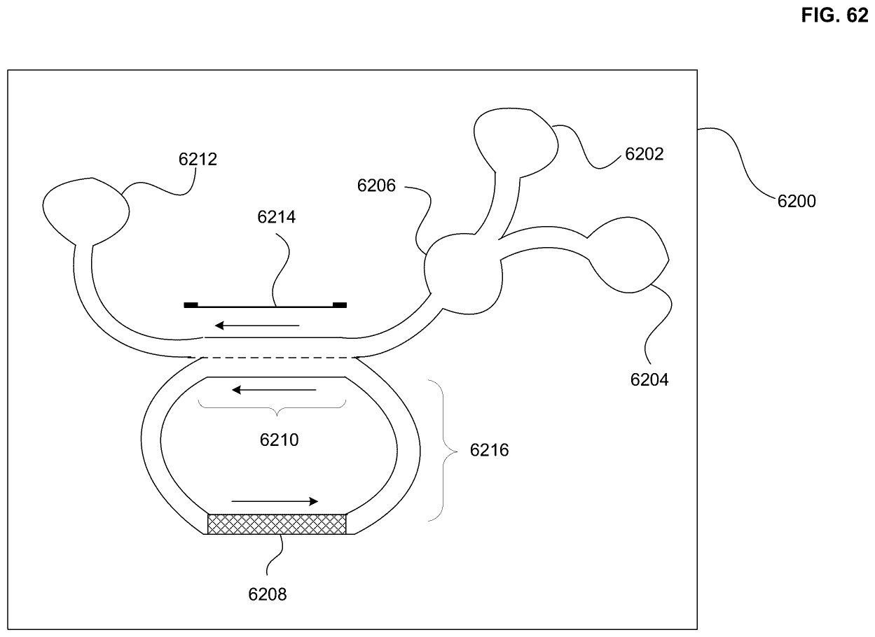

FIG. 62 illustrates an embodiment of a fluidic device placed within a microfluidic chip 6200. A sample chamber 6202 and a reagent chamber 6204 are each flowably associated with a mixing chamber 6206 that is flowably associated with a separation channel 6210 and a waste reservoir 6212. Such a configuration facilitates flow of a sample fluid 104 from the sample chamber 6202 through the separation channel 6210. A continuous channel 6216 is flowably associated with the separation channel 6210 and a detection chamber 6208. Such a configuration provides for continuous flow of separation fluid through the separation channel 6210. Flow of the sample fluid 104 and the separation fluid through the separation channel 6210 is indicated by the arrows as being substantially parallel. Microfluidic chip 6200 includes a magnet 6214. In some embodiments, the magnet 6214 may include an electromagnet. In some embodiments, the magnet 6214 may include a ferromagnet. In some embodiments, translocation of one or more magnetically active constituents 106 from the sample fluid 104 into the separation fluid may be facilitated by the magnet 6214. In some embodiments, such translocation may be facilitated through one or more eddy currents. In some embodiments, such translocation may be facilitated through magnetic repulsion. Accordingly, in some embodiments, such a microfluidic chip 6200 may facilitate translocation of one or more magnetically active constituents 106 from one or more samples 102 to one or more detection chambers 6208.

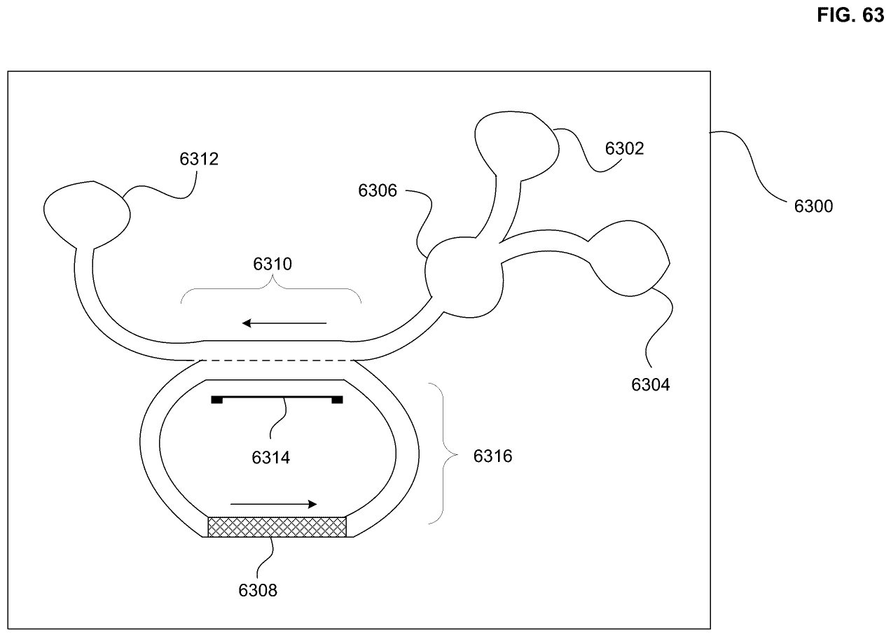

FIG. 63 illustrates an embodiment of a fluidic device placed within a microfluidic chip 6300. A sample chamber 6302 and a reagent chamber 6304 are each flowably associated with a mixing chamber 6306 that is flowably associated with a separation channel 6310 and a waste reservoir 6312. Such a configuration facilitates flow of a sample fluid 104 from the sample chamber 6302 through the separation channel 6310. A continuous channel 6316 is flowably associated with the separation channel 6310 and a detection chamber 6308. Such a configuration provides for continuous flow of separation fluid through the separation channel 6310. Flow of the sample fluid 104 and the separation fluid through the separation channel 6310 is indicated by the arrows as being substantially parallel. Microfluidic chip 6300 includes a magnet 6314. In some embodiments, the magnet 6314 may include an electromagnet. In some embodiments, the magnet 6314 may include a ferromagnet. In some embodiments, translocation of one or more magnetically active constituents 106 from the sample fluid 104 into the separation fluid may be facilitated by the magnet 6314. In some embodiments, such translocation may be facilitated through magnetic attraction. Accordingly, in some embodiments, such a microfluidic chip 6300 may facilitate translocation of one or more magnetically active constituents 106 from one or more samples 102 to one or more detection chambers 6308.

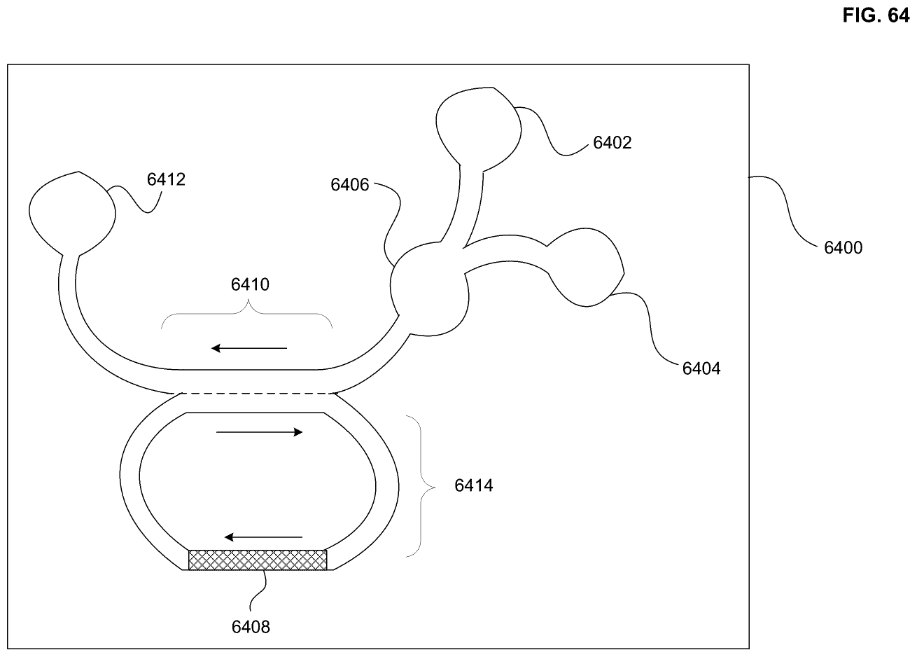

FIG. 64 illustrates an embodiment of a fluidic device placed within a microfluidic chip 6400. A sample chamber 6402 and a reagent chamber 6404 are each flowably associated with a mixing chamber 6406 that is flowably associated with a separation channel 6410 and a waste reservoir 6412. Such a configuration facilitates flow of a sample fluid 104 from the sample chamber 6402 through the separation channel 6410. A continuous channel 6414 is flowably associated with the separation channel 6410 and a detection chamber 6408. Such a configuration provides for continuous flow of magnetically active fluid 126 through the separation channel 6410. Flow of the sample fluid 104 and the magnetically active fluid 126 through the separation channel 6410 is indicated by the arrows as being substantially anti-parallel.

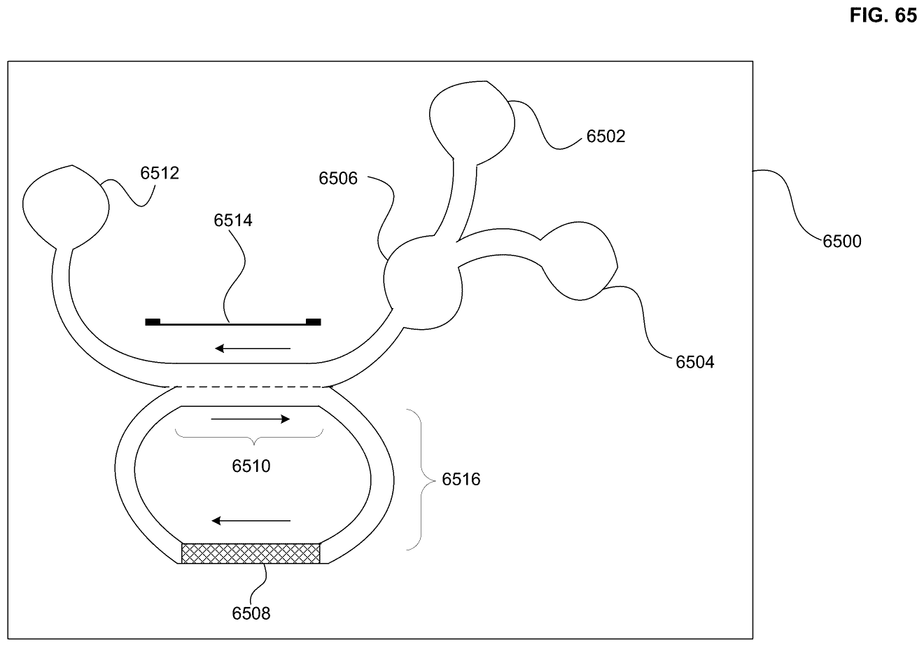

FIG. 65 illustrates an embodiment of a fluidic device placed within a microfluidic chip 6500. A sample chamber 6502 and a reagent chamber 6504 are each flowably associated with a mixing chamber 6506 that is flowably associated with a separation channel 6510 and a waste reservoir 6512. Such a configuration facilitates flow of a sample fluid 104 from the sample chamber 6502 through the separation channel 6510. A continuous channel 6516 is flowably associated with the separation channel 6510 and a detection chamber 6508. Such a configuration provides for continuous flow of separation fluid through the separation channel 6510. Flow of the sample fluid 104 and the separation fluid through the separation channel 6510 is indicated by the arrows as being substantially anti-parallel. Microfluidic chip 6500 includes a magnet 6514. In some embodiments, the magnet 6514 may include an electromagnet. In some embodiments, the magnet 6514 may include a ferromagnet. In some embodiments, translocation of one or more magnetically active constituents 106 from the sample fluid 104 into the separation fluid may be facilitated by the magnet 6514. In some embodiments, such translocation may be facilitated through one or more eddy currents. In some embodiments, such translocation may be facilitated through magnetic repulsion. Accordingly, in some embodiments, such a microfluidic chip 6500 may facilitate translocation of one or more magnetically active constituents 106 from one or more samples 102 to one or more detection chambers 6508.

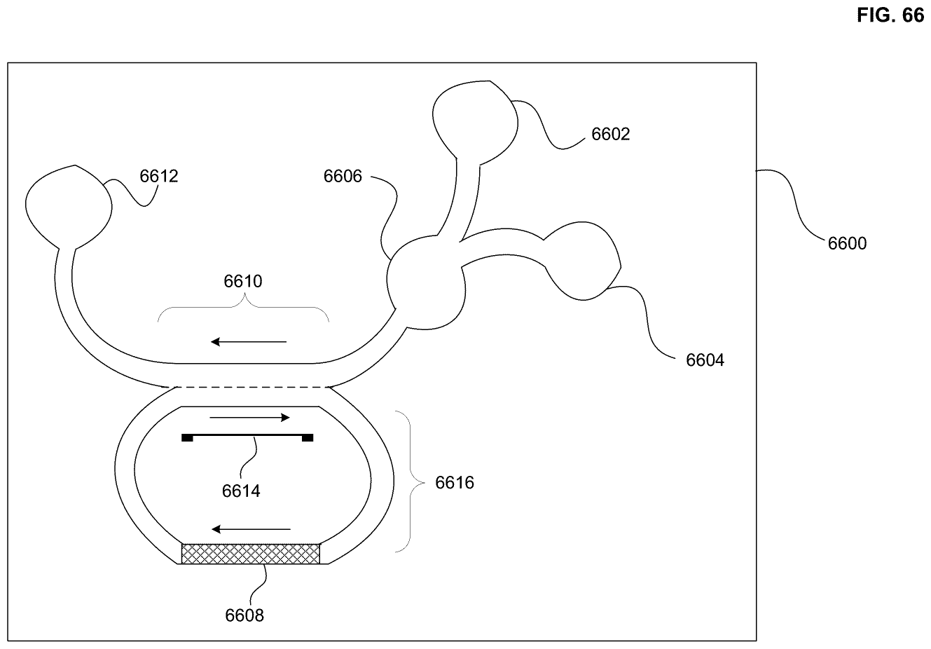

FIG. 66 illustrates an embodiment of a fluidic device placed within a microfluidic chip 6600. A sample chamber 6602 and a reagent chamber 6604 are each flowably associated with a mixing chamber 6606 that is flowably associated with a separation channel 6610 and a waste reservoir 6612. Such a configuration facilitates flow of a sample fluid 104 from the sample chamber 6602 through the separation channel 6610. A continuous channel 6616 is flowably associated with the separation channel 6610 and a detection chamber 6608. Such a configuration provides for continuous flow of separation fluid through the separation channel 6610. Flow of the sample fluid 104 and the separation fluid through the separation channel 6610 is indicated by the arrows as being substantially anti-parallel. Microfluidic chip 6600 includes a magnet 6614. In some embodiments, the magnet 6614 may include an electromagnet. In some embodiments, the magnet 6614 may include a ferromagnet. In some embodiments, translocation of one or more magnetically active constituents 106 from the sample fluid 104 into the separation fluid may be facilitated by the magnet 6614. In some embodiments, such translocation may be facilitated through magnetic attraction. Accordingly, in some embodiments, such a microfluidic chip 6600 may facilitate translocation of one or more magnetically active constituents 106 from one or more samples 102 to one or more detection chambers 6608.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electro-mechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include, but are not limited to, a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a voice-over IP system, etc.), (f) a business entity (e.g., an Internet Service Provider (ISP) entity such as Comcast Cable, Quest, Southwestern Bell, etc), or (g) a wired/wireless services entity such as Sprint, Cingular, Nextel, etc.), etc.

Although a user 138 is shown/described herein as a single illustrated figure, those skilled in the art will appreciate that a user 138 may be representative of a human user 138, a robotic user 138 (e.g., computational entity), and/or substantially any combination thereof (e.g., a user 138 may be assisted by one or more robotic agents). In addition, a user 138 as set forth herein, although shown as a single entity may in fact be composed of two or more entities. Those skilled in the art will appreciate that, in general, the same may be said of “sender” and/or other entity-oriented terms as such terms are used herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to, physically mateable and/or physically interacting components and/or wirelessly interactable and/or wirelessly interacting components and/or logically interacting and/or logically interactable components.

All of the above U.S. patents, U.S. patent application publications, U.S. patent applications, foreign patents, foreign patent applications and non-patent publications referred to in this specification, are incorporated herein by reference, in their entireties.

What is claimed is:

1. A method comprising:
placing one or more sample fluids into one or more separation channels so that the one or more sample fluids are in substantially laminar flow with one or more magnetically active fluids; and
translocating one or more magnetically active constituents from the one or more sample fluids into the one or more magnetically active fluids.
